(19) United States
(12) Patent Application Publication
(10) Pub. No.: US 2026/0354852 A1
(43) Pub. Date: Nov. 12, 2026

(54) PERIPHERAL INTRAVENOUS LITHOTRIPSY SYSTEM CAPABLE OF USE OVER 0.025 TO 0.095-INCH GUIDEWIRE

(71) Applicant: NEXTERN INNOVATION, LLC, White Bear Lake, MN (US)

(72) Inventors: Alexander Thome, Minnetonka, MN (US); Michael Brenzel, Maple Grove, MN (US)

(73) Assignee: NEXTERN INNOVATION, LLC, White Bear Lake, MN (US)

(21) Appl. No.: 19/669,569

(22) Filed: May 6, 2026

Related U.S. Application Data
(60) Provisional application No. 63/802,355, filed on May 8, 2025.
(60) Provisional application No. 64/014,694, filed on Mar. 23, 2026.

(51) Int. Cl. A61B 17/22; A61B 17/00

(52) U.S. Cl. CPC A61B 17/22022; A61B 17/00234; A61B 2017/00238; A61B 2017/00305; A61B 2017/00929; A61B 2017/22025; A61B 2017/22038; A61B 2017/22065

(57) ABSTRACT

Apparatus and related methods relate to an intravascular lithotripsy (IVL) catheter configured for use with a peripheral-class guidewire. In an illustrative embodiment, an IVL catheter may include a guidewire lumen sized to receive a guidewire having a diameter of at least 0.035 inches (0.889 mm). The catheter may, for example, include one or more emitter modules disposed along an inner shaft within an inflatable balloon. The emitter modules may, for example, include an emitter body and a discharge conductor arranged to generate electrical sparks within the balloon. The inner shaft may, for example, include conductor routing arranged outside the guidewire lumen, such as along peripheral wall regions of the inner shaft. Various embodiments may advantageously allow the IVL catheter to be used over a peripheral-class guidewire without a corresponding increase in crossing profile.

### CROSS-REFERENCE TO RELATED APPLICATIONS

This application claims the benefit of and incorporates by reference the entirety both of U.S. Provisional Application No. 64/014,694, filed Mar. 23, 2026, and of U.S. Provisional Application No. 63/802,355, filed May 8, 2025.

Unless expressly stated, changes in terminology from the provisional applications to this application are made without prejudice or disclaimer of subject matter. Changes from the provisional applications are intended to be broadening and/or additive unless expressly stated otherwise. Replacements of alternative terms with a single representative term, for example, are inclusive unless otherwise defined. Embodiments of similar languages in this application are not modifications or disclaimer of the embodiments disclosed in previous incorporated disclosures unless otherwise stated.

### BACKGROUND

Intravascular lithotripsy (IVL) may be used to treat calcified lesions within blood vessels, including peripheral vessels such as the superficial femoral artery, common femoral artery, and iliac arteries, as well as coronary vessels. Calcified vascular lesions may present challenges for interventional procedures, as calcium deposits within vessel walls may resist conventional balloon dilation and may complicate delivery and deployment of endovascular devices such as stents and stent grafts. IVL systems may address such calcification by generating acoustic pressure waves within an inflatable balloon positioned at a target treatment site, where the pressure waves may fracture calcium deposits within the vessel wall, such as to facilitate subsequent vessel preparation and device delivery.

IVL catheters may be used across a range of clinical scenarios, including treatment of heavily calcified peripheral arterial disease in the lower extremities, preparation of calcified landing zones prior to endovascular aortic repair, and treatment of calcified coronary lesions prior to stenting

IVL systems may include a catheter body, an inflatable balloon, and one or more emitter modules positioned along the catheter within the balloon. Emitter modules may include electrode assemblies configured to generate electrical sparks within a fluid-filled balloon, where rapid vaporization of fluid at the spark location may generate acoustic pressure waves that propagate radially outward through the balloon and vessel wall. Electrode assemblies may include two or more conductors, such as a conductor (e.g., wire electrode) and an emitter body (e.g., a ring electrode), or other conductor configurations, separated by a spark gap across which electrical discharge may occur when a sufficient voltage is applied. Some IVL systems may include multiple emitter modules, such as arranged along the catheter. Pulse generators connected to the catheter may supply high-voltage electrical pulses to the emitter modules.

### TECHNICAL FIELD

Apparatus and methods generally relate to intravascular lithotripsy, including, by way of example and not limitation, lithotripsy in peripheral vasculature.

### BRIEF SUMMARY

Apparatus and related methods relate to an intravascular lithotripsy system that includes a catheter extending along a longitudinal axis, defining a guidewire lumen configured to receive a 0.025-inch (0.635 mm) to 0.095-inch (2.413 mm) guidewire. An inner shaft is disposed within the catheter. At least four emitter modules are disposed along the inner shaft. Each emitter module includes an emitter body having an emitter body surface extending along the longitudinal axis and at least one conductor having a conductor surface spaced from the emitter body surface by a spark gap. A dielectric material insulates the at least one conductor from the emitter body surface except at a discharge location.

Apparatus and related methods relate to an intravascular lithotripsy (IVL) balloon catheter. In an illustrative embodiment, the IVL balloon catheter includes a catheter shaft extending between a proximal end and a distal end and defining a guidewire lumen sized to receive a 0.025-inch (0.635 mm) to 0.095-inch (2.413 mm) guidewire. An inflatable balloon is mounted to the catheter shaft and defines a balloon interior volume. At least five lithotripsy emitter bodies are disposed within the balloon interior volume. Each emitter body includes an electrically conductive body mounted about an inner shaft of the catheter. At least one conductor channel is configured to route a conductor longitudinally through the emitter body, and an exposed metal region exposing an emitter body surface and a conductor surface through a dielectric encapsulation.

Apparatus and related methods relate to an intravascular lithotripsy (IVL) catheter configured for use with a peripheral-class guidewire. In an illustrative embodiment, an IVL catheter may include a guidewire lumen sized to receive a guidewire having a diameter of at least 0.035 inches (0.889 mm). The catheter may, for example, include one or more emitter modules disposed along an inner shaft within an inflatable balloon. The emitter modules may, for example, include an emitter body and a discharge conductor arranged to generate electrical sparks within the balloon. The inner shaft may, for example, include conductor routing arranged outside the guidewire lumen, such as along peripheral wall regions of the inner shaft. Various embodiments may advantageously allow the IVL catheter to be used over a peripheral-class guidewire without a corresponding increase in crossing profile.

Apparatus and related methods relate to an intravascular lithotripsy (IVL) emitter in which opposing spark gap poles are positioned co-radially relative to a catheter centerline. In an illustrative embodiment, an emitter module may include an emitter body having a discharge aperture including a first exposed discharge surface. The emitter module may, for example, include a discharge conductor having a second exposed discharge surface within the discharge aperture. The first and second exposed discharge surfaces may, for example, be positioned such that a single arcuate surface concentric with a longitudinal centerline of the catheter passes through both exposed discharge surfaces. Various embodiments may advantageously reduce crossing profile of the catheter, increase available lumen size, and/or increase control of spark initiation location.

Apparatus and related methods relate to an intravascular lithotripsy (IVL) emitter in which an encapsulating dielectric material physically separates electrode conductors except at defined exposed discharge surfaces. In an illustrative embodiment, an emitter module may include an emitter body having a conductor channel extending longitudinally and a discharge aperture opening to the conductor channel. The discharge aperture may, for example, include a first exposed discharge surface. A discharge conductor may, for example, extend through the conductor channel and include a second exposed discharge surface within the discharge aperture. The encapsulating dielectric material may, for example, separate the emitter body from the discharge conductor such that electrical discharge is physically inhibited except between the first and second exposed discharge surfaces. Various embodiments may advantageously cause the location of electrical discharge to be determined by structure rather than by random breakdown, such as increasing predictability of spark initiation across successive discharge events.

Apparatus and related methods relate to an intravascular lithotripsy (IVL) emitter having a constrained ratio of exposed discharge surface areas between opposing electrode members. In an illustrative embodiment, an emitter module may include an emitter body having a discharge aperture including a first exposed discharge surface. The emitter module may, for example, include a discharge conductor having a second exposed discharge surface within the discharge aperture. The first and second exposed discharge surfaces may, for example, be configured such that a ratio of the surface area of the first exposed discharge surface to the surface area of the second exposed discharge surface is less than or equal to 10:1. Various embodiments may advantageously promote more symmetric charge distribution between opposing electrodes, such as reducing variability in spark initiation location and/or increasing repeatability of pressure wave output.

Apparatus and related methods relate to an intravascular lithotripsy (IVL) emitter in which electrical discharge initiation voltage and post-breakdown discharge current magnitude are independently controlled by electrode geometry. In an illustrative embodiment, an emitter module may include a first electrode member and a second electrode member having opposing exposed discharge surfaces defining an electrical discharge region. The exposed discharge surfaces may, for example, define a first edge-to-edge distance less than a second edge-to-edge distance at a laterally offset location, the first edge-to-edge distance determining a breakdown voltage. The first electrode member may, for example, include a channel width defined by opposing channel walls bounding the discharge aperture, such that breakdown voltage and discharge current magnitude are each determined independently by a respective one of those geometric parameters. Various embodiments may advantageously enable more precise control of therapeutic pressure wave characteristics, such as with standard and/or invariable pulse generators.

Apparatus and related methods relate to an intravascular lithotripsy (IVL) emitter module in which the discharge conductor diameter is equal or approximately equal to the wall thickness of the emitter body, with an open channel conductor retention geometry. In an illustrative embodiment, an emitter module may include an emitter body with an interrupted annular cross sectional shape. The emitter module may, for example, have an elongated toroidal or frusto toroidal shape. The emitter module may, for example, have an outer surface and an inner surface. The emitter body may, for example, include a conductor channel and a discharge aperture. A discharge conductor may, for example, extend through the conductor channel with an outermost diameter at least eighty percent of the emitter body wall thickness, positioned entirely within the volumetric shell bounded by the outer and inner surfaces. A retention region of the conductor channel may, for example, be narrower at the outer surface than at mid-depth. Various embodiments may advantageously reduce radial profile, increase available guidewire lumen, and/or retain the discharge conductor without additional components.

Apparatus and related methods relate to an intravascular lithotripsy (IVL) emitter module in which a conductor channel follows a non-linear path relative to the longitudinal axis of the emitter body. In an illustrative embodiment, an emitter module may include an emitter body having a conductor channel. The conductor channel may, for example, follow a non-linear path in a circumferential direction relative to the emitter body. A discharge conductor may, for example, be disposed within the conductor channel such that the discharge conductor deviates from a straight radial or chordal alignment. Various embodiments may advantageously distribute spark initiation locations and electrode erosion along a non-linear path, such as extending emitter operational life and/or distributing pressure wave generation around more or all of the emitter circumference.

The details of various embodiments are set forth in the accompanying drawings and the description below. Other features and advantages will be apparent from the description and drawings, and from the claims.

### BRIEF DESCRIPTION OF THE DRAWINGS

Various embodiments of the present embodiments are described with reference to the following figures.

FIG. 1 depicts an example embodiment of an intravascular lithotripsy (IVL) device (such as, for example, for peripheral applications) including a progressive spark surface emitter (PSSE), an emitter body peripherally tangential conductor arrangement, and an intra-catheter conductor configuration.

FIG. 2 depicts an example PSSE module mounted on a catheter shaft, illustrating an emitter body and a conductor extending therethrough.

FIG. 3 depicts perspective views of an example PSSE module illustrating an emitter body having a window configured to expose conductive surfaces.

FIG. 4 depicts an example PSSE module pre-encapsulation.

FIG. 5 depicts axial views of example emitter body configurations illustrating internal lumen geometry and conductor positioning.

FIGS. 6A-6B depict longitudinal views of an example PSSE module illustrating axial dimensions and locations of spark-generating regions.

FIG. 7 depicts a detailed view of an example spark-generating region illustrating a conductor positioned relative to an exposed emitter body surface.

FIG. 8A and FIG. 8B depict an example PSSE module illustrating a reduced-width spark aperture geometry.

FIG. 9 depicts an example PSSE module illustrating an emitter body geometry affecting spark gap characteristics.

FIG. 10 depicts an example PSSE module illustrating a windowless emitter configuration.

FIG. 11 depicts a perspective view of an example windowless PSSE module illustrating a progressive spark surface path.

FIG. 12 depicts an axial cross-sectional view of an example PSSE module positioned within an inflated balloon on a catheter shaft.

FIG. 13 depicts an example PSSE array including multiple PSSE modules arranged along a catheter shaft.

FIG. 14 depicts an example electrical schematic illustrating series coupling of multiple PSSE modules and associated spark gaps.

FIG. 15 depicts a longitudinal view of an example catheter shaft assembly illustrating an outer shaft and a guidewire lumen.

FIG. 16 depicts an example emission cycle plot.

FIG. 17 depicts an example method of configuring a PSSE.

FIG. 18 depicts an example method of operating a PSSE.

FIG. 19A depicts an example embodiment of a catheter including a ring electrode member and a wire electrode member disposed on a catheter inner body and extending along a longitudinal centerline of the catheter.

FIG. 19B depicts a cross-section of FIG. 19A in perspective view. FIG. 19C and FIG. 19D depict the cross-section in elevation view including example geometrical relationships.

FIG. 20A and FIG. 20B depict close-up cross-section views (e.g., of the embodiment in FIG. 19A) depicting example spark initiation radius geometrical relationships.

FIG. 21 depicts an example close-up perspective view of an example PSSE embodiment (e.g., of the embodiment in FIG. 19A), illustrating a spark initiation region.

FIG. 22 depicts an example embodiment (e.g., such as the embodiment of FIG. 21) with equal exposed discharge surface areas of a wire electrode and ring electrode.

FIG. 23A depicts a top plan view, and FIG. 23B depicts a cross-section view, of the embodiment of FIG. 21, depicting example geometric relationships related to spark gaps.

FIG. 24, FIG. 25, and FIG. 26 depict a top plan view, cross-section view, and close-up cross-section view, respectively, depicting example geometric relationships related to spark gaps of an embodiment with a wider exposed area (e.g., relative to the example in FIG. 21).

FIG. 27A, FIG. 27B, and FIG. 27C depict a perspective view, cross-section view, and cross-section view with example wire conductor placement, respectively, of an example embodiment with shaped channels (e.g., conductor retentive shaping).

FIG. 28 depicts a close-up cross-section view of an example embodiment with shaped channel (e.g., curvilinear channel cross-section geometry).

FIG. 29 depicts various views of an example emitter body with a non-linear channel.

FIG. 30A and FIG. 30B depict an overall perspective view and a close-up perspective view, respectively, of an example emitter body with curvilinear channel boundaries.

FIG. 31 depicts an example method of making an emitter module.

FIG. 32 depicts an example plot of acoustic pressure output versus spark gap distance for an example IVL-class emitter circuit.

FIG. 33 depicts example voltage-time traces for multiple spark gap distances for an example IVL-class emitter circuit.

FIG. 34 depicts an example energy penalty diagram showing available energy fraction as a function of spark gap distance.

FIG. 35 depicts an example cross-section showing an adhesive and encapsulating material.

FIG. 36 depicts comparative cross-sections illustrating deformation behavior of two encapsulating material configurations during a spark generation event.

Like reference numerals refer to like parts throughout the various views unless otherwise specified. Embodiments and portions of embodiments illustrated and described herein are non-limiting and non-exhaustive.

### DETAILED DESCRIPTION

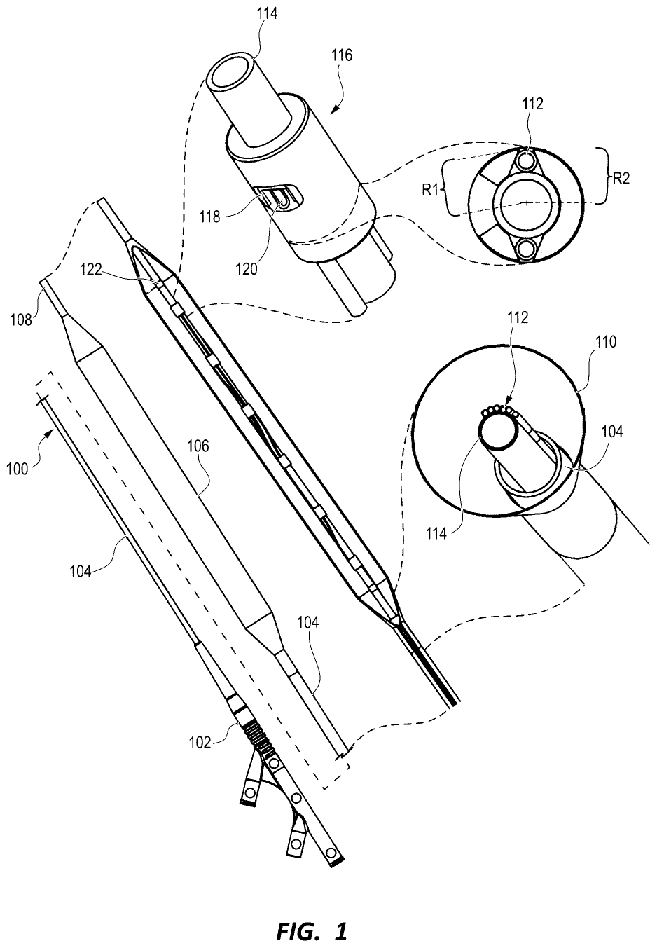

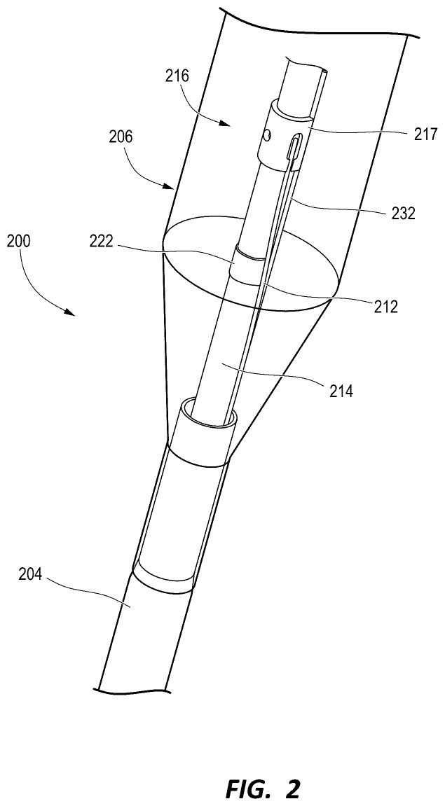

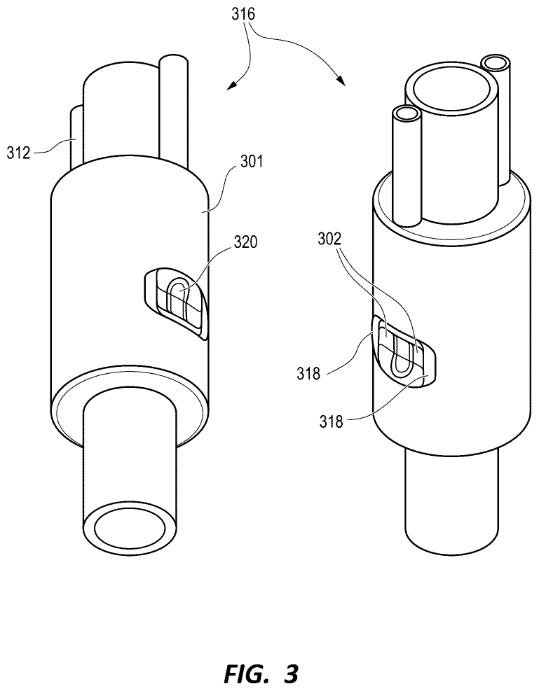

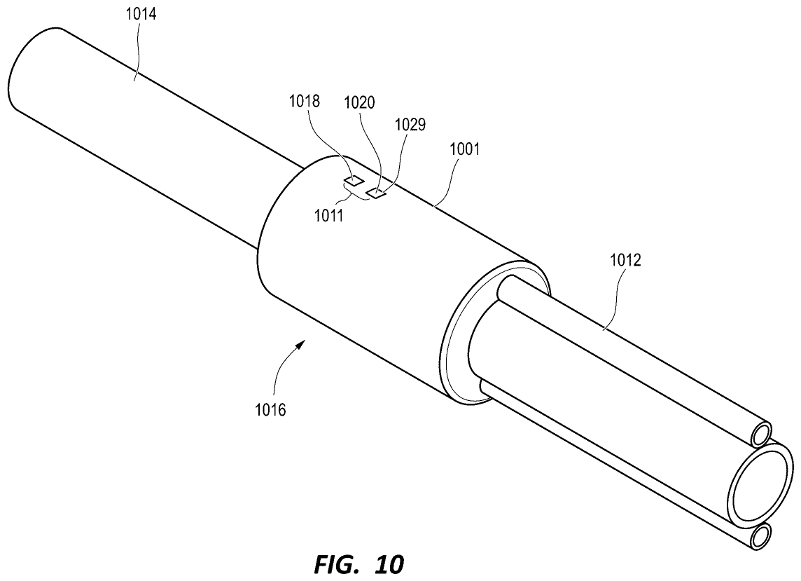

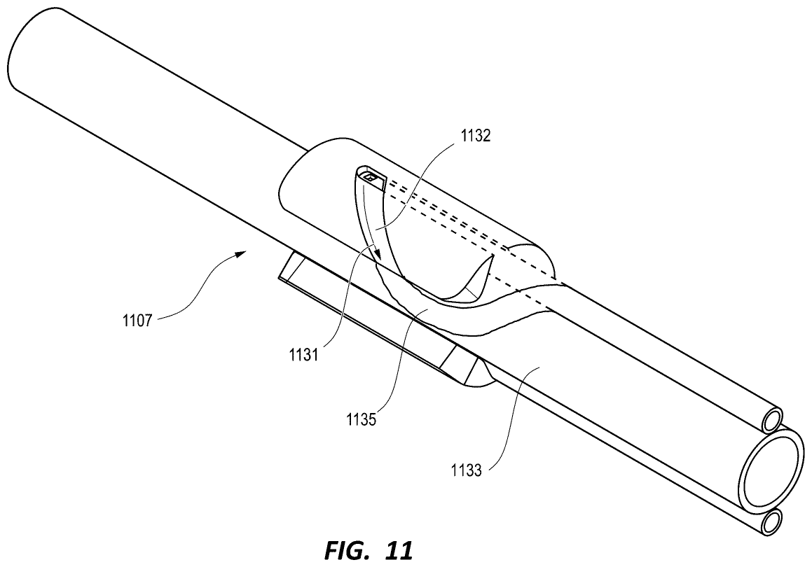

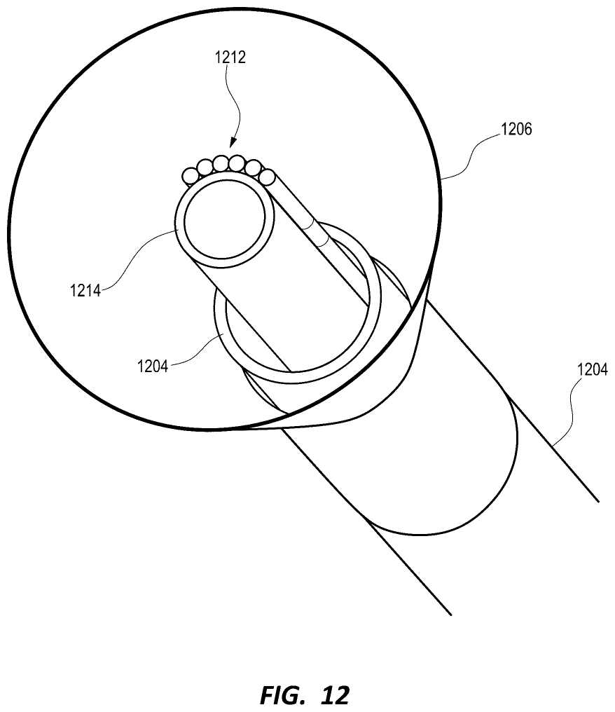

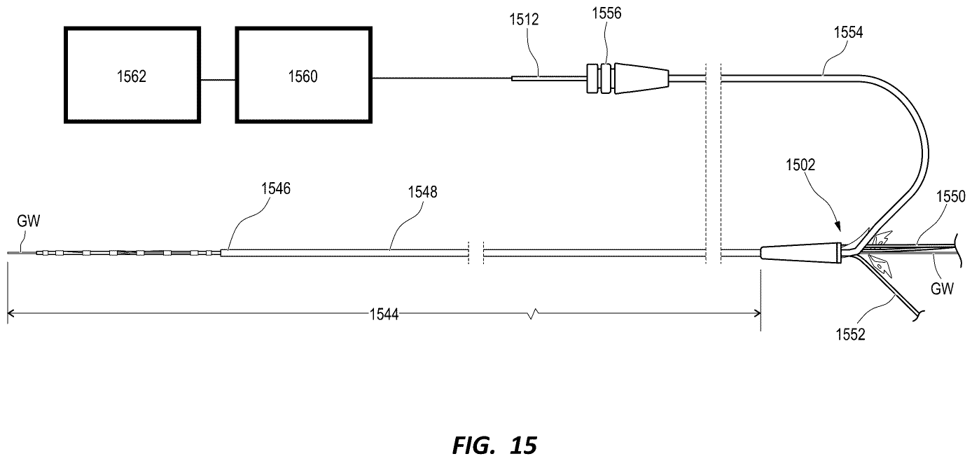

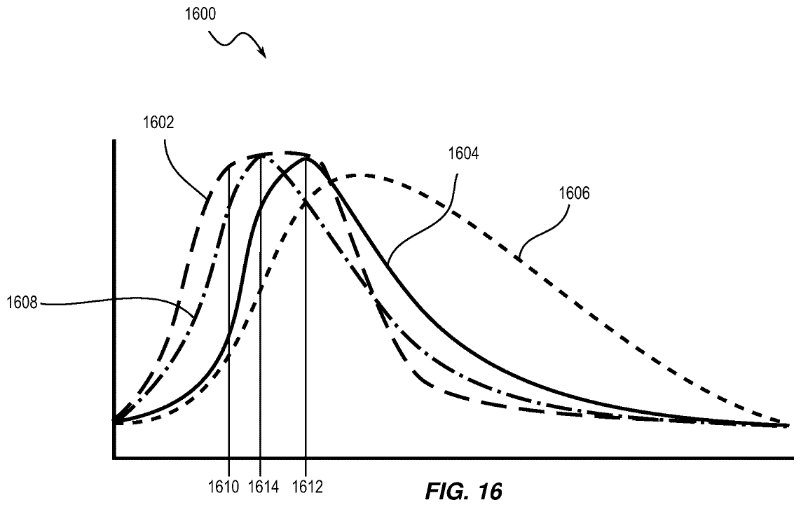

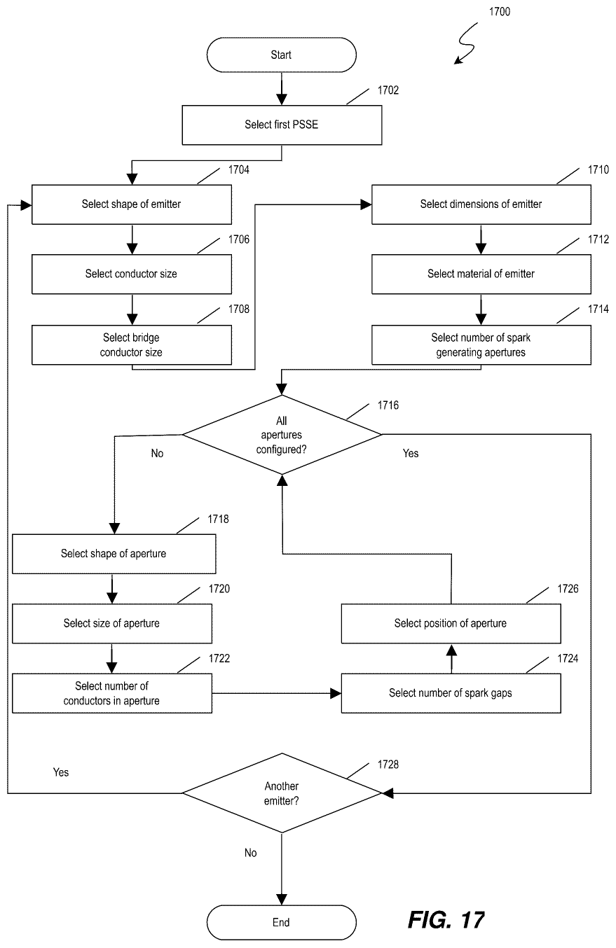

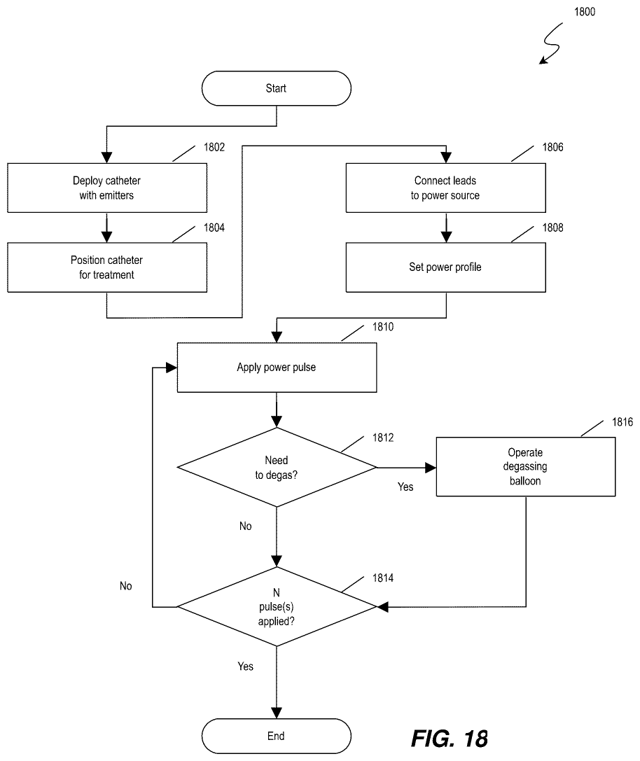

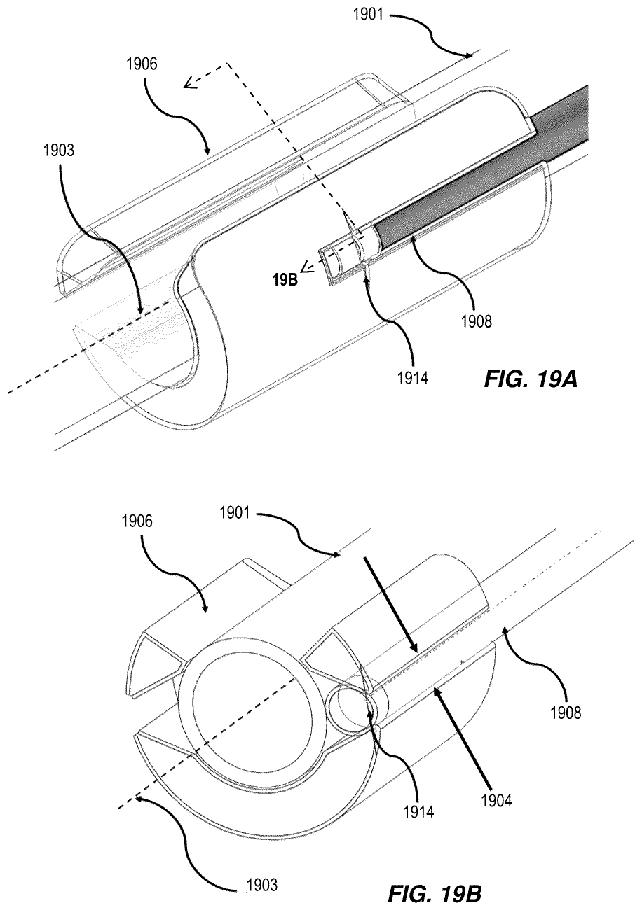

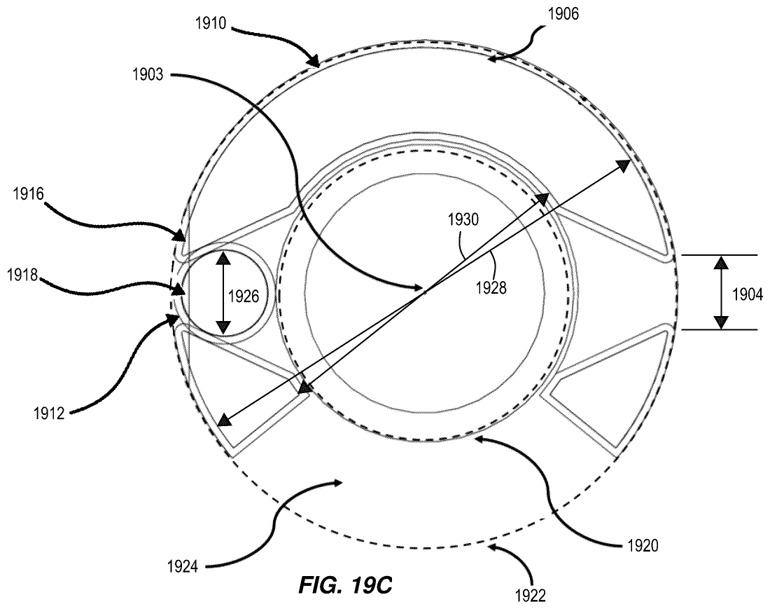

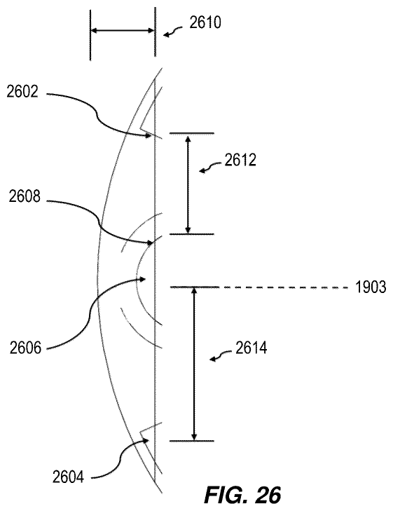

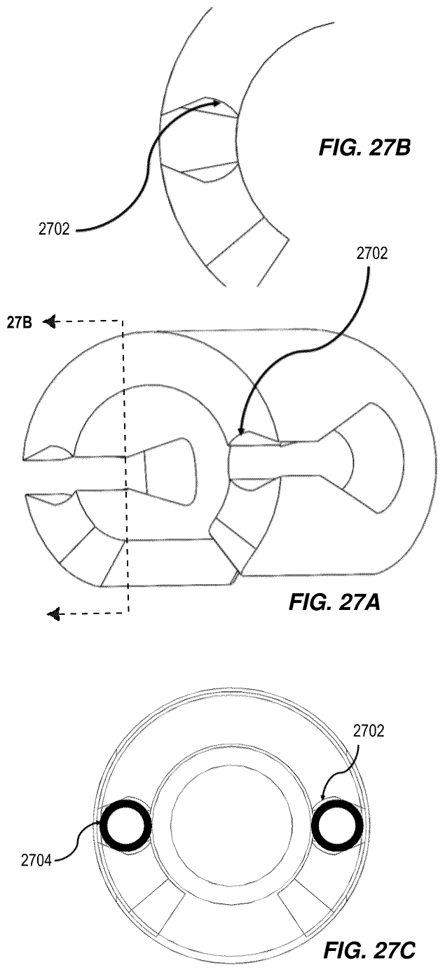

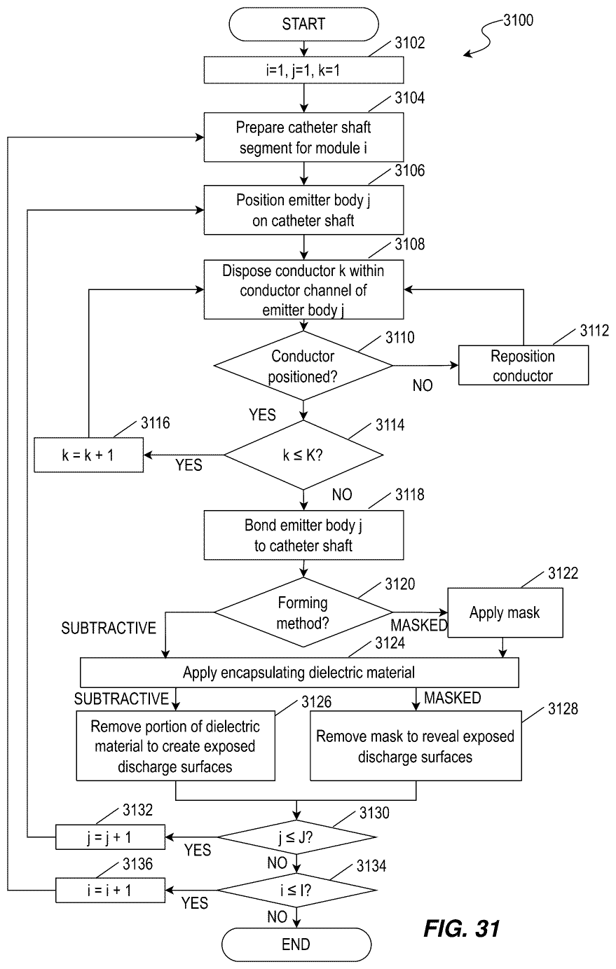

In order to assist rapid comprehension, this document introduces an example intravascular lithotripsy (IVL) assembly including a progressive spark surface emitter (PSSE) architecture configured for peripheral vascular applications, including compatibility with guidewires between about 0.025-inch (0.635 mm) to about 0.095-inch (2.413 mm), including 0.035-inch (0.889 mm) guidewires, with reference to FIGS. 1-2. The discussion turns to example PSSE module configurations, including pre-encapsulation assemblies, axial cross-sections, and spark-generating region geometry, with reference to FIGS. 3-8. Example windowless and non-linear conductor channel configurations are then described with reference to FIGS. 10-11. The discussion turns to example catheter cross-sectional geometry, multi-emitter longitudinal arrangements, and electrical schematic configurations with reference to FIGS. 12-14. Example catheter assembly architecture for peripheral use is described with reference to FIG. 15. An example emission cycle plot illustrating electrical and operational parameters over successive discharge events is described with reference to FIG. 16. Example methods of designing and operating a PSSE-based IVL assembly are described with reference to FIGS. 17-18. The discussion then turns to example co-radial electrode geometry, spark initiation radius relationships, exposed discharge surface area relationships, and spark gap geometric relationships with reference to FIGS. 19A-26. Example shaped conductor channel configurations, non-linear channel geometry, and curvilinear channel boundary configurations are described with reference to FIGS. 27A-30B. Finally, an example method of manufacturing an emitter module is described with reference to FIG. 31, and various additional embodiments and features are discussed in the sections that follow.

Calcified lesions within peripheral blood vessels, such as the superficial femoral artery (SFA), common femoral artery (CFA), and iliac arteries, may present significant challenges for interventional procedures. Calcium deposits within vessel walls may resist conventional balloon dilation and may complicate delivery and deployment of endovascular devices such as stents and stent grafts. Peripheral procedures may involve transitioning between vessel preparation and delivery of large-profile endovascular devices, and such transitions may involve guidewire exchanges when an IVL catheter is configured only for smaller guidewire platforms, such as 0.014-inch (0.356 mm) or 0.018-inch (0.457 mm) guidewires. Guidewire exchanges may increase procedural time and fluoroscopy exposure, and may introduce risk of losing wire position in heavily calcified or tortuous anatomy. Loss of secure access in large-diameter vessels may have serious clinical consequences, particularly when rapid transition to bailout therapies has to be performed. As of the date of this application, applicant is not aware of any commercially available IVL systems capable of use with 0.035-inch (0.889 mm) guidewires or guidewires of between 0.025 inch (0.635 mm) to 0.095 inch (2.413 mm), or between about 0.025 inch (0.635 mm) to about 0.095 inch (2.413 mm).

FIG. 1 depicts an example embodiment of an intravascular lithotripsy (IVL) assembly 100 including a progressive spark surface emitter (PSSE) architecture, an emitter body peripherally tangential conductor arrangement, and an intra-catheter conductor configuration. The IVL assembly 100 may include a proximal interaction module 102. The proximal interaction module 102 may, for example, be coupled to a catheter 104. The proximal interaction module 102 may, for example, fluidly couple accessories (e.g., syringes, intravenous (IV) tubing) to the catheter 104. The proximal interaction module 102 may, for example, couple to a control module (e.g., for catheter steering, inflation/deflation, electrical therapy, and/or electrical sensing). The proximal interaction module 102 may, for example, be configured as a manual interface for a physician to manipulate the catheter 104. The catheter 104 may extend along a longitudinal axis to a distal end 108. The catheter 104 may, for example, be configured to navigate through vascular pathways, including peripheral vessels. The catheter 104 and/or a balloon 106 may, for example, be coated, such as with a hydrophilic coating. A hydrophilic coating may, for example, induce self-moisturization during operation.

The catheter 104 may include an inner shaft 114 disposed within the catheter 104. The inner shaft 114 may extend longitudinally through the balloon 106. The inner shaft 114 may define or surround a guidewire lumen. In some embodiments, the guidewire lumen may be sized to accommodate relatively large guidewires, including guidewires having diameters of between 0.025-inch (0.635 mm) to 0.095-inch (2.413 mm), or between about 0.025-inch (0.635 mm) to about 0.095-inch (2.413 mm), including 0.035-inch (0.889 mm) guidewires, approximately 0.035 inch (0.889 mm) guidewires, or larger guidewires. Such configurations may be particularly suitable for peripheral and large-bore access procedures, including preparation of iliac and femoral vessels for delivery of large-profile endovascular devices. The ability to accommodate a 0.035-inch (0.889 mm) guidewire may, for example, reduce or eliminate guidewire exchange when transitioning between vessel preparation and large-profile device delivery, potentially reducing procedural time, fluoroscopy exposure, and risk of losing wire position in heavily calcified or tortuous anatomy.

The inner shaft 114 may include one or more conductors 112. The conductors 112 may, for example, be routed along peripheral regions of the inner shaft 114 and/or embedded within wall structures of the inner shaft 114, such that conductor routing does not occupy the guidewire lumen space. Such intra-catheter conductor routing may, for example, preserve radial space for the guidewire lumen while accommodating the conductor count associated with multiple emitter modules. In some embodiments, the conductors 112 may be arranged in a circumferential pattern around the inner shaft 114 and may remain within a radial envelope defined by the catheter 104. This arrangement may, for example, allow increased conductor count without a proportional increase in overall catheter diameter.

The balloon 106 may be disposed about the catheter 104. The balloon 106 may surround at least a portion of the inner shaft 114. The balloon 106 may, for example, be inflatable to contact a vessel wall and establish a fluid-filled environment around one or more emitter modules 116 disposed along the inner shaft 114. The balloon 106 may have a length and diameter selected for treatment of target vessels. The balloon 106 may, for example, be configured to deliver lithotripsy energy over an extended treatment zone in peripheral applications. In some embodiments, the balloon 106 may have a length greater than that used in coronary applications.

Emitter modules 116 may be disposed along the inner shaft 114, either partially or completely within the balloon 106. The emitter modules 116 may be electrically coupled to one or more of the conductors 112. The one or more conductors 112 may electrically couple the emitter modules 116 to one another and to a voltage source and/or control circuitry. Each emitter module 116 may, for example, be configured as a progressive spark surface emitter (PSSE). The emitter module 116 may include an emitter body surface 118 and a conductor surface 120 that may be positioned to define a spark-generating region. When an appropriate voltage is applied to the emitter module 116 via one or more of the conductors 112, an electrical discharge may be generated between the emitter body surface 118 and the conductor surface 120. The electrical discharge may produce a spark that may rapidly vaporize fluid within the balloon 106, thereby generating a pressure pulse. The pressure pulse may propagate away from the emitter module 116 and through the balloon 106. When the IVL assembly 100 is positioned within a blood vessel, the pressure pulse may fracture and/or otherwise modify calcified tissue within and/or adjacent to the vessel wall.

In this example, the emitter body surface 118 may be arranged along a linear or generally linear path that may extend parallel or generally parallel to the longitudinal axis of the inner shaft 114. During repeated emission cycles, material from the conductor surface 120 may be consumed and/or eroded. As erosion progresses, the effective location of the conductor surface 120 may change along the length of the emitter body surface 118, which may cause the spark location to migrate progressively along the emitter body surface 118. The geometry and alignment of the emitter body surface 118 may be selected such that the spark gap between the emitter body surface 118 and the conductor surface 120 may remain constant or substantially constant despite this migration. Maintaining a constant or substantially constant spark gap may, for example, facilitate reliability of spark formation and consistency of spark characteristics, such as timing and energy, across multiple emission cycles. Consistency of spark characteristics may, in turn, facilitate consistency and predictability of lithotripsy therapy delivered by the IVL assembly 100.

In some embodiments, the emitter body surface 118 and the conductor surface 120 may be positioned at a common radial distance from the longitudinal centerline of the inner shaft 114, such that an arcuate surface concentric with the longitudinal centerline passes through both the emitter body surface 118 and the conductor surface 120. This co-radial arrangement may, for example, contribute to compact radial packaging of the emitter module 116, potentially facilitating accommodation of the guidewire lumen within the same outer catheter profile. In some embodiments, an encapsulating dielectric material may separate the emitter body from the conductor such that electrical discharge between the emitter body and the conductor is physically inhibited except between the emitter body surface 118 and the conductor surface 120. This structural constraint may, for example, cause the location of electrical discharge to be determined by geometry rather than by random breakdown processes.

The IVL assembly 100 may include one or more radiopaque markers 122 disposed along the catheter 104 at and/or near locations corresponding to the emitter modules 116 and/or ends of a treatment region. The radiopaque markers 122 may, for example, facilitate visualization of the catheter 104 and emitter array under fluoroscopy and/or other imaging modalities, assisting an operator in positioning the emitter modules 116 relative to a target lesion.

The IVL assembly 100 may, for example, be configured with a crossing profile selected to be compatible with target body structures. In some embodiments, the crossing profile may be less than or equal to 0.085 inches (2.159 mm), such as to fit within a 7 French or smaller introducer sheath. In some embodiments, the crossing profile may be less than or equal to 0.065 inches (1.651 mm), such as to fit within a 6 French or smaller introducer sheath. In some embodiments, the crossing profile may be less than or equal to 0.061 inches (1.550 mm), such as to fit within a 5 French or smaller introducer sheath. The combination of a PSSE architecture with a guidewire lumen sized for a guidewire between 0.025-inch (0.635 mm) to 0.095-inch (2.413 mm), or between about 0.025-inch (0.635 mm) to about 0.095-inch (2.413 mm), or a 0.035-inch (0.889 mm) guidewire, may be particularly advantageous in that it may reduce or avoid guidewire exchange, while preserving crossing profile, flexibility, and electrical performance characteristics.

IVL systems configured for peripheral applications may benefit from compatibility with larger-diameter guidewires, such as guidewires between 0.025-inch (0.635 mm) to 0.095-inch (2.413 mm), or between about 0.025-inch (0.635 mm) to about 0.095-inch (2.413 mm), including 0.035-inch (0.889 mm) guidewires, which may provide enhanced support and stability during navigation through larger peripheral vessels. Peripheral procedures may involve transitioning between vessel preparation and delivery of large-profile endovascular devices, and such transitions may involve guidewire exchanges when the IVL catheter is configured only for smaller guidewire platforms. Guidewire exchanges may increase procedural time and fluoroscopy exposure, and may introduce risk of losing wire position in heavily calcified or tortuous anatomy. Accommodating a larger guidewire lumen within an IVL catheter presents significant engineering challenges, as the guidewire lumen, high-voltage conductors, insulating structures, and emitter components are all to be packaged within a limited radial cross-section. Balancing guidewire compatibility, electrical safety, emitter performance, and catheter profile may present significant engineering challenges. The PSSE architectures disclosed herein may facilitate integration of IVL emitters within catheter systems that accommodate larger guidewire lumens while maintaining acceptable crossing profiles, flexibility, and electrical performance.

FIG. 2 depicts an enlarged view of a portion of an intravascular lithotripsy (IVL) catheter assembly 200, illustrating example relative positioning of a balloon 206, an inner shaft 214, one or more emitter modules 216, and internal conductors. The IVL catheter assembly 200 may include features described in relation to any other embodiment disclosed herein. The intravascular lithotripsy (IVL) catheter 200 may be an example implementation including the catheter 104 described with reference to FIG. 1. In the illustrated embodiment, the balloon 206 is disposed around a portion of a catheter 204. The balloon 206 may define an interior volume that may be filled with fluid during use. The balloon 206 may be coupled to the catheter 204 at proximal and distal ends, which may define a treatment region in which one or more emitter modules 216 are positioned. The balloon 206 may be configured for inflation to contact a peripheral vessel wall. The balloon 206 may be configured to establish a fluid medium suitable for propagation of pressure waves generated by the emitter modules 216.

The inner shaft 214 extends longitudinally through the balloon 206 and may define or surround a guidewire lumen. The guidewire lumen may be sized to accommodate relatively large guidewires, including guidewires having diameters of between 0.025-inch (0.635 mm) to 0.095-inch (2.413 mm), or between about 0.025-inch (0.635 mm) to about 0.095-inch (2.413 mm), including approximately or equal to 0.035 inch (0.889 mm), or larger. Other guidewire sizes are also possible. In some embodiments, the inner shaft 214 may include material selections, wall thicknesses, and/or structural reinforcements that may permit accommodation of such guidewire diameters while maintaining flexibility and resistance to kinking. The inner shaft 214 may further serve as a structural support for the emitter modules 216 and for routing of electrical conductors.

One or more conductors may extend along the inner shaft 214. The conductors in this example include one or more supply conductors 212. The conductors in this example include one or more return conductors 232. The supply conductors 212 may, for example, deliver electrical energy from a control module and/or pulse generator to respective emitter modules 216 disposed along the inner shaft 214. The return conductor 232 may, for example, provide a return current path to close the discharge circuit for each spark event. For example, the return conductor 232 may also be referred to as a ground conductor. The conductors 212 and 232 may be routed in a manner that preserves radial space for the guidewire lumen. For example, some embodiments (such as depicted) may position the conductors 212, 232 along peripheral regions of the inner shaft 214 and/or embed the conductors 212, 232 within wall structures of the catheter 204. Such conductor routing may, for example, reduce radial crowding within the catheter 204 and/or facilitate use of larger guidewire diameters relative to catheter architectures in which conductors occupy a central and/or shared lumen region.

In some embodiments, the spatial relationship between the inner shaft 214, the emitter modules 216, and the conductors 212 and 232 may offer advantages over other IVL catheter architectures such as, by way of example and not limitation, architectures that rely on centrally located conductors and/or fixed electrode bodies. For example, the illustrated arrangement may permit electrical conductors to be routed longitudinally alongside the guidewire lumen rather than through it. Such routing may, for example, enable compatibility with larger guidewires, such as without a proportional increase in catheter outer diameter. The guidewire lumen diameter may, for example, be decoupled from conductor count. For example, radial positioning of conductors around the inner shaft 214 may advantageously enable decoupling, such as rather than by lumen sharing. An increased number of conductors may, for example, advantageously be accommodated without a proportional increase in overall catheter diameter.

The emitter module 216 includes, in this example, an emitter body 217. The emitter body 217 may be disposed around the inner shaft 214, as depicted. The emitter body 217 may, for example, be disposed adjacent the inner shaft 214. The emitter body 217 may, for example have one or more exposed regions configured to participate in spark generation. In the illustrated embodiment, the emitter module 216 may be positioned such that its outer profile remains compact relative to the inflated balloon 206, which may facilitate maintaining a low crossing profile even when accommodating a larger guidewire lumen. The emitter module 216 may, for example, be electrically coupled to one or more of the supply conductors 212 and the return conductors 232, such as to form part of a discharge circuit. When an appropriate voltage is applied, electrical discharge may be generated at the exposed regions of the emitter module 216. The discharge may, for example, produce a pressure pulse that propagates through the fluid-filled balloon 206 and into adjacent vessel tissue.

In some embodiments, the emitter module may be configured as a progressive spark surface emitter (PSSE), in which spark initiation locations migrate along a predetermined spark progression path, such as, for example, electrode material erodes during repeated discharge events. Such configurations may, for example, distribute erosion over a larger electrode surface area, which may advantageously extend emitter operational life while maintaining consistent pressure wave output. The compact radial arrangement of the conductors 212 and 232 relative to the inner shaft 214 may, for example, allow the emitter module to be positioned within the same radial envelope as the conductors. This compact arrangement may, for example, reduce step changes in profile along the catheter 204 and/or facilitate smooth advancement through peripheral anatomy.

In this example, one or more markers 222 are disposed along the catheter 204. For example, markers may be disposed at and/or near a longitudinal position corresponding to the emitter module 216 and/or a balloon treatment region. The marker 222 may be configured as a band and/or other feature, as shown. The marker 222 may, for example, include radiopaque and/or otherwise imageable material. The marker 222 may, for example, facilitate visualization of the catheter assembly 200 in vivo and/or may assist with positioning of the balloon 206 and emitter module 216 relative to a target treatment site (e.g., under fluoroscopy or other imaging modalities).

FIG. 3 depicts perspective views of an example progressive spark surface emitter (PSSE) module 316 shown in different orientations relative to an inner shaft. The PSSE module 316 may be an example implementation of the emitter module 116 described with reference to FIG. 1. The different orientations illustrated in FIG. 3 may, for example, assist in visualizing the three-dimensional spatial relationships between the emitter body, conductor, and capsule components of the PSSE module 316.

The PSSE module 316 may include a capsule 301 that may be disposed circumferentially around a portion of the inner shaft. The capsule 301 may be an example implementation of the encapsulating dielectric material described with reference to the emitter modules 116 of FIG. 1. The inner shaft may be an example implementation of the inner shaft 114 described with reference to FIG. 1. The inner shaft may define or surround a guidewire lumen, which may be sized to accommodate relatively large guidewires, including guidewires having diameters of between 0.025-inch (0.635 mm) to 0.095-inch (2.413 mm), or between about 0.025-inch (0.635 mm) to about 0.095-inch (2.413 mm), including approximately or equal to 0.035 inch (0.889 mm), or other sizes as disclosed herein. The capsule 301 may at least partially encapsulate conductive components of the PSSE module 316. The capsule 301 may be formed of a dielectric and/or otherwise electrically insulative material. In some embodiments, the capsule 301 may be formed by coating, potting, over-molding, and/or combinations thereof. The capsule 301 may electrically isolate conductive elements while permitting selected conductive regions to remain exposed for spark generation. The capsule 301 may provide mechanical support and/or positional stability, such as for conductive elements relative to the inner shaft.

As shown, one or more conductors 312 may extend longitudinally along the inner shaft and may enter and/or pass adjacent to the PSSE module 316. The conductors 312 may be an example implementation of the conductors 112 described with reference to FIG. 1 and the conductors 212 described with reference to FIG. 2. The conductors 312 may be configured to deliver electrical energy to and/or from the PSSE module 316. In some embodiments, the conductors 312 may be routed along peripheral regions of the inner shaft rather than occupying a central lumen region. Such routing may, for example, preserve radial space within the catheter assembly and may facilitate inclusion of a guidewire lumen sized for 0.035-inch (0.889 mm) guidewires, guidewires between 0.025-inch (0.635 mm) to 0.095-inch (2.413 mm), or between about 0.025 inch (0.635 mm) and about 0.095 inch (2.413 mm), or other large sizes, such as without a corresponding increase in catheter outer diameter. In some embodiments, the catheter may be as small as a 4-5 French catheter while still accommodating such guidewire lumen dimensions.

The emitter body surface 318 may be an example implementation of the emitter body surface 118 described with reference to FIG. 1. The conductor surface 320 may be an example implementation of the conductor surface 120 described with reference to FIG. 1. The emitter body surface 318 may be arranged relative to the conductor surface 320, defining a spark-generating region. In the illustrated embodiment, the emitter body surface 318 and the conductor surface 320 may be positioned at the same or substantially the same radial distance from the longitudinal axis of the inner shaft, for example, such that an arcuate surface concentric with the longitudinal axis passes through both the emitter body surface 318 and the conductor surface 320. This co-radial arrangement may, for example, contribute to compact radial packaging of the PSSE module 316, such as further described herein. The capsule 301 may bound the emitter body surface 318 and the conductor surface 320 such that electrical discharge between the emitter body and the conductor is physically inhibited except at the defined spark-generating region, such as further described herein.

In some embodiments, the conductor 312 may be routed such that its outermost surface is flush or substantially flush with, recessed relative to, and/or otherwise does not extend radially beyond the outer periphery defined by the emitter body surface 318. Such configurations may, for example, allow the PSSE module 316 to define a maximum radial profile for both the emitter body surface 318 and the associated conductor surface 320. Routing the conductor 312 in this manner may, for example, reduce or avoid localized radial protrusions that could otherwise increase a maximum outer diameter of the PSSE module 316. This compact radial packaging may be advantageous for catheter systems that include larger guidewire lumens, including those configured for 0.035-inch (0.889 mm) or other large size guidewires, as further described herein.

In some embodiments, the PSSE module 316 may be configured such that spark initiation locations migrate along a predetermined spark progression path as electrode material erodes during repeated discharge events, as further described herein. The capsule 301 may bound the exposed discharge surfaces such that erosion is guided along the defined path rather than concentrating at a single location. Such configurations may, for example, distribute material loss over a larger electrode surface area, which may, for example, advantageously extend emitter operational life and/or maintain more consistent pressure wave output across successive discharge cycles. In some embodiments, conductive material deposited between the emitter body surface 318 and the conductor surface 320 during repeated discharge events—e.g., a “slag bridge”—may be guided along the predetermined spark progression path by the bounding geometry of the capsule 301, such that refuse (e.g., carbon) accumulation does not cause a fixed short-circuit between the emitter body and the conductor at a single location.

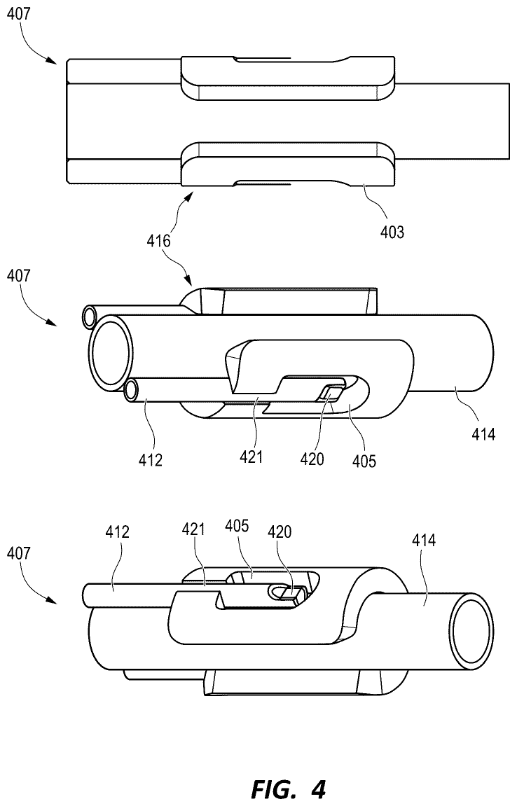

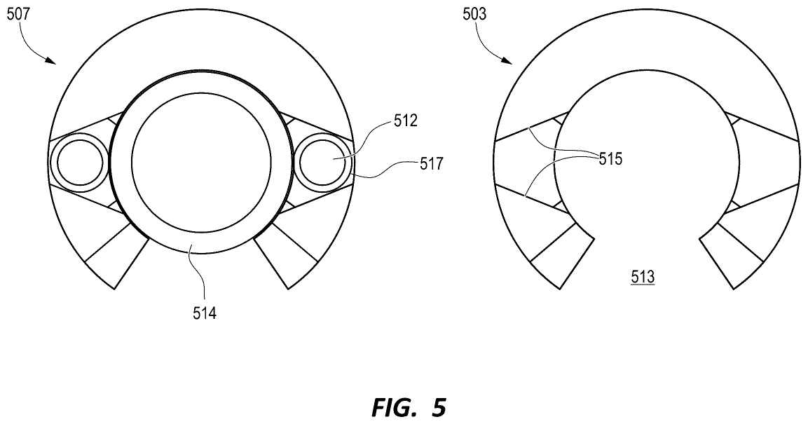

The different orientations of the PSSE module 316 illustrated in FIG. 3 show the emitter body surface 318 and the conductor surface 320 from multiple viewing angles, illustrating how the spark-generating region is defined by the geometry of the capsule 301 and the relative positioning of the emitter body and conductor. In some embodiments, the capsule 301 may include one or more exposed regions formed by selective removal and/or inhibition of insulating material, such as by laser ablation, mechanical skiving, and/or masking during application of the capsule material, such as further described with reference to FIG. 31. The pre-capsulation assembly of the PSSE module 316 is further illustrated in FIG. 4, and the axial cross-sectional geometry of the emitter body and inner shaft is further illustrated in FIG. 5.

FIG. 4 depicts example pre-capsulation configurations 407 of a progressive spark surface emitter (PSSE) module 416 shown in multiple views and assembly stages. The PSSE module 416 may be an example implementation of the emitter module 116 described with reference to FIG. 1 and the emitter module 216 described with reference to FIG. 2. The multiple views illustrated in FIG. 4 may, for example, represent successive orientations and/or assembly steps of the PSSE module 416 prior to application of a capsule, such as the capsule 301 described with reference to FIG. 3.

The pre-capsulation configuration 407 includes an emitter body 403 depicted assembled over an inner shaft 414. The inner shaft 414 may be an example implementation of the inner shaft 114 described with reference to FIG. 1 and the inner shaft 214 described with reference to FIG. 2. The inner shaft 414 may define and/or surround a guidewire lumen that may be sized to accommodate relatively large guidewires, including guidewires having diameters of between 0.025-inch (0.635 mm) to 0.095-inch (2.413 mm), or between about 0.025 inch (0.635 mm) and about 0.095 inch (2.413 mm), approximately or equal to 0.035 inch (0.889 mm), or even larger, as disclosed herein. In some embodiments, the inner shaft 414 may include material selections, wall thicknesses, and/or structural reinforcements that may permit accommodation of such guidewire diameters while maintaining flexibility and resistance to kinking, such as described with reference to FIG. 2.

The emitter body 403 may include one or more discharge apertures 405 formed through a wall of the emitter body 403. The discharge aperture 405 may be configured to receive a portion of a conductor 412 and/or to expose one or more emitter body surfaces that may participate in spark generation. The discharge aperture 405 may be an example implementation of the discharge aperture described with reference to the emitter body surfaces 318 of FIG. 3. One or more conductors 412 may extend longitudinally along the inner shaft 414. The one or more conductors 412 may be positioned such that conductor surfaces 420 are disposed within the discharge aperture 405. The conductors 412 may be an example implementation of the conductors 112 described with reference to FIG. 1, the conductors 212 described with reference to FIG. 2, and/or the conductors 312 described with reference to FIG. 3. The conductor surface 420 may be an example implementation of a conductor surface described with reference to the conductor surface 320 of FIG. 3.

In some embodiments such as depicted, the conductors 412 may be routed through one or more conductor channels 421 formed in the emitter body 403. The conductor channels 421 may guide the conductors 412 into alignment with the discharge aperture 405 and/or may assist in positioning the conductor surfaces 420 relative to exposed emitter body surfaces. The conductor channels 421 may be an example implementation of the conductor channel geometry described with reference to the emitter body 302 of the provisional application. Some embodiments may, for example, include cutouts. Cutouts may, for example, advantageously allow for wire routing to and/or from distal emitters to be flush with the proximal emitters. Such embodiments may, for example, reduce a crossing profile of the IVL assembly 100 described with reference to FIG. 1.

The emitter body 403 may further include one or more conductor retaining surfaces or features that may retain the conductors 412 in a desired radial and/or longitudinal position relative to the emitter body 403 and/or the inner shaft 414. Such retaining features may, for example, advantageously assist in maintaining alignment of the conductors 412 within the discharge aperture 405 during assembly and/or subsequent operation. In some embodiments, the retaining features may advantageously permit (e.g., partial) capture of the conductors 412 by the emitter body 403, rather than fully enclosing the conductors 412. Partial capture may, for example, advantageously allow portions of the conductors 412 to remain flush with and/or near an outer periphery of the emitter body 403. Such partial capture may, for example, retain the conductors 412 within the conductor channels 421, such as even without adhesive bonding and/or additional external support members.

In some embodiments, a wire configuration including bridge wire(s) and/or conductor channels 421 such that the conductors 412 are flush with the outer periphery of the emitter body 403 may advantageously enable increased emitter body wall thickness. Increased wall thickness may, for example, increase the emitter body's resistance to erosion during repeated spark generation events, potentially increasing durability and/or longevity of the emitter body 403 over multiple emission events. In some embodiments, the emitter body 403 may include a longitudinal through-channel that may permit conductors associated with emitter modules disposed distally along the catheter to pass longitudinally along the inner shaft 414 while remaining radially within the outer periphery of the emitter body 403. Such embodiments may, for example, enable reduced crossing profiles of the IVL assembly 100 described with reference to FIG. 1, and/or increased longevity.

In some embodiments, spark generation within the discharge aperture 405 may involve controlled concentration of electrical charge at limited surface areas of the conductor surfaces 420 and opposing emitter body surfaces. A specific geometry, such as a small emitter body surface area, may, for example, focus the charge concentration on a target region. A smaller emitter body surface and/or conductor surface 420 may, for example, reduce rise time of a spark formation event. Without being bound to a particular theory, a smaller surface area may reduce the time to build sufficient charge concentration on opposing conductive surfaces of a spark gap to induce a gaseous bridge across the spark gap. Application of a high-voltage pulse may cause localized charge accumulation at the conductor surface 420 and an opposing charge accumulation at the exposed emitter body surface. As electrical energy accumulates within the fluid medium surrounding the PSSE module 416, localized heating and vaporization may occur, which may lead to formation of a gaseous discharge across a spark gap. Ionization of the intervening medium may permit an electrical arc to propagate between the conductor surface 420 and the emitter body surface. Formation of the arc may produce erosion of the conductor 412, emitter body 403, and/or capsule material over repeated emission cycles.

Once formed, the spark may generate a rapidly expanding pressure wave that may propagate away from the emitter body surface and into surrounding fluid, where it may interact with adjacent structures such as calcified tissue within a blood vessel. In some embodiments, the geometry of the emitter body 403, discharge aperture 405, and conductor placement may be selected to promote controlled and repeatable spark formation. For example, the discharge aperture 405 may be configured such that a first edge-to-edge distance between the conductor surface 420 and the opposing emitter body surface is less than a second, median electrode distance between median spark initiation sites of the respective surfaces. This geometric hierarchy may, for example, cause electrical discharge to initiate preferentially at the nearest spark initiation sites before propagating to median sites at higher applied voltages. Such geometric control of spark initiation may, for example, reduce variability in pressure wave generation and reduce dissipation of electrical energy.

In some embodiments, the conductor surface 420 and the emitter body surface within the discharge aperture 405 may be configured such that an exposed combined surface area of the emitter body surfaces is substantially similar to an exposed surface area of the corresponding conductor surface 420. Such surface-area relationships may, for example, promote efficient electrical discharge behavior and/or may contribute to consistent spark formation during repeated emission cycles, as described with reference to the emitter module 116 of FIG. 1 and the PSSE module 316 of FIG. 3.

After assembly of the emitter body 403 and conductors 412 over the inner shaft 414, a capsule, such as the capsule 301 described with reference to FIG. 3, may subsequently be disposed about the assembly. The capsule may be formed of a dielectric and/or otherwise electrically insulative material. The capsule may encapsulate portions of the emitter body 403 and conductors 412 while leaving selected conductive regions exposed at the discharge aperture 405. In the finished configuration, exposed conductor surfaces 420 and exposed emitter body surfaces may define a spark-generating region when a high-voltage pulse is applied. In some embodiments, the exposed surfaces may be defined by selective removal of encapsulating material, for example by laser ablation, mechanical skiving, and/or masking during application of the capsule material.

Some embodiments, including the configuration depicted in FIG. 4, may permit conductor routing that remains partially or fully within a radial envelope defined by an outer periphery of the emitter body 403. For example, the conductor channels 421 and retaining features may allow conductors 412 to pass longitudinally along the inner shaft 414 without protruding radially beyond the outer surface of the emitter body 403. Such routing may, for example, reduce a crossing profile of the IVL assembly 100 described with reference to FIG. 1. This compact radial packaging may be advantageous for catheter systems that include larger guidewire lumens, including those configured for 0.035-inch guidewires (0.889 mm), guidewires between 0.025-inch (0.635 mm) to 0.095-inch (2.413 mm), or between about 0.025 inch (0.635 mm) and about 0.095 inch (2.413 mm), or other large sizes, as it may reduce or avoid increases in overall catheter diameter while maintaining emitter durability and electrical isolation, as described with reference to FIGS. 1-3.

FIG. 5 depicts axial views of example emitter body configurations of a PSSE module, illustrating internal lumen geometry and conductor positioning. The configurations illustrated in FIG. 5 may be example implementations of the emitter body 403 and inner shaft 414 described with reference to FIG. 4, shown from an axial perspective to illustrate geometric relationships not visible in the longitudinal views of FIGS. 3 and 4.

In the embodiment shown on the left, a pre-encapsulated assembly 507 of an emitter module is illustrated. The pre-encapsulated assembly 507 may include an emitter body 503 disposed circumferentially around an inner shaft 514. The inner shaft 514 may be an example implementation of the inner shaft 114, 214, and/or 414 described with reference to FIGS. 1, 2, and 4, respectively. The inner shaft 514 may define or surround a guidewire lumen. The inner shaft 514 may include one or more shaft channels 513. The shaft channels 513 may extend longitudinally along the inner shaft 514. The shaft channel 513 may be configured to route one or more conductors 512 while preserving radial space for the guidewire lumen. Such shaft channel arrangements may, for example, facilitate accommodation of relatively large guidewires, including guidewires having diameters of between 0.025-inch (0.635 mm) to 0.095-inch (2.413 mm), or between about 0.025 inch (0.635 mm) and about 0.095 inch (2.413 mm), approximately or equal to 0.035 inch (0.889 mm) or even larger, such as without a proportional increase in catheter outer diameter.

As shown, one or more conductors 512 may be positioned within and/or adjacent to the shaft channel 513. The conductors 512 may include an electrically conductive core surrounded by conductor insulation. The conductor insulation may be an example implementation of the conductor insulation described with reference to the conductors 112, 212, 312, and/or 412 of FIGS. 1-4. The conductor insulation may electrically isolate the conductor 512 from surrounding structures. The conductor insulation may be selectively removed and/or modified at spark-generating regions, such as described with reference to FIGS. 3 and 4. In some embodiments, the conductors 512 may be positioned such that exposed conductor surfaces remain within a radial envelope defined by an outer periphery of the emitter body 503.

The emitter body 503 may include one or more conductor retaining surfaces 515. The conductor retaining surfaces 515 may be an example implementation of the conductor retaining features described with reference to the emitter body 403 of FIG. 4. The conductor retaining surfaces 515 may be configured to engage, support, and/or constrain the conductors 512 in a desired radial and circumferential position relative to the emitter body 503. Such retaining surfaces may, for example, assist in maintaining alignment between conductor surfaces and corresponding emitter body surfaces during assembly and during repeated emission cycles. In some embodiments, the conductor retaining surfaces 515 may permit partial capture of the conductors 512 while allowing portions of the conductors to remain flush or near-flush with the outer profile of the emitter body 503, such as described with reference to FIG. 4.

In the embodiment shown on the right, the emitter body 503 is illustrated without conductors installed, exposing internal geometry associated with conductor retention and spark-participating surfaces. The conductor retaining surfaces 515 may be more clearly visible in this view and may define regions where conductors may be seated prior to encapsulation. The emitter body 503 may further include features configured to cooperate with a subsequently applied capsule, such as the capsule 301 described with reference to FIG. 3, which may encapsulate portions of the emitter body 503 and conductor insulation 517 while leaving selected conductive regions exposed.

As illustrated in both views of FIG. 5, conductors 512 may be routed along the inner shaft 514 through shaft channels 513 and retained by conductor retaining surfaces 515 without protruding radially beyond the outer periphery of the emitter body 503. This compact radial arrangement may, for example, reduce step-ups in profile along the catheter and may be advantageous for IVL catheter systems that include larger guidewire lumens. In addition, maintaining conductor placement within the radial envelope of the emitter body 503 may permit increased wall thickness in portions of the emitter body 503, which may provide additional material to accommodate progressive erosion during repeated spark generation events, such as described with reference to FIGS. 3 and 4.

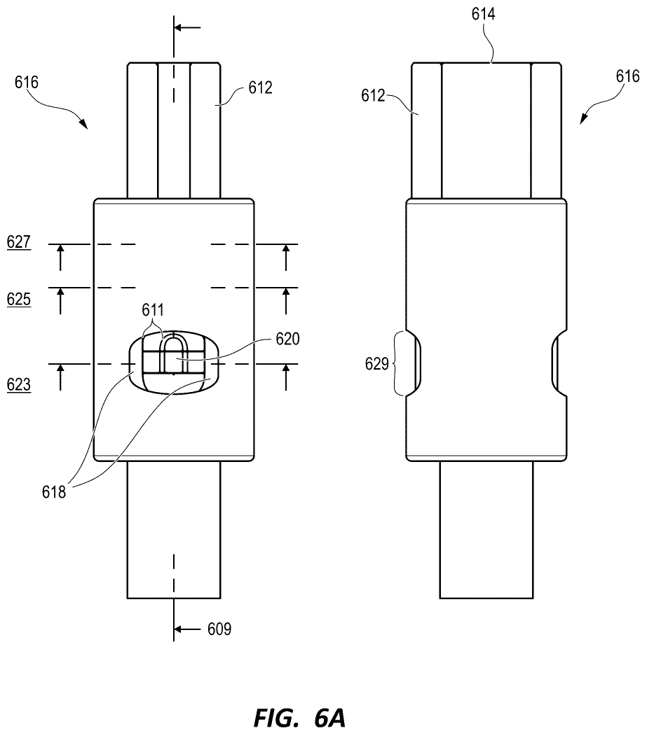

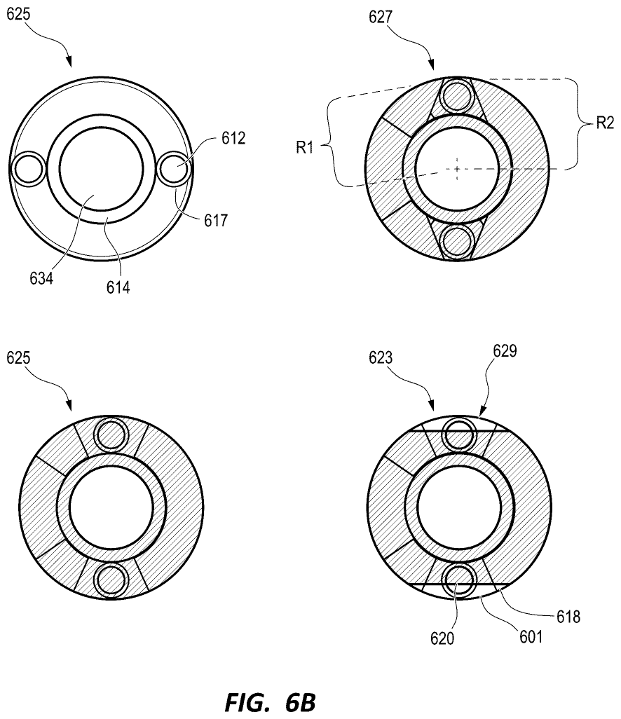

FIG. 6A depicts longitudinal plan views of an example progressive spark surface emitter (PSSE) module 616. The PSSE module 616 may be an example implementation of the emitter module 116 described with reference to FIG. 1 and/or the PSSE module 316 described with reference to FIG. 3. The views illustrated in FIG. 6A represent different longitudinal orientations of the same PSSE module 616 positioned along an inner shaft of an intravascular lithotripsy catheter. The PSSE module 616 extends along or generally along a longitudinal axis 609.

As shown in FIG. 6A, the PSSE module 616 may include exposed conductor regions 629 (e.g., exposed metal regions) disposed about the PSSE module 616. Each exposed conductor region 629 may correspond to a localized region along the PSSE module 616 where electrical field intensity and/or charge density may increase during application of a high-voltage pulse. In some embodiments, such as shown, each exposed conductor region 629 may include a conductor surface 620 positioned adjacent to a corresponding emitter body surface 618, with a spark gap 611 defined therebetween. The conductor surface 620 and emitter body surface 618 may be example implementations of the conductor surface 120 and emitter body surface 118 described with reference to FIG. 1.

The spacing of the exposed metal regions 629 may allow spark generation to occur at multiple positions and/or may support progressive migration of an effective spark location during repeated emission cycles. As conductive material is consumed/eroded during repeated discharge events, the effective spark location may shift along the longitudinal axis 609 while a controlled or substantially controlled spark gap 611 may be maintained. Such longitudinally shifting geometry may support consistent spark formation and/or pressure-wave generation over multiple pulses. In some embodiments, conductive material deposited between the emitter body surface 618 and the conductor surface 620 during repeated discharge events—for example, a slag bridge—may be guided along a predetermined spark progression path by the bounding geometry of the capsule, such that refuse accumulation does not cause a fixed short-circuit between the emitter body and the conductor at a single location, as described with reference to FIG. 3.

FIG. 6B depicts a series of axial cross-sectional views taken at different longitudinal positions of the PSSE module 616 shown in FIG. 6A. The axial views of FIG. 6B may illustrate different functional regions of the PSSE module 616 and their respective geometric relationships. An inner shaft 614 may be shown in each axial view, defining or surrounding a guidewire lumen 634. The inner shaft 614 may be an example implementation of the inner shaft 114 described with reference to FIG. 1 and the inner shaft 514 described with reference to FIG. 5.

The axial view labeled 625 depicts an emitter body cross-section. In this view, the emitter body is disposed circumferentially around the inner shaft 614. The geometry illustrated in cross-section 625 may represent a region of the PSSE module 616 in which the emitter body wall thickness is continuous or substantially continuous, without a discharge aperture or exposed conductive region present at that longitudinal position.

The axial view labeled 627 depicts a conductor retainer cross-section. In this region, one or more conductors 612 are positioned relative to the emitter body and constrained by conductor retaining features, such as the conductor retaining surfaces 515 described with reference to FIG. 5. In some embodiments, an emitter radius R1 may correspond to a conductor placement radius R2 in this region. Such radial correspondence may allow both the conductor 612 (e.g., including conductor insulation 617, as shown) and the emitter body to be effectively inscribed within a common radial envelope. In some embodiments, a diameter of the conductor 612 may match a thickness of the emitter body wall. Equivalent or substantially equivalent thicknesses, such as shown in FIG. 6B, may, for example, allow both the conductor and the emitter body to be effectively inscribed in the same circle. This geometric relationship may, for example, facilitate manufacturability. In some embodiments, a larger wire gauge matching the emitter body wall thickness may, for example, enhance energy delivery efficiency. In some embodiments, using a smaller wire gauge matching a thinner emitter body wall thickness may, for example, provide a more compact design.

The axial view labeled 623 may depict an exposed conductor cross-section corresponding to an exposed metal region 629. In this region, a portion of encapsulating material may be removed or thinned to expose a conductor surface 620 and a corresponding emitter body surface 618. The exposed conductor surface 620 and the emitter body surface 618 may be separated by a spark gap 601. This configuration may define a localized exposed metal region 629 where electrical discharge may occur when a high-voltage pulse is applied. Progressing from the emitter body cross-section 625 to the conductor retainer cross-section 627, the conductor retaining surfaces may terminate in the discharge aperture. Progressing to the exposed conductor cross-section 623, a portion of the capsule may be removed to form the charge concentration region, exposing the conductor surface 620 and emitter body surface 618.

In some embodiments, the relative sizing of the conductor 612 and the wall thickness of the emitter body may be selected such that both remain within the same radial envelope across the different axial regions shown in FIG. 6B. Such compact radial packaging may, for example, advantageously facilitate accommodation of a guidewire lumen sized for relatively large guidewires, including guidewires having diameters of between 0.025-inch (0.635 mm) to 0.095-inch (2.413 mm), or between about 0.025 inch (0.635 mm) and about 0.095 inch (2.413 mm), approximately or equal to 0.035 inch (0.889 mm) or even larger, such as without a proportional increase in catheter outer diameter.

Accordingly, FIGS. 6A and 6B together may illustrate how longitudinal spacing of exposed metal regions and axial transitions between emitter body cross-sections, conductor retainer regions, and exposed conductor regions may cooperate to support controlled spark formation, progressive erosion behavior, and compact radial packaging of a PSSE module, including in catheter systems configured to accommodate relatively large guidewires.

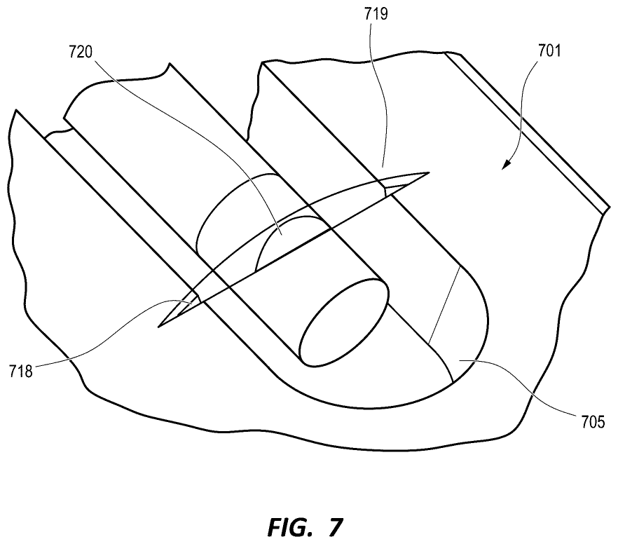

FIG. 7 depicts an enlarged perspective view of an example spark-generating region of a progressive spark surface emitter (PSSE) module. The view illustrates a localized region of the PSSE module in which a conductor and an emitter body surface are arranged relative to a capsule 701 to define a discharge aperture 705 and an associated spark gap.

In the illustrated embodiment, the capsule 701 surrounds at least a portion of an emitter body and one or more conductors. The capsule 701 may be an example implementation of the capsule 301 described with reference to FIG. 3. The capsule 701 is formed of a dielectric and/or otherwise electrically insulative material and encapsulates conductive components while allowing selected conductive regions to be exposed. The discharge aperture 705 is formed through the capsule 701. The discharge aperture 705 may be an example implementation of the discharge aperture 405 described with reference to FIG. 4.

One or more conductors extend adjacent to and/or into the discharge aperture 705. In the illustrated example, a portion of a conductor is exposed within the discharge aperture 705 by way of a cut or skive 719 formed through conductor insulation and/or the capsule 701. The cut or skive 719 removes insulating material from a selected portion of the conductor, exposing a conductor surface 720 at a desired location relative to an exposed emitter body surface 718. The cut or skive 719 may be formed using a mechanical skiving operation, laser ablation, and/or another suitable material-removal process. The geometry of the cut or skive 719 may, for example, be selected to control the exposed surface area of the conductor and/or to influence electric-field concentration within the discharge aperture 705. In some embodiments, the cut or skive 719 may include an angled, tapered, and/or contoured profile that may, for example, promote predictable spark initiation and/or controlled erosion behavior.

The exposed emitter body surface 718 is positioned opposite the exposed conductor surface 720 within the discharge aperture 705. The exposed emitter body surface 718 may be an example implementation of the emitter body surface 618 described with reference to FIG. 6A. The exposed conductor surface 720 and the exposed emitter body surface 718 are separated by a spark gap. During application of a high-voltage pulse, electrical charge may concentrate across the spark gap, which may, for example, lead to ionization of an intervening fluid medium and formation of an electrical discharge.

As spark generation occurs over repeated emission cycles, material at the exposed conductor surface 720 and/or the exposed emitter body surface 718 may be progressively eroded. In some embodiments, the geometry of the discharge aperture 705 and the arrangement of the exposed surfaces may allow the effective spark location to migrate along the exposed emitter body surface 718 as erosion progresses, while maintaining a controlled or substantially controlled consistent spark gap. Such progressive behavior may, for example, distribute erosion over a larger surface area and/or may, for example, contribute to consistent pressure-wave generation over time, as described with reference to FIGS. 3 and 6A.

The discharge aperture 705 and cut or skive 719 are sized and positioned such that the exposed conductive surfaces remain within a radial envelope defined by the capsule 701. Maintaining the exposed conductor and emitter body surfaces within this radial envelope may, for example, contribute to compact radial packaging of the PSSE module.

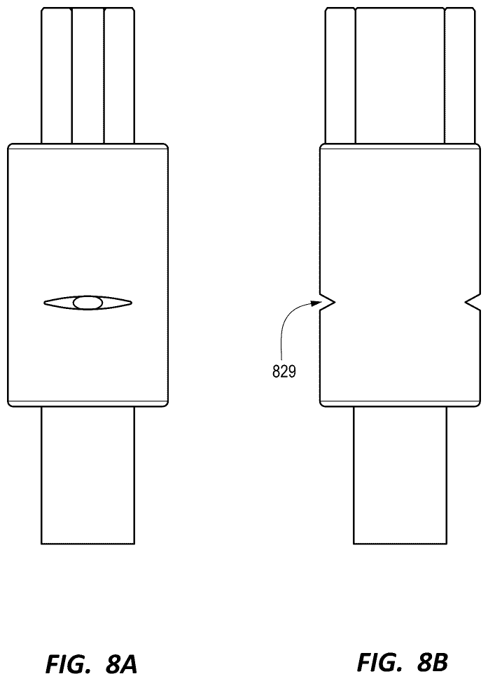

FIGS. 8A-8B depicts an example PSSE module illustrating a narrower initial spark generation geometry. The PSSE module shown in FIGS. 8A-8B may be an example implementation of the emitter module 116 described with reference to FIG. 1 and/or the PSSE module 316 described with reference to FIG. 3. The illustrated views represent different longitudinal sides and/or conditions of the same PSSE module.

An exposed conductor region 829 is illustrated along an exterior of the PSSE module, in both a top view (FIG. 8A) and a right-side view (FIG. 8B). The exposed conductor region 829 may be an example implementation of the exposed metal regions 629 described with reference to FIG. 6A. The exposed conductor region 829 corresponds to a localized region where encapsulating material is thinned, removed, and/or otherwise modified to expose conductive surfaces. In this example, the charge concentration region is formed with a wedge geometry. The wedge geometry may, for example, be created by a superficial cut removing the capsule and conductor insulation, such as the cut or skive 719 described with reference to FIG. 7. Such a wedge geometry may, for example, provide a narrower initial spark generation region relative to embodiments in which a full body of capsule material is removed, such as described with reference to FIG. 6A.

The exposed conductor region 829 exposes a conductor surface (e.g., metallic) and a corresponding emitter body surface that are separated by one or more spark gaps. The exposed conductor surface and exposed emitter body surface at the exposed conductor region 829 are positioned such that electrical charge density and/or electric field intensity may, for example, increase locally during application of a high-voltage pulse. When sufficient voltage is applied, electrical discharge may occur across the corresponding spark gaps, which may, for example, result in spark formation and generation of a pressure wave.

In some embodiments, the geometry and longitudinal extent of the exposed metal region 829 may, for example, be selected to control where spark initiation occurs and/or to influence how the effective spark location migrates during repeated emission cycles. As conductive material is consumed and/or eroded during repeated discharge events, the effective spark location may shift within and/or along the exposed metal region 829 while maintaining a controlled or substantially controlled consistent spark gap, such as described with reference to FIGS. 6A and 7.

In the depicted example, the exposed conductor region 829 is configured such that exposed conductive surfaces remain within a radial envelope defined by an outer profile of the PSSE module. Maintaining exposed conductor and emitter body surfaces within this radial envelope may, for example, contribute to compact radial packaging of the PSSE module, as described with reference to FIGS. 3-6B.

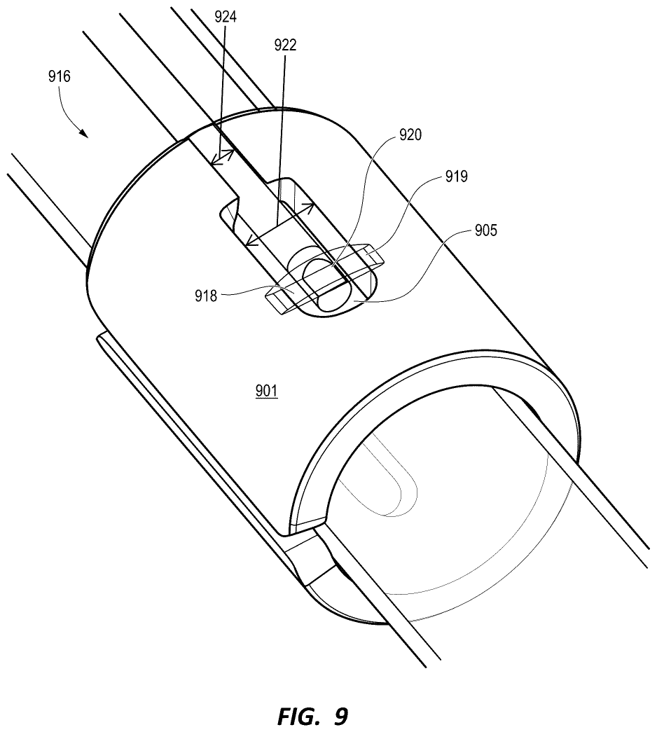

FIG. 9 depicts an enlarged perspective view of an example progressive spark surface emitter (PSSE) module illustrating a localized spark-generating region formed through a capsule and emitter body. The view may illustrate relative positioning of a capsule 901, an exposed discharge aperture 905, and portions of a conductor and emitter body that may participate in spark generation.

In the illustrated embodiment, the PSSE module may include a capsule 901 that may surround at least a portion of an emitter body and one or more conductors. The capsule 901 may be formed of a dielectric and/or otherwise electrically insulative material. The capsule 901 may encapsulate conductive components while allowing selected regions to be exposed. A discharge aperture 905 may be formed through the capsule 901 to provide access to a localized spark-generating region.

One or more conductors may extend longitudinally through and/or adjacent to the PSSE module. In the illustrated example, a portion of a conductor may be exposed within the discharge aperture 905 by way of a cut, skive, or material-removal feature 919 formed through conductor insulation and/or the capsule 901. The cut or skive 919 may expose a conductor surface 920 at a desired longitudinal and circumferential position relative to an exposed emitter body surface.

An exposed emitter body surface 918 may be positioned within the discharge aperture 905 opposite the exposed conductor surface 920. The exposed conductor surface 920 and the exposed emitter body surface 918 may be separated by one or more spark gaps. During application of a high-voltage pulse, electrical charge may concentrate across the spark gap, which may lead to ionization of an intervening fluid medium and formation of an electrical discharge.

In some embodiments, the geometry of the discharge aperture 905, the exposed conductor surface 920, and the exposed emitter body surface 918 may be selected to influence electric-field concentration and spark initiation behavior. The cut or skive 919 may have a tapered, angled, or contoured profile that may control the exposed surface area of the conductor and promote predictable spark formation. As repeated discharge events occur, material at the exposed conductor surface 920 and/or the exposed emitter body surface 918 may be progressively eroded.

In some embodiments, the configuration illustrated in FIG. 9 may allow the effective spark location to migrate along the exposed emitter body surface 918 as erosion progresses, while maintaining a substantially controlled consistent spark gap. Such progressive behavior may distribute erosion over a larger surface area and may contribute to consistency of pressure-wave generation over multiple emission cycles.

The discharge aperture 905 and exposed conductive surfaces may be positioned such that they remain within a radial envelope defined by an outer profile of the capsule 901. Maintaining the spark-generating region within this radial envelope may contribute to compact radial packaging of the PSSE module. Such compact packaging may be advantageous for intravascular lithotripsy catheter systems that include additional internal features, such as guidewire lumens configured to accommodate relatively large guidewires.

Accordingly, FIG. 9 may illustrate an example configuration in which selective removal of insulating material via a cut or skive 919 and formation of a discharge aperture 905 through the capsule 901 may cooperate to define a controlled spark-generating region that supports progressive erosion behavior, consistent spark formation, and compact emitter module geometry.

The PSSE module illustrated in FIG. 9 may be an example implementation of the emitter module 116 described with reference to FIG. 1 and/or the PSSE module 316 described with reference to FIG. 3, configured with another example emitter body geometry relative to the embodiments described with reference to FIGS. 3-8.

As shown in FIG. 9, the PSSE module includes an emitter body disposed around an inner shaft. The inner shaft 916 may be an example implementation of the inner shaft 114 described with reference to FIG. 1 and/or the inner shaft 414 described with reference to FIG. 4. The emitter body may be an example implementation of the emitter body 403 described with reference to FIG. 4 and/or the ring electrode member 1906 described with reference to FIGS. 19A-19D. One or more conductors are positioned relative to the emitter body to define one or more spark-generating regions, such as described with reference to FIGS. 6A-7.

As shown in FIG. 9, the emitter body includes one or more discharge apertures formed through a wall of the emitter body. The discharge apertures may be example implementations of the discharge aperture 405 described with reference to FIG. 4 and/or the discharge aperture 705 described with reference to FIG. 7. One or more exposed conductor regions are defined at locations along the emitter body where encapsulating material is thinned, removed, and/or otherwise modified to expose conductive surfaces of the conductor and the emitter body. The exposed conductor regions may be example implementations of the exposed metal regions 629 described with reference to FIG. 6A and/or the exposed conductor region 829 described with reference to FIGS. 8A-8B.

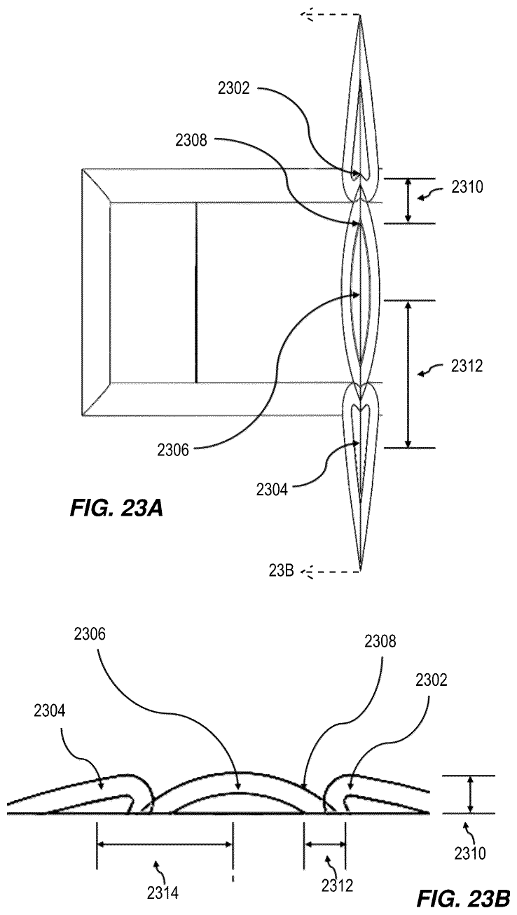

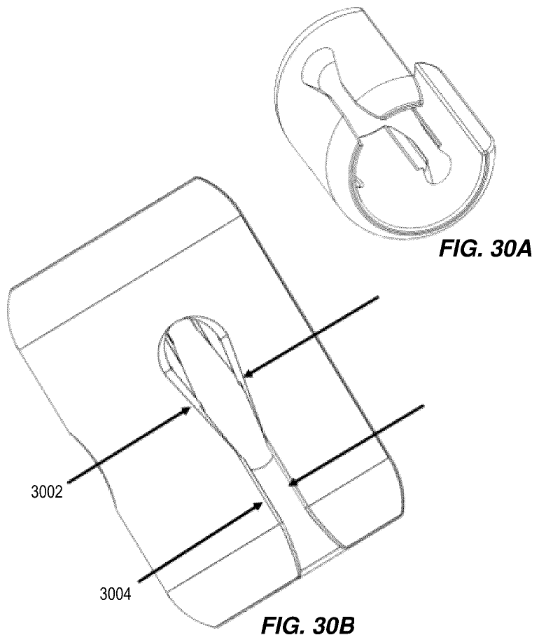

The emitter body geometry illustrated in FIG. 9 may differ from the embodiments of FIGS. 3-8 in one or more geometric parameters, including emitter body wall thickness, discharge aperture shape, discharge aperture depth, and/or conductor channel geometry. Such geometric differences may, for example, affect spark gap characteristics including breakdown voltage, discharge current magnitude, spark initiation location, and/or erosion path behavior, as described with reference to FIGS. 23A-23B and FIGS. 30A-30B.

As further shown in FIG. 9, a larger channel width 922 and a narrower channel width 924 are defined at an outermost surface of the emitter body. The larger channel width 922 corresponds to a wider region of the conductor channel at the outermost surface, such as at and/or near an initial spark generation region, such as the initial spark generation region 1914 described with reference to FIGS. 19A-19D. The narrower channel width 924 corresponds to a retention portion of the conductor channel at the outermost surface, in which the channel width at the outermost surface is reduced relative to the larger channel width 922. In the retention portion defined by the narrower channel width 924, the channel width at the outermost surface of the emitter body is less than a diameter of a discharge conductor disposed within the conductor channel, such that the discharge conductor is mechanically retained within the conductor channel, such as without adhesive bonding and/or additional external support members, consistent with the conductor retention geometry described with reference to the conductor retaining surfaces 515 of FIG. 5, the shaped internal surface of the wire electrode channel 2702 described with reference to FIGS. 27A-27C.

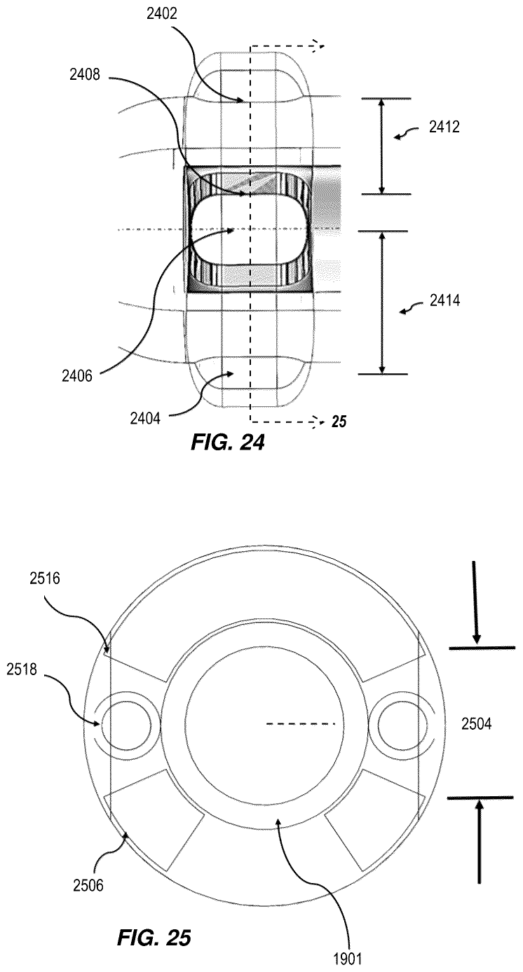

The larger channel width 922 and the narrower channel width 924 cooperate to define both a spark-generating region and a conductor retention region within a single conductor channel geometry. The larger channel width 922 at the initial spark generation region may, for example, define a wider discharge aperture that exposes a larger surface area of the conductor and emitter body for spark generation, such as described with reference to FIGS. 24-26. The narrower channel width 924 at the retention portion may, for example, mechanically retain the discharge conductor within the conductor channel while also defining a narrower plasma confinement geometry for subsequent spark generation events as the conductor erodes along the spark progression path, consistent with the channel width variation geometry described with reference to FIGS. 30A-30B. Such variation in channel width along the spark progression path may, for example, cause discharge current magnitude to vary predictably as spark initiation locations migrate from the larger channel width 922 region toward the narrower channel width 924 region, consistent with the voltage-current decoupling architecture described with reference the mathematical relationship I∝W_{channel} described with reference to FIGS. 30A-30B.

FIG. 10 depicts a perspective view of an example progressive spark surface emitter (PSSE) module 1016 configured in an example windowless arrangement. The PSSE module 1016 may be an example implementation of the emitter module 116 described with reference to FIG. 1 and/or the PSSE module 316 described with reference to FIG. 3. In the illustrated embodiment, the PSSE module 1016 is disposed around an inner shaft 1014. The inner shaft 1014 may be an example implementation of the inner shaft 114 described with reference to FIG. 1 and/or the inner shaft 414 described with reference to FIG. 4.

The PSSE module 1016 includes a capsule 1001 surrounding at least a portion of an emitter body and one or more conductors 1012. The capsule 1001 may be an example implementation of the capsule 301 described with reference to FIG. 3 and/or the capsule 701 described with reference to FIG. 7. The capsule 1001 is formed of a dielectric and/or otherwise electrically insulative material and encapsulates conductive components while allowing selected regions to be exposed for spark generation. In the windowless configuration illustrated in FIG. 10, a through-aperture is not formed through the emitter body itself. Instead, one or more exposed conductor regions 1029 are formed by selective shaping, thinning, and/or removal of material from the capsule 1001.

The exposed conductor region 1029 exposes a conductor surface 1020 and an emitter body surface 1018 at adjacent locations through the capsule 1001. The exposed conductor surface 1020 and the exposed emitter body surface 1018 are separated by a spark gap 1011 defined by the geometry of the capsule-formed cavities rather than by a discharge aperture through the emitter body. The conductor surface 1020 and emitter body surface 1018 may be example implementations of the conductor surface 120 and emitter body surface 118 described with reference to FIG. 1.

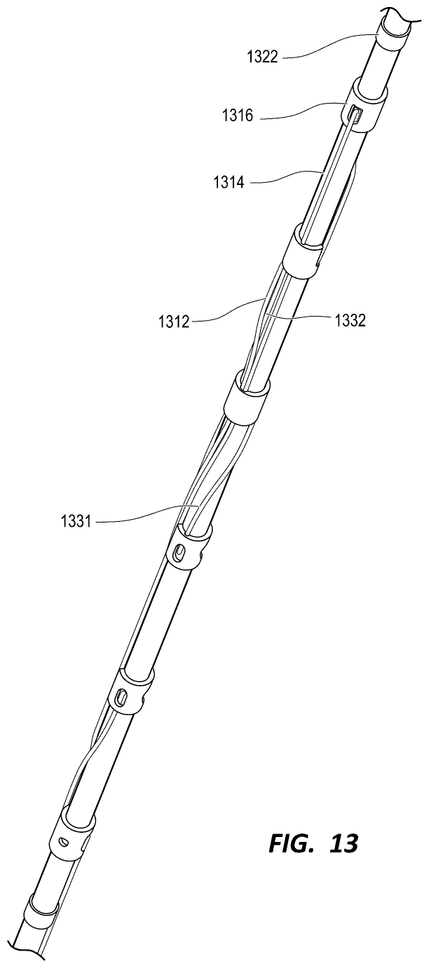

A windowless configuration, such as illustrated in FIG. 10, may, for example, advantageously enable a narrower effective aperture for spark formation compared to embodiments in which a discharge aperture is formed through the emitter body, such as described with reference to FIGS. 3-9. For example, when multiple conductors 1012 are routed longitudinally along the inner shaft 1014—such as conductors supplying power to emitter modules located distally along the catheter—the available radial space may be constrained. Windowless embodiments may, for example, allow these conductors 1012 to pass longitudinally through the emitter body region, such as without a discharge aperture wider than a corresponding conductor channel. By avoiding a through-window in the emitter body, the windowless configuration may, for example, allow additional conductors to be routed longitudinally while maintaining a target maximum cross-sectional width of the PSSE module 1016. Such configurations may, for example, allow an increased number of conductors to be accommodated—for example, increasing from six conductors to eight conductors or more—without increasing the overall radial profile of the emitter module, such as further described with reference to FIGS. 12 and 13.

In some embodiments, the geometry and surface area of the exposed emitter body surface 1018 and the exposed conductor surface 1020 may be selected to influence electric-field concentration and spark initiation behavior. Without being bound to a particular theory, relatively small exposed surface areas may, for example, reduce the time to build sufficient charge concentration across the spark gap 1011 to initiate a gaseous discharge, which may, for example, reduce spark rise time and/or increase consistency of spark formation. As repeated discharge events occur, material at the exposed conductor surface 1020 and/or the exposed emitter body surface 1018 may be progressively eroded. Such erosion may, for example, allow an effective spark location to migrate along the exposed emitter body surface 1018 while maintaining a controlled or substantially controlled consistent spark gap 1011, as described with reference to FIGS. 3 and 6A.

By way of example and not limitation, the exposed conductor region 1020 may migrate along the path defined by the underlying conductor 1012 as material is eroded during operation. The exposed emitter body region 1018 may, for example, shift along the edge of the channel. In some embodiments, for example, as dielectric material is eroded away along with conductor material, the separation between these regions may be maintained by blank space left as conductive and dielectric material is worn away.

In some embodiments, the windowless PSSE module 1016 may further include one or more conductive inserts disposed between the emitter body and the capsule 1001. A conductive insert may be positioned to provide a conductive path between the emitter body surface 1018 and an outer surface of the capsule 1001 and/or between the conductor surface 1020 and the outer surface of the capsule 1001. Insert-based embodiments may, for example, facilitate assembly of PSSE modules by allowing conductive elements to be placed rapidly and accurately prior to encapsulation. In some embodiments, conductive inserts may be formed of materials selected for higher electrical conductivity to focus spark formation on a desired surface, for increased durability to reduce erosion rate during repeated emission cycles, and/or configured to be replaceable or interchangeable to tailor emitter performance.

FIG. 11 depicts a perspective view of an example progressive spark surface emitter (PSSE) module pre-encapsulation assembly 1107 illustrating a curvilinear (e.g., helical) erosion-guiding configuration. The pre-encapsulation assembly 1107 may be an example implementation of the pre-capsulation configurations described with reference to FIGS. 3 and 4, configured with a non-linear conductor arrangement rather than a linear one. The pre-encapsulation assembly 1107 is disposed around an inner shaft of an intravascular lithotripsy catheter.

In the illustrated embodiment, one or more conductors are arranged such that exposed conductive regions follow a non-linear path along the pre-encapsulation assembly 1107. The non-linear path may, for example, be curvilinear relative to the longitudinal axis of the inner shaft. A helical path 1131 of a slot and/or window may, for example, guide a direction of erosion along a target curvilinear path. The slot may be a channel and/or a discharge aperture, such as a helical version of the apertures described with reference to FIGS. 3-5. One or more conductors 1132 may, for example, be arranged in a pattern matching the helical path 1131 in the emitter body and/or around the inner shaft, partially or completely. Multiple spark events may cause erosion of the conductor 1132 along the helical path 1131.

A curvilinear conductor arrangement, such as illustrated in FIG. 11, may, for example, distribute spark initiation locations and electrode erosion along a non-linear path rather than concentrating erosion at a single point and/or along a single linear axis. Such distribution may, for example, advantageously extend emitter operational life and/or distribute pressure wave generation around a larger portion of the emitter circumference relative to linear configurations described with reference to FIGS. 3-10. The non-linear erosion path may also, for example, provide a broader range of achievable spark gap geometries for controlling pressure wave characteristics over the operational life of the emitter. The helical configuration may be an example implementation of the non-linear conductor channel.

An example configuration may, for example, have a shaft 1133 (which may, for example, correspond to inner shaft 114, inner shaft 414, and/or inner shaft 614) in which one or more conductor sets (e.g., wire conductors such as disclosed herein) follow a non-linear path (e.g., corresponding to non-linear path 1131).

In some embodiments, the conductors 1135 may include one or more inflection regions along the conductor channel. The inflection regions may define locations along the non-linear path at which the direction of curvature changes, such as from a first circumferential direction to an opposing circumferential direction. Such inflection geometry may, for example, distribute spark initiation locations across a wider circumferential arc relative to configurations having a single continuous curvature, and/or may, for example, provide additional design flexibility in controlling the spatial distribution of pressure wave generation.

As disclosed with respect to FIG. 11, non-linear conductor and/or channel configurations may take a variety of geometric forms, including helical, curvilinear, inflected, and/or width-varying paths. FIG. 11 depicts, for example, a curvilinear path of a conductor 1135 relative to a linear, longitudinally parallel path such as shown in other figures. The various forms may share a common feature that spark initiation locations migrate along a non-linear predetermined spark progression path, such as electrode material erodes during repeated discharge events. Such configurations may, for example, distribute erosion over a larger electrode surface area, extend emitter operational life, and/or distribute pressure wave generation around a larger portion of the emitter circumference, such as compared to linear configurations described with reference to FIGS. 3-10.

FIG. 12 depicts a cross-sectional view of an example intravascular lithotripsy (IVL) catheter assembly, illustrating an axial cross-section of a catheter 1204, balloon 1206, inner shaft 1214, and conductive wires 1212 arranged within the catheter assembly. The catheter assembly illustrated in FIG. 12 may be an example implementation of the IVL assembly 100 described with reference to FIG. 1.

As shown, the catheter 1204 defines an outer boundary of the assembly. The balloon 1206 is disposed within and/or around a portion of the catheter 1204. The inner shaft 1214 extends longitudinally within the balloon 1206. The inner shaft 1214 defines or surrounds a guidewire lumen. The inner shaft 1214 may be an example implementation of the inner shaft 114 described with reference to FIG. 1. In some embodiments, the guidewire lumen defined by the inner shaft 1214 may be sized to accommodate relatively large guidewires, including guidewires having diameters of between 0.025-inch (0.635 mm) to 0.095-inch (2.413 mm), or between about 0.025 inch (0.635 mm) and about 0.095 inch (2.413 mm), approximately or equal to 0.035 inch (0.889 mm) or larger, as described with reference to FIGS. 1-5.

Conductive wires 1212 are arranged within the catheter assembly, distributed around the inner shaft 1214. The conductive wires 1212 may be example implementations of the conductors 112 described with reference to FIG. 1. As shown, the conductive wires 1212 are positioned outside the guidewire lumen and within the radial space between the inner shaft 1214 and the catheter 1204. In the illustrated embodiment, six conductive wires 1212 are shown arranged in a circumferential pattern around the inner shaft 1214. Such circumferential arrangement may, for example, allow the target conductor count to be accommodated without occupying the guidewire lumen. Such arrangement may, for example, advantageously facilitate compatibility with larger guidewires, including guidewires between 0.025-inch (0.635 mm) to 0.095-inch (2.413 mm), or between about 0.025 inch (0.635 mm) and about 0.095 inch (2.413 mm), such as 0.035-inch guidewires (0.889 mm), while maintaining a compact overall catheter profile.

In some embodiments, the number of conductive wires 1212 may differ from the illustrated example and may be increased or decreased depending on the number of emitter modules, emitter pairs, and/or electrical configurations used. For example, some embodiments may include eight or more conductive wires, such as accommodating additional emitter modules (e.g., as further described with reference to FIG. 13). The ability to route additional conductors within the radial envelope of the catheter 1204 without increasing the outer catheter diameter may, for example, advantageously facilitate extended treatment lengths and/or increased emitter module counts suitable for peripheral vessel applications.

The axial configuration illustrated in FIG. 12 may, for example, allow multiple emitter modules supplied by the conductive wires 1212 to be distributed longitudinally within the balloon 1206, enabling delivery of pressure waves along an extended axial length suitable for larger peripheral vessels. The combination of larger balloon dimensions, a guidewire lumen sized for larger guidewires, and/or the illustrated conductor arrangement may, for example, be particularly advantageous for peripheral IVL procedures that benefit from enhanced stability, support, and/or energy distribution.

FIG. 13 depicts a perspective view of a distal portion of an intravascular lithotripsy catheter illustrating an example longitudinal arrangement of multiple progressive spark surface emitter (PSSE) modules and associated electrical components along an inner shaft 1314. The illustrated view represents a treatment region configured for use in larger vessels and/or longer lesion segments. The configuration illustrated in FIG. 13 may be an example implementation of the IVL assembly 100 described with reference to FIG. 1, shown with multiple emitter modules 1316 arranged along an extended treatment length.

Multiple emitter modules 1316 are disposed along the inner shaft 1314. The emitter modules 1316 may be example implementations of the emitter module 116 described with reference to FIG. 1 and/or the PSSE module 316 described with reference to FIG. 3. The emitter modules 1316 are longitudinally spaced along the inner shaft 1314 within a treatment region of the catheter. Such longitudinal spacing may, for example, allow delivery of lithotripsy pressure waves along an extended axial length. Extended axial length of treatment may, for example, be particularly advantageous when treating longer calcified segments in peripheral vessels.

One or more conductive wires 1312 extend longitudinally along the inner shaft 1314. The one or more conductive wires 1312 are electrically coupled to respective emitter modules 1316. The conductive wires 1312 may be example implementations of the conductors 112 described with reference to FIG. 1 and/or the conductive wires 1212 described with reference to FIG. 12. As shown, the conductive wires 1312 are routed along the inner shaft 1314 within the radial envelope of the catheter, such that conductor routing does not occupy the guidewire lumen. Such routing may, for example, advantageously preserve radial space for a guidewire lumen sized to accommodate relatively large guidewires, including guidewires having diameters of between 0.025-inch (0.635 mm) to 0.095-inch (2.413 mm), or between about 0.025 inch (0.635 mm) and about 0.095 inch (2.413 mm), approximately or equal to 0.035 inch (0.889 mm), or larger.

One or more bridge conductors 1331 are shown connecting adjacent emitter modules 1316 along the inner shaft 1314. The bridge conductors 1331 may, for example, electrically couple emitter modules 1316 to one another and/or to the conductive wires 1312. The bridge conductors may, for example, distribute electrical energy from a proximal control module to distally located emitter modules, such as without a dedicated proximally routed conductor for each emitter module. Such bridge conductor arrangements may, for example, reduce the total number of long, proximally routed conductors within the catheter. Such reduction may, for example, advantageously preserve radial space, such as for a larger guidewire lumen, while still enabling delivery of lithotripsy energy across an extended treatment length.

A return conductor 1332 extends longitudinally along the inner shaft 1314. The return conductor 1332 provides a return current path to close the discharge circuit for each spark event. The return conductor 1332 may be an example implementation of the return conductor 232 described with reference to FIG. 2. As shown, the return conductor 1332 is routed along the inner shaft 1314 within the radial envelope of the catheter alongside the conductive wires 1312. In some embodiments, a single return conductor 1332 may be used. In some embodiments, less return conductors 1332 may be used than supply conductors. In some embodiments, an equal or greater number of return conductors 1332 may be used than supply conductors.

One or more markers 1322 are disposed along the catheter at or near locations corresponding to the emitter modules 1316 and/or ends of the treatment region. The markers 1322 may be example implementations of the markers 122 described with reference to FIG. 1. The markers 1322 are formed of a radiopaque and/or otherwise imageable material and may, for example, facilitate visualization of the catheter and emitter array under fluoroscopy and/or other imaging modalities, assisting an operator in positioning the emitter modules 1316 relative to a target lesion.

In some embodiments, such as shown, the longitudinal configuration illustrated in FIG. 13 may, for example, allow multiple emitter modules 1316, conductive wires 1312, bridge conductors 1331, and the return conductor 1332 to be accommodated along an extended treatment length without increasing the outer diameter of the catheter. Routing the conductive elements along the inner shaft 1314 and within the catheter's radial envelope may, for example, advantageously enable delivery of lithotripsy energy over a longer segment while preserving compatibility with larger guidewire platforms, such as further described with reference to FIGS. 1 and 12.

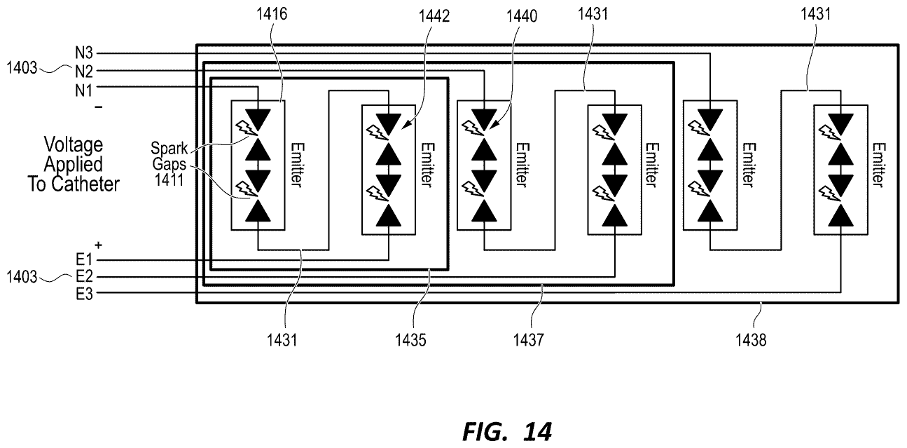

FIG. 14 depicts an example electrical schematic illustrating a catheter-based intravascular lithotripsy system including multiple progressive spark surface emitter (PSSE) modules electrically coupled to a control module 1403. The schematic represents an example configuration of electrical interconnections between the control module 1403 and multiple emitter modules arranged along a catheter. The electrical architecture illustrated in FIG. 14 may be an example implementation of the electrical configuration described with reference to the IVL assembly 100 of FIG. 1 and the emitter modules 1316 described with reference to FIG. 13.

The control module 1403 is electrically coupled to one or more conductor pairs extending to the catheter. The control module 1403 may, for example, be configured to selectively apply electrical energy, such as high-voltage pulses, to the catheter through one or more conductors. When energized, the applied voltage may be delivered to one or more emitter modules to generate electrical discharge across corresponding spark gaps 1411. The spark gaps 1411 may be example implementations of the spark gaps described with reference to the emitter modules 116 of FIG. 1 and the exposed conductor regions 629 of FIG. 6A.

In the illustrated embodiment, the emitter modules are arranged in multiple selectively energizable configurations E1, E2, and E3. Each configuration E1, E2, and E3 includes one or more emitter modules electrically coupled to the control module 1403 through respective conductor sets N1, N2, and N3. The conductor sets N1, N2, and N3 may be example implementations of the conductive wires 1312 described with reference to FIG. 13. In a first configuration E1, a first group of emitter modules is energized by a first set of conductors N1. In a second configuration E2, a second group of emitter modules is energized by a second set of conductors N2. In a third configuration E3, a third group of emitter modules is energized by a third set of conductors N3. The third configuration E3 may, for example, be deployed in larger vessels and/or longer calcifications, such as in peripheral IVL applications of the type described with reference to FIGS. 1 and 15.

One or more bridge conductors 1431 electrically couple adjacent emitter modules and/or groups of emitter modules within the schematic. The bridge conductors 1431 may be example implementations of the bridge conductors 1331 described with reference to FIG. 13. The bridge conductors 1431 provide an electrical connection between emitter modules such that voltage applied to one emitter module may be transferred to one or more additional emitter modules. In some embodiments, the bridge conductors 1431 may function as floating conductors that enable sequential and/or daisy-chained activation of multiple emitter modules during a therapy cycle. In some embodiments, the bridge conductors 1431 may differ in size, length, and/or electrical characteristics relative to conductors that connect directly to the control module 1403. For example, bridge conductors 1431 may be shorter than conductors extending to the control module 1403, which may, for example, reduce electrical resistance and/or increase power transfer efficiency.

The schematic further illustrates selectively energizable configurations 1435, 1437, and 1438. The selectively energizable configurations 1435, 1437, and 1438 represent different groupings and/or subsets of emitter modules that may be independently or collectively activated by the control module 1403. Such configurations may, for example, allow the number and/or distribution of active emitter modules to be adjusted based on treatment length, vessel size, and/or clinical considerations. In some embodiments, selectively energizable configurations may allow a subset of emitter modules to be activated for shorter lesion segments, while additional configurations may be activated for longer and/or more complex lesion morphologies, such as those encountered in peripheral vessels.

The emitter module 1416 is shown coupled within the schematic and may be an example implementation of the emitter module 116 described with reference to FIG. 1 and/or the emitter modules 1316 described with reference to FIG. 13. The emitter module 1442 is also shown within the schematic and may represent an additional emitter module or group coupled within one of the selectively energizable configurations. The emitter module 1440 is shown coupled to the bridge conductor 1431 and may represent an emitter module activated via bridge coupling rather than by a direct conductor from the control module 1403.

The multi-configuration electrical architecture illustrated in FIG. 14 may, for example, be particularly advantageous for catheter systems configured to accommodate relatively large guidewires, such as guidewires having diameters of between 0.025-inch (0.635 mm) to 0.095-inch (2.413 mm), or between about 0.025 inch (0.635 mm) to about 0.095 inch (2.413 mm), approximately or equal to 0.035 inch (0.889 mm), or even larger, as described with reference to FIGS. 1 and 15. Catheter designs intended for use with larger guidewire lumens may have increased demands on internal space for guidewire support, structural reinforcement, and conductor routing. By using bridge conductors 1431 and selectively energizable configurations 1435, 1437, and 1438, the illustrated architecture may, for example, reduce the total number of long, proximally routed conductors within the catheter. This may, for example, help preserve radial space for a larger guidewire lumen while still enabling delivery of lithotripsy energy across an extended treatment length. Such arrangements may, for example, be particularly suitable for peripheral and large-vessel applications where enhanced guidewire support and longer treatment zones are desirable.

FIG. 15 depicts an example intravascular lithotripsy (IVL) assembly illustrating a proximal-to-distal arrangement of catheter components as they may be used during a peripheral vascular treatment procedure. The IVL assembly illustrated in FIG. 15 may be an example implementation of the IVL assembly 100 described with reference to FIG. 1, shown in the context of a catheter system, such as (by way of example and not limitation) configured for peripheral use.

The IVL assembly includes a proximal interaction module 1502 from which a catheter extends distally. The proximal interaction module 1502 may be an example implementation of the proximal interaction module 102 described with reference to FIG. 1. The catheter includes an outer shaft extrusion 1546. The outer shaft extrusion may, for example, define a structural body of the catheter, and a guidewire lumen extrusion 1548 extending longitudinally through at least a portion of the catheter. The outer shaft extrusion 1546 and guidewire lumen extrusion 1548 may be example implementations of the catheter 104 and inner shaft 114 described with reference to FIG. 1. During use, a guidewire (GW) extends through the guidewire lumen extrusion 1548. The GW may, for example, provide a rail for advancing and positioning the catheter. In some embodiments, the guidewire lumen extrusion 1548 may be sized to accommodate relatively large guidewires, such as guidewires having diameters of between 0.025-inch (0.635 mm) to 0.095-inch (2.413 mm), or between about 0.025 inch (0.635 mm) and about 0.095 inch (2.413 mm), approximately or equal to 0.035 inch (0.889 mm), or larger, which may, for example, provide enhanced support and stability for peripheral and/or large-bore vascular navigation, as described with reference to FIGS. 1 and 12.

A working length 1544 of the IVL assembly extends distally from the proximal interaction module 1502. The working length 1544 includes a treatment region of the catheter that is advanced into the blood vessel and positioned adjacent to a target lesion. In some embodiments, the working length 1544 may include an inflatable balloon and one or more emitter modules disposed along the catheter, such as described with reference to FIGS. 1 and 13, allowing lithotripsy energy to be delivered over an extended axial region suitable for longer lesion segments and/or larger vessels. In some examples, a working length 1544 of 1000 mm or greater may be used. In some examples, a working length of 1100 mm or greater may be used. In some examples, a working length of 1200 mm or greater may be used. In some examples, a working length of 1300 mm or greater may be used. In some examples, a working length of 1400 mm or greater may be used.

Electrical energy for operating the emitter modules along the working length 1544 is supplied through one or more conductors extending from an external energy source. In the illustrated example, electrical connectivity is provided via an electrical cable 1554 extending from the proximal interaction module 1502. The electrical cable 1554 terminates in an electrical connector 1556 configured to couple, either releasably or fixedly, to a control module. During use, the electrical cable 1554 and connector 1556 allow high-voltage pulses and/or control signals to be delivered from the control module to the emitter modules disposed along the catheter, such as described with reference to FIG. 14.

The proximal interaction module 1502 further includes a guidewire interface port 1550 that provides access to the guidewire lumen extrusion 1548. During use, a guidewire may be introduced, advanced, exchanged, and/or withdrawn through the guidewire interface port 1550 while the catheter remains positioned within the vessel. The guidewire interface port 1550 may be configured to maintain fluid sealing during operation, which may, for example, be particularly advantageous during over-the-wire procedures using larger guidewire platforms, such as guidewires between 0.025-inch (0.635 mm) to 0.095-inch (2.413 mm), or between about 0.025 inch (0.635 mm) and about 0.095 inch (2.413 mm), including 0.035-inch (0.889 mm) guidewires, as described with reference to FIGS. 1 and 12.

The proximal interaction module 1502 also includes a balloon interface port 1552 configured to allow inflation and/or deflation of a balloon disposed along the working length 1544. The balloon interface port 1552 may be an example implementation of the fluidic interface described with reference to the proximal interaction module 102 of FIG. 1. During use, a fluid source such as a syringe and/or pump may be coupled to the balloon interface port 1552, such as to inflate the balloon (e.g., for vessel wall apposition during lithotripsy) and/or to deflate the balloon (e.g., for repositioning and/or withdrawal of the catheter).

One or more conductors 1512 extend longitudinally along the catheter within the working length 1544. The conductors 1512 may be an example implementation of the conductors 112 described with reference to FIG. 1 and the conductive wires 1312 described with reference to FIG. 13. The conductors 1512 are routed outside the guidewire lumen extrusion 1548, such as along peripheral regions of the outer shaft extrusion 1546, such that conductor routing does not occupy the guidewire lumen. Such routing may, for example, preserve radial space for the guidewire lumen extrusion 1548 while accommodating the conductor count associated with multiple emitter modules along the working length 1544, as described with reference to FIGS. 12 and 13.

In this example, the conductors 1512 are schematically shown communicably coupled to a power source 1560 (e.g., a voltage source such as a pulse generator). The power source 1560 is schematically shown as communicably coupled to a control module 1562. The control module 1562 may be operated (e.g., automatically, manually) to operate the power source 1560. The power source 1560 may, for example, provide power (e.g., pulse), such as to generate a spark(s) at one or more emitter modules. In some embodiments, the control module 1562 may be integrated in the power source 1560.

Accordingly, FIG. 15 illustrates an IVL catheter assembly architecture in which the outer shaft extrusion 1546, guidewire lumen extrusion 1548, working length 1544, proximal interaction module 1502, and associated electrical and fluid interfaces cooperate during use, such as advantageously enabling advancement of the catheter over a guidewire, stable positioning within peripheral and/or large-bore vessels, and/or controlled delivery of intravascular lithotripsy therapy, such as even over extended treatment lengths.

FIG. 16 depicts an example emission cycle plot 1600 illustrating various parameters over time during operation of a progressive spark surface emitter (PSSE) module, such as the emitter modules 116 described with reference to FIG. 1 and/or the PSSE module 316 described with reference to FIG. 3.

A source voltage overtime 1602 is depicted as a dashed line. The source voltage overtime 1602 represents the variation of voltage applied from the power source, such as the control module 1403 described with reference to FIG. 14. The source voltage may rise during a ramp-up phase prior to reaching a dwell period and subsequently ramping down. The duration of the dwell period may, for example, depend on a time to form an ionized gaseous bridge across a spark gap, such as the spark gaps 1411 described with reference to FIG. 14. In some embodiments, dwell times may be approximately or equal to 15 to 50 milliseconds. Example tests have found that a dwell time of approximately or equal to 20-30 milliseconds, such as approximately or equal to 25 milliseconds, may provide sufficient time to consistently generate a spark in some embodiments without overheating the emitter body.

An emitter voltage overtime 1604 is represented by a solid line. The emitter voltage overtime 1604 illustrates the voltage behavior at the emitter during operation. The emitter voltage may, for example, exhibit a slight lag during the ramp-up phase compared to the source voltage and/or a sharper drop-off. The lag may, for example, result from resistance within the conductor path. The sharp drop-off may, for example, result from spark formation.

A source rise time 1610 corresponds to the rise time of the source voltage overtime 1602 as initially introduced into the system. As this voltage encounters different spark locations, a mechanical design of each circuit element in series may be configured to modify the rise time to a distinct value characteristic of that location and/or group of locations. At a first spark location, the emitter may be configured with a transfer function corresponding to a first rise time 1612. At a second spark location, the emitter may be configured with a second transfer function corresponding to a second rise time 1614. Such variation between the first emitter voltage over time 1604 and a second emitter voltage over time 1608 may, for example, be achieved through differences in mechanical and/or electrical configuration at various spark gaps, such as adjustments to geometry, size, and/or length of the conductors and/or differing material properties. Customized variability in rise times across locations may, for example, enable increased precision in control over timing and delivery of pressure waves.

In some embodiments, the second spark location may have a faster rise time than the first spark location, which may, for example, correspond to a conductive path with lower overall resistance, such as a bridge conductor 1431, shorter path, and/or larger gauge, as described with reference to FIGS. 13 and 14. In some embodiments, the second spark location may have a slower rise time than the first spark location, for example where mechanical parameters are adjusted to delay the rise time of the second spark location relative to the first.

In some embodiments, each emitter module may be defined by a separate transfer function that defines a voltage response and/or spark formation response to a power input. For example, emitter module transfer functions may be customized within a single IVL assembly 100, as described with reference to FIG. 1. A single applied input, such as a high-voltage pulse from the control module 1403, may have a specific rise time when initially introduced into the system. As this voltage encounters different spark locations, the mechanical design of each circuit element in series may modify this rise time to a distinct value characteristic of that location and/or group of locations. This alteration may, for example, be achieved by adjustment and/or selection of localized circuit components such as specific conductor configurations, emitter designs, and/or the use of different dielectric materials.

In some embodiments, bridge conductors, such as the bridge conductors 1431 described with reference to FIGS. 13 and 14, may experience more mechanical damage than other conductors. Without being bound to a particular theory, bridge conductors may have a shorter rise time, as the voltage may be suddenly applied as a spark is formed. Accordingly, the voltage pulse at a bridge conductor may appear more like a steep triangle or square wave than the source voltage over time 1602. Some embodiments may, for example, be configured to approximate the voltage pulse by decreasing rise time of the source voltage and/or shortening wires. Some embodiments may, for example, increase wire gauge of bridge conductors, which may, for example, increase resistance to mechanical damage.

The emitter temperature overtime 1606 is shown as a dotted line. This indicates the thermal profile of the emitter throughout the process. For example, as the emitter voltage peaks and discharges (e.g., in a spark), the emitter temperature may, for instance, follow as the thermal effects propagate through the emitter body. A temperature rise may, for example, be induced by the local excitation of the conductive surfaces as ionized gases are generated.

FIG. 17 depicts an example method 1700 of designing and/or configuring a progressive spark surface emitter (PSSE) module, such as the emitter modules 116 described with reference to FIG. 1 and/or the PSSE module 316 described with reference to FIG. 3. The method 1700 begins at step 1702, at which a first PSSE module is selected for configuration. The method 1700 proceeds through a series of design steps organized into two phases: a first phase directed to overall emitter body configuration, and a second phase directed to aperture-level configuration.

At step 1704, a shape of the emitter body is selected. The shape of the emitter body may, for example, affect the overall profile of the PSSE module and may influence the spatial relationship between the emitter body surface and the conductor surface. For example, the shape may be selected to facilitate compact radial packaging, such as described with reference to FIGS. 5 and 6B, and/or to accommodate a guidewire lumen sized for relatively large guidewires, such as guidewires having diameters of between 0.025-inch (0.635 mm) to 0.095-inch (2.413 mm), or between about 0.025 inch (0.635 mm) and about 0.095 inch (2.413 mm), approximately or equal to 0.035 inch (0.889 mm), or larger.

At step 1710, dimensions of the emitter body are selected. The dimensions may, for example, include outer diameter, inner diameter, wall thickness, and/or longitudinal length of the emitter body. In some embodiments, the wall thickness may be selected to correspond to a conductor diameter, such as described with reference to FIG. 6B, which may, for example, facilitate compact radial packaging and/or manufacturability. The dimensions may also, for example, affect a number of sequential emission cycles that may be expected from the emitter body, as greater wall thickness may provide additional material to accommodate progressive erosion during repeated discharge events.

At step 1706, a conductor size is selected. The conductor size may, for example, be selected based on the dimensions of the emitter body selected at step 1710, such that the conductor diameter corresponds to the emitter body wall thickness. A larger conductor gauge may, for example, enhance energy delivery efficiency, while a smaller conductor gauge may, for example, provide a more compact design, as described with reference to FIG. 6B. The conductor size may also, for example, influence the surface area of the conductor surface exposed within a discharge aperture, which may affect charge concentration behavior and spark initiation characteristics.

At step 1708, a bridge conductor size is selected. The bridge conductor size may, for example, be selected based on the number and/or spacing of emitter modules to be connected, the current-carrying capacity, and/or routing constraints within the catheter. Bridge conductors may, for example, be shorter than conductors extending proximally to a control module, which may, for example, reduce electrical resistance and/or facilitate power transfer efficiency, as described with reference to FIGS. 13 and 14.

At step 1712, a material of the emitter body is selected. The material may, for example, be selected based on electrical conductivity, erosion resistance, biocompatibility, and/or machinability. Material selection may, for example, affect the rate of electrode erosion during repeated discharge events and/or may influence the longevity of the PSSE module over its operational life.

At step 1714, a number of spark-generating apertures is selected. The number of spark-generating apertures may, for example, be selected to achieve a target spark distribution. In some embodiments, for example, uni-directional spark distribution may be achieved by arranging spark-generating apertures along a single path (e.g., curvilinear and/or linear) relative to the longitudinal axis of the catheter. Uni-directional spark distribution may, for example, allow controlled delivery of pressure waves in a single direction, which may, for example, allow a physician to focus pressure waves on a targeted area of calcification. In some embodiments, bi-directional spark distribution may be implemented, for example, by positioning spark-generating apertures along different sides (e.g., opposing sides) of the catheter and/or inner shaft 114. Such configurations may, for example, provide therapeutic pressure waves in two directions. Some embodiments may be configured to provide omni-directional or substantially omni-directional spark distribution, for example by configuring apertures around the circumference of the emitter body (e.g., individual apertures and/or curvilinear apertures), which may, for example, generate pressure waves that act circumferentially around vessel walls and/or reduce rotational positioning demands on the operator.

At decision step 1716, it is assessed whether all apertures of the emitter have been configured. If not, the method proceeds to step 1718. At step 1718, a shape of the aperture is selected. The shape of the aperture may, for example, define an interaction between one or more conductor surfaces 120 and corresponding emitter body surfaces 118. The shape of the aperture may, for example, affect a dimension of the emitter body and/or a number of sequential emission cycles that may be expected.

At step 1720, a size of the aperture is selected. The size of the aperture may, for example, depend on the conductor size selected at step 1706. The size and/or shape of the aperture may, for example, influence the spark gap geometry, which may, for example, affect the voltage to generate a spark, the duration of a power pulse, and/or the ramp-up and/or ramp-down profile of the power pulse. Such factors may, for example, affect the generated spark and corresponding pressure wave magnitude and propagation characteristics.

At step 1722, a number of conductors in the aperture is selected. For example, some apertures may be terminal apertures, some may include a bridge wire, and/or some may include multiple power source conductors. A number and/or placement of conductors may, for example, affect local charge concentration location, available power at a spark gap, and/or thermal effects during operation. For example, more conductors for a given power target may distribute resistance-generated heat, which may, for example, reduce localized heating.

At step 1724, a number of spark gaps is selected. The number of spark gaps may, for example, affect a pulse discharge pattern and/or a longevity of the emitter. For example, additional spark gaps per discharge aperture may, for example, enable sparks to be generated at different surfaces in various emission cycles. A higher number of spark gaps may, for example, enable individual conductive surfaces to be smaller, which may, for example, provide greater flexibility in geometry and/or placement of emitter module components.

At step 1726, a position of the aperture is determined. The position may, for example, depend on the number of apertures selected at step 1714 and/or the desired spark distribution pattern. After the aperture position is determined, the method returns to decision step 1716 to assess whether additional apertures are to be configured. Once all apertures are configured, the method proceeds to decision step 1728.

At decision step 1728, it is determined whether another emitter module is to be configured. If so, the method returns to step 1702 to begin configuration of the next PSSE module. If not, the method ends. In some embodiments, successive PSSE modules configured by the method 1700 may differ in emitter body shape, conductor size, aperture geometry, and/or aperture position, such as to provide different spark gap geometries, pressure wave characteristics, and/or erosion path configurations along a multi-emitter PSSE array, such as described with reference to FIGS. 13 and 14.

FIG. 18 depicts an example method 1800 of operating a progressive spark surface emitter (PSSE)-based intravascular lithotripsy (IVL) assembly, such as the IVL assembly 100 described with reference to FIG. 1.

At step 1802, a catheter with one or more emitter modules is deployed. The catheter may be an example implementation of the catheter 104 described with reference to FIG. 1, including one or more emitter modules such as the emitter modules 116 described with reference to FIG. 1 and/or the PSSE modules 316 described with reference to FIG. 3. Deploying the catheter may, for example, include advancing the catheter over a guidewire through a peripheral and/or coronary vessel to a target treatment site. In some embodiments, the catheter may be advanced over a guidewire having a diameter of between 0.025-inch (0.635 mm) to 0.095-inch (2.413 mm), or between about 0.025 inch (0.635 mm) and about 0.095 inch (2.413 mm), approximately or equal to 0.035 inch (0.889 mm), or larger, such as described with reference to FIGS. 1 and 15, which may, for example, reduce or eliminate guidewire exchange prior to deployment.

At step 1804, the catheter is positioned for treatment. Positioning may, for example, include advancing and/or retracting the catheter along the guidewire until one or more emitter modules are aligned with a target calcified lesion. In some embodiments, step 1804 may include operating one or more balloons, such as the balloon 106 described with reference to FIG. 1. One or more markers, such as the markers 122 described with reference to FIG. 1 and/or the markers 1322 described with reference to FIG. 13, may, for example, facilitate visualization of the catheter and emitter array under fluoroscopy and/or other imaging modalities to assist with positioning.

At step 1806, leads of the catheter are connected to a power source. The leads may be an example implementation of the electrical cable 1554 and connector 1556 described with reference to FIG. 15. Connecting the leads may, for example, include coupling the electrical connector of the proximal interaction module to a control module and/or pulse generator configured to deliver high-voltage pulses to the emitter modules along the catheter, such as the control module 1403 described with reference to FIG. 14. In some embodiments, the leads may be connected to a power source prior to step 1802 and/or step 1804.

At step 1808, a power profile is set. The power profile may, for example, be retrieved as a predetermined power profile, automatically determined, preset, and/or manually configured by a physician and/or technician. The power profile may be selected according to a target therapy, body location, and/or device configuration. The power profile may, for example, include parameters such as applied voltage, pulse duration, pulse repetition rate, and/or total pulse count. In some embodiments, the power profile may include a predetermined voltage schedule corresponding to expected geometric changes along a spark progression path of one or more emitter modules, such as described with reference to the emission cycle plot of FIG. 16. The power profile may, for example, enable configuration of electrical parameters to deliver desired spark characteristics, timing, and/or sequence.

At step 1810, a power pulse is applied. The power pulse application may, for example, be to one or more emitter modules, such as to one or more sets of conductors as described with reference to FIG. 14. Applying the power pulse may, for example, cause one or more emitter modules to generate electrical discharge across corresponding spark gaps, such as the spark gaps 1411 described with reference to FIG. 14, producing pressure waves that propagate through the balloon fluid and into adjacent vessel tissue. The pressure waves may, for example, be part of a therapy such as altering calcification in blood vessels.

At decision step 1812, it is determined whether degassing of the balloon is to be performed. Degassing may, for example, be performed when gas accumulation within the balloon fluid reduces pressure wave propagation efficiency. If degassing is to be performed, the method proceeds to step 1816, at which the balloon is operated to degas. Degassing may, for example, include deflating and/or re-inflating the balloon 106 to expel accumulated gas from the fluid medium. Following step 1816, the method returns to decision step 1814. If degassing is not to be performed, the method proceeds directly to decision step 1814.

At decision step 1814, it is determined whether a target number N of pulses has been applied. If the target number of pulses has not been applied, the method returns to step 1810 to apply an additional power pulse. If the target number of pulses has been applied, the method ends. In some embodiments, the target number N of pulses may correspond to a predetermined total pulse count selected based on expected electrode erosion behavior along the spark progression path of the emitter modules, such as described with reference to FIGS. 3, 6A, and 16. Applying pulses according to a predetermined count may, for example, allow the voltage schedule set at step 1808 to remain synchronized with expected spark gap geometry at each erosion stage, potentially facilitating consistent pressure wave output over the operational life of the emitter modules.

FIG. 19A depicts a perspective view of an example PSSE embodiment. A catheter inner body 1901 extends along a longitudinal centerline 1903. A ring electrode member 1906 is disposed around the catheter inner body 1901. A wire electrode member 1908 is positioned relative to the ring electrode member 1906 to define a spark-generating region. An initial spark generation region 1914 is defined between the ring electrode member 1906 and the wire electrode member 1908. The catheter inner body 1901 may be an example implementation of the inner shaft 114 described with reference to FIG. 1 and/or the inner shaft 414 described with reference to FIG. 4. The ring electrode member 1906 and wire electrode member 1908 may be example implementations of the emitter body 403 and conductor 412 described with reference to FIG. 4.

As shown in FIG. 19A, a cross-section line indicates a transverse plane through the assembly at a longitudinal position corresponding to the initial spark generation region 1914. The cross-sectional views of FIGS. 19B-19D are taken at or near this plane, illustrating the geometric relationships between the ring electrode member 1906, the wire electrode member 1908, and the longitudinal centerline 1903 in the transverse direction.

FIG. 19B depicts a cross-sectional view of the embodiment of FIG. 19A taken at the plane indicated by line 19B. The catheter inner body 1901 is shown in cross-section, surrounded by the ring electrode member 1906. The wire electrode member 1908 is positioned within a channel of the ring electrode member 1906. The initial spark generation region 1914 is defined between opposing surfaces of the ring electrode member 1906 and the wire electrode member 1908 at this cross-sectional plane. A ring electrode channel 1904 is formed in the ring electrode member 1906. The ring electrode channel 1904 receives the wire electrode member 1908. The ring electrode channel 1904 may be an example implementation of the conductor channel 421 described with reference to FIG. 4.

As shown in FIG. 19B, the wire electrode member 1908 is positioned within the ring electrode channel 1904 such that the initial spark generation region 1914 is defined at a location where adjacent opposing surfaces of the ring electrode member 1906 and the wire electrode member 1908 are in closest proximity. The longitudinal centerline 1903 passes through the catheter inner body 1901 at the center of the cross-section.

FIG. 19C depicts a cross-sectional view of the embodiment of FIG. 19A, further illustrating the insulation configuration and spark initiation locations of the ring electrode member 1906 and the wire electrode member 1908. In addition to the components shown in FIG. 19B, FIG. 19C shows a ring electrode insulative member 1910 disposed adjacent to the ring electrode member 1906, and a wire electrode insulative member 1912 disposed adjacent to the wire electrode member 1908. The ring electrode insulative member 1910 and the wire electrode insulative member 1912 may be example implementations of the encapsulating dielectric material described with reference to the capsule 301 of FIG. 3 and/or the capsule 701 of FIG. 7.

The ring electrode insulative member 1910 terminates, defining a ring electrode spark initiation location 1916. The wire electrode insulative member 1912 terminates, defining a wire electrode spark initiation location 1918. The ring electrode spark initiation location 1916 and the wire electrode spark initiation location 1918 are exposed electrode regions bounded by the respective insulative members 1910 and 1912. The insulative material electrically isolates the spark initiation locations from adjacent electrode body portions. The initial spark generation region 1914 is defined between the ring electrode spark initiation location 1916 and the wire electrode spark initiation location 1918.

The ring electrode channel 1904 is shown in FIG. 19C as a narrow channel formed in the ring electrode member 1906. The ring electrode channel 1904 may, for example, constrain plasma expansion during electrical discharge, such as described with reference to the voltage-current decoupling geometry described herein. The ring electrode insulative member 1910 and wire electrode insulative member 1912 bound the spark initiation locations 1916 and 1918 such that electrical discharge between the ring electrode member 1906 and the wire electrode member 1908 is physically inhibited except at the initial spark generation region 1914, such as described with reference to the insulation-defined discharge region described herein.

Also shown in FIG. 19C are an outer diameter of the ring electrode member 1928 and an inner diameter of the ring electrode member 1930, defining an example annular geometry of the emitter module. A diameter of the wire electrode member 1926 is also shown, illustrating the relative sizing of the wire electrode member 1908 relative to the wall thickness of the ring electrode member 1906. In some embodiments, the diameter of the wire electrode member 1926 may correspond to the wall thickness of the ring electrode member 1906, such as described with reference to FIG. 6B. For example, the diameter of the wire electrode member may be at least 80% of the thickness of the ring electrode member. The diameter may, for example, be at least 85% of the thickness. The diameter may, for example, be at least 90% of the thickness. The diameter may, for example, be at least 95% thickness. A relationship closer to 1:1 may, for example, advantageously enable a thinner radial profile. Various embodiments may, for example, facilitate compact radial packaging and/or manufacturability.

An inner radial distance 1920 extends from the longitudinal centerline 1903 to an inner boundary of the ring electrode member 1906. An outer radial distance 1922 extends from the longitudinal centerline 1903 to an outer boundary of the ring electrode member 1906. A common radial envelope 1924 encompasses both the ring electrode member 1906 and the wire electrode member 1908, defined between the inner radial distance 1920 and the outer radial distance 1922. As shown, the emitter body (e.g., ring electrode member 1906) and the conductor (e.g., wire electrode member 1908) may, for example, advantageously be contained within the common radial envelope 1924.

FIG. 19D depicts a further cross-sectional view of the embodiment of FIG. 19A, illustrating radial geometric relationships between the ring electrode member 1906, the wire electrode member 1908, and the longitudinal centerline 1903. The longitudinal centerline 1903 passes through the center of the catheter inner body 1901. An inner radial distance 1932 extends from the longitudinal centerline 1903 to an inner boundary of the exposed discharge surfaces (e.g., a depth of cut or skive 719). An outer radial distance 1934 extends from the longitudinal centerline 1903 to an outer boundary of the exposed discharge surfaces (e.g., electrode bodies). A common radial envelope 1936 encompasses both the ring electrode member 1906 and the wire electrode member 1908, defined between the inner radial distance 1932 and the outer radial distance 1934.

As shown in FIG. 19D, both the ring electrode spark initiation location 1916 and the wire electrode spark initiation location 1918 are positioned at the same or substantially the same radial distance from the longitudinal centerline 1903, notwithstanding differences in electrode geometry or circumferential placement. As shown, the spark initiation locations 1916 and 1918 are co-radial or substantially co-radial relative to the longitudinal centerline 1903. The exposed discharge surfaces are limited to a same radial distance relative to the longitudinal centerline 1903, the common radial envelope 1936. Some embodiments may, for example, be implemented as described with reference to the compact radial packaging of the emitter module 116 of FIG. 1 and the PSSE module 316 of FIG. 3.

Together, FIGS. 19A-19D illustrate embodiments in which the ring electrode member 1906 and the wire electrode member 1908 include discrete spark initiation locations 1916 and 1918 that are positioned at a common radial distance from the longitudinal centerline 1903, bounded by insulative members 1910 and 1912, and constrained within a common radial envelope 1936 defined between inner radial distance 1932 and outer radial distance 1934. This co-radial, insulation-defined configuration may, for example, facilitate compact radial packaging, controlled spark initiation, and accommodation of a guidewire lumen sized for relatively large guidewires, including guidewires having diameters of between 0.025-inch (0.635 mm) to 0.095-inch (2.413 mm), or between about 0.025 inch (0.634 mm) and about 0.095 inch (2.413 mm), approximately or equal to 0.035 inch (0.889 mm), or larger, as described with reference to FIGS. 1 and 15.

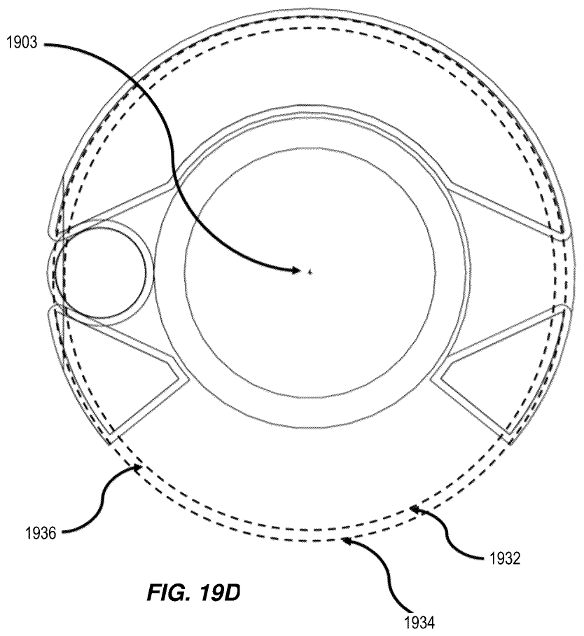

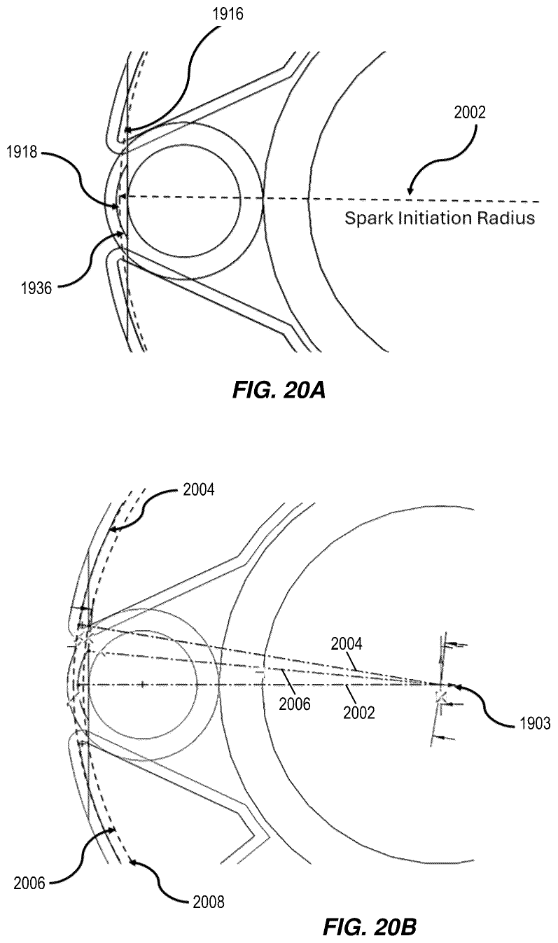

FIGS. 20A and 20B depict close-up cross-sectional views of the embodiment of FIG. 19A, illustrating example spark initiation radius geometrical relationships between the ring electrode member 1906 and the wire electrode member 1908 relative to the longitudinal centerline 1903. The views of FIGS. 20A and 20B may be taken at or near the same transverse plane as the cross-sectional views of FIGS. 19B-19D.

FIG. 20A depicts a close-up cross-sectional view illustrating the co-radial positioning of the ring electrode spark initiation location 1916 and the wire electrode spark initiation location 1918 relative to the longitudinal centerline 1903. As shown, the ring electrode spark initiation location 1916 and the wire electrode spark initiation location 1918 are each positioned at a common spark initiation radius 2002 relative to the longitudinal centerline 1903. The spark initiation locations 1916 and 1918 may be example implementations of the exposed discharge surfaces described herein.

The spark initiation radius 2002 extends from the longitudinal centerline 1903 to the spark initiation locations 1916 and 1918. As shown, both the ring electrode spark initiation location 1916 and the wire electrode spark initiation location 1918 (e.g., the exposed discharge surfaces) lie on the spark initiation radius 2002, notwithstanding differences in electrode body geometry or circumferential extent. The co-radial positioning of the spark initiation locations 1916 and 1918 on the spark initiation radius 2002 may, for example, be implemented with the co-radial electrode architecture described with reference to FIGS. 19A-19D, such as in the emitter module(s) (e.g., emitter module 116 of FIG. 1).

The co-radial positioning of the spark initiation locations 1916 and 1918 on the spark initiation radius 2002 may, for example, facilitate consistent electric field distribution between opposing spark initiation surfaces during application of a high-voltage pulse. Such co-radial positioning may, for example, contribute to predictable spark initiation behavior and may, for example, reduce variability in pressure wave generation across successive discharge events, as described with reference to the PSSE module 316 of FIG. 3.

FIG. 20B depicts a further close-up cross-sectional view of the embodiment of FIG. 19A, further illustrating the spark initiation radius 2002 and associated geometric relationships. As shown in FIG. 20B, the spark initiation radius 2002 extends from the longitudinal centerline 1903 to the spark initiation locations 1916 and 1918.

An outer radius 2004 extends from the longitudinal centerline 1903 to an outer boundary of the electrode bodies. An inner radius 2006 extends from the longitudinal centerline 1903 to an inner boundary of the electrode bodies. A common radial envelope 2008 is defined between the inner radius 2006 and the outer radius 2004. As shown, the spark initiation radius 2002 falls within the common radial envelope 2008, illustrating that the spark initiation locations 1916 and 1918 are contained within a common radial distance. The common radial envelope 2008 may, for example, be co-extensive with the common radial envelope 1936 described with reference to FIG. 19D. The common radial envelope 2008 may, for example, be contained within the common radial envelope 1924.

As shown in FIG. 20B, a circumference defined by spark initiation radius 2006 is defined by the spark initiation locations 1916 and 1918 as a diametric dimension passing through the longitudinal centerline 1903. The circumference defined by spark initiation radius 2006 may, for example, provide a geometric reference for characterizing the co-radial positioning of the spark initiation locations relative to the catheter centerline, such as independent of variations in electrode body shape, size, and/or circumferential extent.

Together, FIGS. 20A and 20B illustrate embodiments in which the ring electrode spark initiation location 1916 and the wire electrode spark initiation location 1918 are positioned at a common spark initiation radius 2002 relative to the longitudinal centerline 1903, contained within a common radial envelope 2008 defined between the inner radius 2006 and the outer radius 2004. This co-radial spark initiation geometry may, for example, facilitate compact radial packaging of the emitter module, such as described with reference to FIGS. 1, 6B, and 19A-19D, and may, for example, contribute to accommodation of a guidewire lumen sized for relatively large guidewires, including guidewires having diameters of between 0.025-inch (0.635 mm) to 0.095-inch (2.413 mm), or between about 0.025 inch (0.635 mm) and about 0.095 inch (2.413 mm), approximately or equal to 0.035 inch (0.889 mm), or larger.

The co-radial spark initiation geometry illustrated in FIGS. 20A and 20B may, by way of example and not limitation, provide advantages relative to non-co-radial spark initiation configurations, such as in one or more of the following respects. By constraining spark initiation regions to co-radial positions, the configuration may, for example, establish consistent electric field geometry across discharge events. Such embodiments may, for example, advantageously reduce variability in breakdown voltage and/or spark formation behavior across successive discharge cycles. The co-radial spark initiation configuration may, for example, maintain predictable discharge characteristics as electrode surfaces erode during device operation, such as when both electrode surfaces erode within the same radial distance rather than at different radial distances that would alter the effective field geometry over time. Co-radial placement of the spark initiation locations 1916 and 1918 may, for example, reduce variability in electric field distribution and plasma propagation, such as by constraining initial discharge formation to a consistent radial position relative to the longitudinal centerline 1903, independent of circumferential position or electrode body geometry. In some examples, in an advantage over electrode configurations that expose a cylindrical conductor surface extending across multiple radial distances from the catheter centerline—which may create variable electric field distributions depending on which portion of the exposed conductor surface participates in a given discharge event—the co-radial spark initiation configuration may, for example, reduce inconsistent breakdown voltages, unpredictable spark locations, and/or non-uniform pressure wave characteristics across successive pulses. The co-radial arrangement may, for example, offer advantages from axial or offset electrode arrangements, such as in that radial alignment is preserved even when electrode shapes differ, which may, for example, advantageously reduce rotational sensitivity of spark initiation behavior. The co-radial configuration may, for example, facilitate simplified manufacturing relative to concentric and/or fully circumferential electrode designs, such as enabling intentional diversification of discharge initiation locations. In advantages over IVL systems, such as in which discharge location vary stochastically and/or concentrates at a single erosion-prone site, the co-radial configuration may, for example, structurally enable repeatable redistribution of discharge initiation among multiple predefined locations. Such embodiments may, for example, advantageously reduce localized erosion and/or mitigate concentrated wear. Co-radial arrangement may, for example, maintain a more consistent effective spark gap over the operational lifespan of the emitter, such as relative to electrode configurations that concentrate erosion at limited surface regions. Such embodiments may, for example, advantageously reduce variation in discharge gap and/or associated variation in pressure wave characteristics.

In some embodiments, the spark initiation behavior of the device may be governed by an interdependent geometric relationship between the diameter of the wire electrode member 1908, the inner diameter of the ring electrode member 1906, the circumferential width of the ring electrode channel 1904, and the radial depth of insulation removal defining the exposed spark initiation locations 1916 and 1918. These parameters collectively define both a minimum edge-to-edge distance and a median electrode distance between opposing spark initiation sites, as further described with reference to FIGS. 23A-23B. The minimum edge-to-edge distance may, for example, determine a lower electrical breakdown threshold relative to the median electrode distance, such that spark initiation preferentially occurs at the nearest defined initiation surfaces. By geometrically encoding this gap hierarchy, the configuration may, for example, cause electrical discharge to initiate first at nearest sites prior to initiating at median sites at higher applied voltages. This may, for example, advantageously produce a deterministic and repeatable discharge sequence. By encoding voltage parameters into electrode geometry, rather than relying solely on real-time feedback, the configuration may, for example, enable operation within a constrained generator voltage range throughout device lifetime while maintaining consistent pressure wave output, such as discuss with reference to FIG. 16.

In some embodiments, the co-radial spark initiation geometry illustrated in FIGS. 20A and 20B may further support a progressive spark surface emitter architecture in which discharge location is governed by geometry rather than fixed electrode placement. Such embodiments may, for example, advantageously enable predictable energy delivery, extended emitter life, and/or compatibility with large guidewire lumens. The co-radial spark initiation radius 2002 may, for example, serve as a geometric primitive from which other design parameters—including minimum edge-to-edge distance, median electrode distance, channel width, and insulation removal depth—may be derived and controlled. Such geometric relationships may, for example, allow the emitter to be configured to maintain consistent pressure wave output across a large number of discharge cycles while operating within a limited generator voltage range, as described at least with reference to FIGS. 16 and 18.

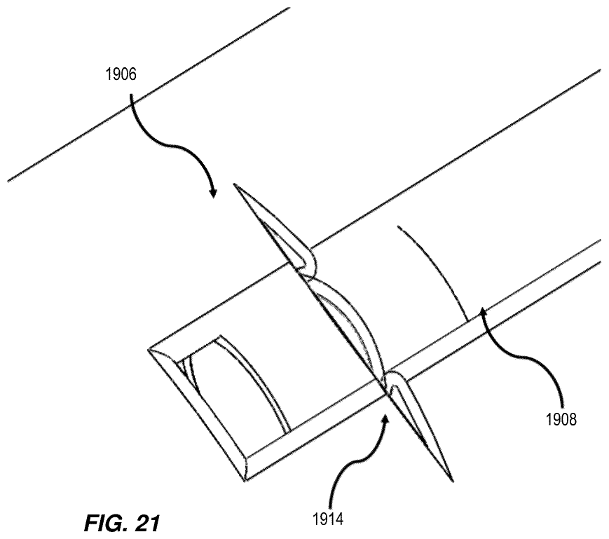

FIG. 21 depicts an example close-up perspective view of an example progressive spark surface emitter (PSSE) embodiment illustrating a spark initiation region between a ring electrode member 1906 and a wire electrode member 1908. The embodiment illustrated in FIG. 21 may be an example implementation of the emitter module 116 described with reference to FIG. 1 and/or the PSSE module 316 described with reference to FIG. 3.

As shown in FIG. 21, the ring electrode member 1906 is disposed circumferentially around the catheter inner body 1901. The wire electrode member 1908 is positioned within the ring electrode channel 1904 of the ring electrode member 1906. The ring electrode channel 1904 may be an example implementation of the conductor channel 421 described with reference to FIG. 4. The ring electrode insulative member 1910 terminates at the ring electrode spark initiation location 1916. The wire electrode insulative member 1912 terminates at the wire electrode spark initiation location 1918. The termination may, for example, be generated by a subtractive operation (e.g., cutting a notch). The initial spark generation region 1914 is defined by the ring electrode spark initiation location 1916 and the wire electrode spark initiation location 1918, within the ring electrode channel 1904.

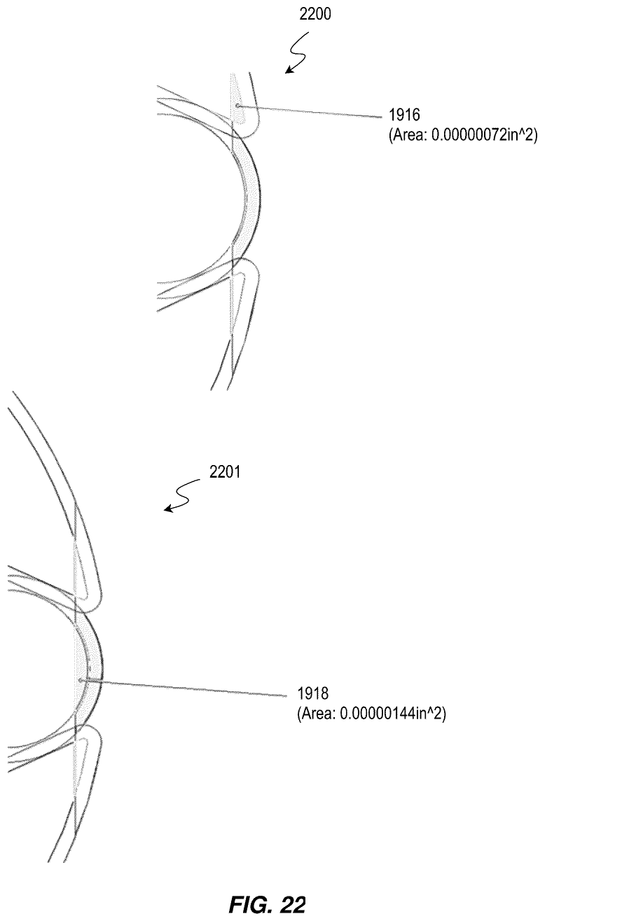

FIG. 22 depicts an example embodiment illustrating equal or substantially equal exposed discharge surface areas of the ring electrode member 1906 and the wire electrode member 1908, such as the embodiment of FIG. 21. As shown in FIG. 22, an exposed emitter body surface area 2200 is defined by the ring electrode spark initiation location 1916 within the discharge aperture. An exposed wire conductor surface area 2201 is defined by the wire electrode spark initiation location 1918 within the discharge aperture. As shown, the total exposed emitter body surface area 2200 and the exposed wire conductor surface area 2201 are equal or substantially equal in this example. The total exposed emitter body surface area is made up in the depicted example of two exposed emitter body surface area 2200, each at 0.00000072 in² (0.00045452 mm²), totaling 0.00000144 in² (0.000929203 mm²). In the depicted example, the exposed wire conductor surface area 2201 is 0.00000144 in² (0.000929203 mm²). The ring electrode insulative member 1910 and the wire electrode insulative member 1912 bound the respective exposed surfaces such that the exposed emitter body surface area 2200 and the exposed wire conductor surface area 2201 are each defined by the geometry of insulation termination rather than by the full extent of the underlying electrode bodies.

Equal or substantially equal exposed discharge surface areas, such as illustrated in FIG. 22, may, for example, promote symmetric charge distribution between the ring electrode member 1906 and the wire electrode member 1908 during application of a high-voltage pulse. Such symmetry may, for example, reduce variability in spark initiation location and/or increase repeatability of pressure wave output across successive discharge events.

In some embodiments, the ratio of the exposed emitter body surface area 2200 to the exposed wire conductor surface area 2201 may be less than or equal to 10:1, less than or equal to 5:1, less than or equal to 3:1, and/or equal or substantially equal to 1:1. A ratio closer to 1:1 may, for example, further reduce variability in charge distribution and spark initiation behavior. In some embodiments, the ratio may vary over multiple discharge events as electrode material erodes during repeated spark formation cycles.

FIGS. 23A and 23B depict a top plan view and a cross-section view, respectively, of the embodiment of FIG. 21, illustrating example geometric relationships related to spark gaps between the ring electrode member 1906 and the wire electrode member 1908. The ring electrode channel 1904 extends longitudinally along the ring electrode member 1906. The wire electrode member 1908 is disposed within the ring electrode channel 1904. The ring electrode insulative member 1910 and the wire electrode insulative member 1912 bound the spark initiation locations 1916 and 1918 within the ring electrode channel 1904.

A ring electrode nearest spark initiation site 2302 is defined on the ring electrode member 1906 at a location of closest proximity to the wire electrode member 1908 within the ring electrode channel 1904. A ring electrode median spark initiation site 2304 is defined on the ring electrode member 1906 at a median position of the ring electrode spark initiation location 1916 within the discharge aperture. A wire electrode median spark initiation site 2306 is defined on the wire electrode member 1908 at a median position of the wire electrode spark initiation location 1918 within the discharge aperture. A wire electrode nearest spark initiation site 2308 is defined on the wire electrode member 1908 at a location of closest proximity to the ring electrode member 1906 within the ring electrode channel 1904.

An initial spark depth of insulation removal distance 2310 is defined by the depth at which the ring electrode insulative member 1910 and/or the wire electrode insulative member 1912 terminate within the ring electrode channel 1904. The initial spark depth of insulation removal distance 2310 participates in defining the spark gap geometry at the initial spark generation region 1914, independently of the channel width of the ring electrode channel 1904.

An initial spark generation first edge-to-edge distance 2312 is defined between the ring electrode nearest spark initiation site 2302 and the wire electrode nearest spark initiation site 2308. An initial spark generation second edge-to-edge distance 2314 is defined between the ring electrode median spark initiation site 2304 and the wire electrode median spark initiation site 2306. As shown, the initial spark generation first edge-to-edge distance 2312 is less than the initial spark generation second edge-to-edge distance 2314. This geometric hierarchy may, for example, cause electrical discharge to initiate preferentially at the nearest spark initiation sites 2302 and 2308 before propagating to the median spark initiation sites 2304 and 2306 at higher applied voltages, such as consistent with the (e.g., deterministic) spark initiation geometry described with reference to FIGS. 19A-20B.

The channel width of the ring electrode channel 1904 constrains lateral plasma expansion during electrical discharge independently of the initial spark generation first edge-to-edge distance 2312. The initial spark depth of insulation removal distance 2310 participates in defining the initial spark generation first edge-to-edge distance 2312 independently of the channel width.

A spark progression path is defined along the ring electrode channel 1904 in the longitudinal direction, extending from the initial spark generation region 1914 toward a distal end of the ring electrode channel 1904. During repeated discharge events, electrode material at the wire electrode spark initiation location 1918 erodes, causing the effective spark initiation location to migrate along the spark progression path. The geometry of the ring electrode channel 1904 and the insulation termination may be selected such that the initial spark generation first edge-to-edge distance 2312 remains constant or substantially constant as erosion progresses along the spark progression path, consistent with the spark progression geometry described with reference to FIGS. 3 and 6A.

FIG. 23B depicts a cross-section view of the embodiment of FIG. 21, further illustrating the geometric relationships of FIG. 23A in the transverse plane. As shown, the ring electrode member 1906 is disposed circumferentially around the catheter inner body 1901. The wire electrode member 1908 is positioned within the ring electrode channel 1904. The ring electrode insulative member 1910 and the wire electrode insulative member 1912 bound the spark initiation locations 1916 and 1918.

As shown in FIG. 23B, the initial spark generation first edge-to-edge distance 2312 extends between the ring electrode nearest spark initiation site 2302 and the wire electrode nearest spark initiation site 2308 at their closest point of opposition. The initial spark generation second edge-to-edge distance 2314 extends between the ring electrode median spark initiation site 2304 and the wire electrode median spark initiation site 2306. The initial spark depth of insulation removal distance 2310 is defined by the depth at which the insulative members 1910 and 1912 terminate within the ring electrode channel 1904.

In some embodiments, the initial spark generation first edge-to-edge distance 2312 may be defined by: (i) the diameter of the wire electrode member 1908, (ii) the inner diameter of the ring electrode member 1906, (iii) the initial spark depth of insulation removal distance 2310, and (iv) the channel width of the ring electrode channel 1904.

Together, FIGS. 23A and 23B illustrate example geometric relationships between the ring electrode nearest spark initiation site 2302, the ring electrode median spark initiation site 2304, the wire electrode median spark initiation site 2306, the wire electrode nearest spark initiation site 2308, the initial spark depth of insulation removal distance 2310, the initial spark generation first edge-to-edge distance 2312, and the initial spark generation second edge-to-edge distance 2314. The relationships may, for example, cooperate to define both the breakdown voltage threshold and the discharge current magnitude independently, consistent with the voltage-current decoupling architecture described herein. Such geometric interdependence may, for example, allow the emitter to be configured to maintain consistent pressure wave output across a large number of discharge cycles while operating within a limited generator voltage range, as described with reference to FIGS. 16 and 18.

In some embodiments, the exposed emitter body surface area 2200 and the exposed wire conductor surface area 2201 may be characterized as: A_{wire}=d_{wire}×Lₛₖᵢᵥₑ, where d_{wire} is the diameter of the wire electrode member 1908 and Lₛₖᵢᵥₑ is the longitudinal length of the cut or skive defining the exposed wire electrode spark initiation location 1918.

The exposed emitter body surface area 2200 on each side of the ring electrode channel 1904 may be approximated as: A_{ring,side}=tᵢₙₛᵤₗₐₜᵢₒₙᵣₑₘₒᵥₐₗ×Lₛₖᵢᵥₑ, where tᵢₙₛᵤₗₐₜᵢₒₙᵣₑₘₒᵥₐₗ is the depth of insulation removal defining the ring electrode spark initiation location 1916 on each side of the ring electrode channel 1904.

The total exposed emitter body surface area 2200 may be approximated as: A_{ring,total}=2×tᵢₙₛᵤₗₐₜᵢₒₙᵣₑₘₒᵥₐₗ×Lₛₖᵢᵥₑ, such that the ratio of total exposed emitter body surface area 2200 to exposed wire conductor surface area 2201 may be expressed as:

$$\frac{{A}_{ring , total}}{{A}_{wire}} = \frac{2 \times {t}_{insulationremoval}}{{d}_{wire}} .$$

This relationship illustrates that the surface area ratio may be independently controlled by selecting the depth of insulation removal tᵢₙₛᵤₗₐₜᵢₒₙᵣₑₘₒᵥₐₗ relative to the wire electrode diameter d_{wire}, such as without changes to the longitudinal skive length Lₛₖᵢᵥₑ. For example, setting

$${t}_{insulationremoval} = \frac{{d}_{wire}}{2}$$

may, for example, produce a surface area ratio of approximately or equal to 1:1, such as illustrated in FIG. 22. Such geometric control of the surface area ratio may, for example, facilitate symmetric charge distribution between the ring electrode member 1906 and the wire electrode member 1908.

In some embodiments, the initial spark generation first edge-to-edge distance 2312 may be characterized mathematically. For example, the initial spark generation first edge-to-edge distance 2312 may be approximated as:

$${d}_{1} = \frac{{ID}_{ring} - {d}_{wire}}{2} - {t}_{insulationremoval} ,$$

where ID_{ring} is the inner diameter of the ring electrode member 1906, d_{wire} is the diameter of the wire electrode member 1908, and tᵢₙₛᵤₗₐₜᵢₒₙᵣₑₘₒᵥₐₗ is the initial spark depth of insulation removal distance 2310. This relationship illustrates that the initial spark generation first edge-to-edge distance 2312 may be controlled (e.g., independently) by selecting the inner diameter of the ring electrode member 1906, the wire electrode diameter, and the depth of insulation removal, such as without changes to the channel width of the ring electrode channel 1904.

The initial spark generation second edge-to-edge distance 2314 between the ring electrode median spark initiation site 2304 and the wire electrode median spark initiation site 2306 may be approximated as:

$${d}_{2} = \sqrt{{( \frac{{W}_{channel}}{2} )}^{2} + {( \frac{{ID}_{ring} - {d}_{wire}}{2} - {t}_{insulationremoval} )}^{2}} ,$$

where W_{channel} is the channel width of the ring electrode channel 1904. As shown, the initial spark generation second edge-to-edge distance 2314 is greater than the initial spark generation first edge-to-edge distance 2312 because the median spark initiation sites 2304 and 2306 are laterally offset from the nearest spark initiation sites 2302 and 2308 by

$$\frac{{W}_{channel}}{2}$$

or approximately

$$\frac{{W}_{channel}}{2}$$

in the circumferential direction. This geometric hierarchy may, for example, cause electrical discharge to initiate preferentially at the nearest spark initiation sites 2302 and 2308 defined by d₁ before propagating to the median sites 2304 and 2306 at higher applied voltages.

The channel width W_{channel} of the ring electrode channel 1904 may, for example, constrain (e.g., independently) lateral plasma expansion during electrical discharge. For example, reducing W_{channel} may, for example, increase discharge current confinement, such as without reducing the initial spark generation first edge-to-edge distance 2312. Reducing tᵢₙₛᵤₗₐₜᵢₒₙᵣₑₘₒᵥₐₗ may, for example, reduce the initial spark generation first edge-to-edge distance 2312 and thus reduce the breakdown voltage, such as without changes to W_{channel}.

FIG. 24 depicts a top plan view of an example PSSE embodiment illustrating geometric relationships related to spark gaps of an embodiment with a wider exposed area relative to the embodiment of FIG. 21. The embodiment illustrated in FIG. 24 may be an example implementation of the emitter module 116 described with reference to FIG. 1 and/or the PSSE module 316 described with reference to FIG. 3, configured with a wider (e.g., along the longitudinal axis) discharge aperture geometry.

As shown in FIG. 24, the ring electrode channel 1904 extends longitudinally along the ring electrode member 1906. The wire electrode member 1908 is disposed within the ring electrode channel 1904. A ring electrode nearest spark initiation site 2402 is defined on the ring electrode member 1906 at a location of closest proximity to the wire electrode member 1908 within the ring electrode channel 1904. A ring electrode median spark initiation site 2404 is defined on the ring electrode member 1906 at a median position of the ring electrode spark initiation location within the discharge aperture. A wire electrode median spark initiation site 2406 is defined on the wire electrode member 1908 at a median position of the wire electrode spark initiation location within the discharge aperture. A wire electrode nearest spark initiation site 2408 is defined on the wire electrode member 1908 at a location of closest proximity to the ring electrode member 1906 within the ring electrode channel 1904.

A first edge-to-edge distance 2412 is defined between the ring electrode nearest spark initiation site 2402 and the wire electrode nearest spark initiation site 2408. A median electrode distance 2414 is defined between the ring electrode median spark initiation site 2404 and the wire electrode median spark initiation site 2406. As shown, the first edge-to-edge distance 2412 is less than the median electrode distance 2414, consistent with the geometric hierarchy described with reference to FIGS. 23A-23B.

In the wider exposed area embodiment illustrated in FIG. 24, the depth of insulation removal may be greater than in the embodiment of FIG. 21, resulting in a reduced first edge-to-edge distance 2412 relative to the embodiment of FIG. 21, consistent with the mathematical relationship:

$${d}_{1} = \frac{{ID}_{ring} - {d}_{wire}}{2} - {t}_{insulationremoval} ,$$

described with reference to FIG. 23B. Such a configuration may, for example, reduce the breakdown voltage to initiate electrical discharge at the nearest spark initiation sites 2402 and 2408. The wider exposed area may, for example, affect the exposed discharge surface areas and associated charge distribution characteristics described with reference to FIG. 22.

FIG. 25 depicts a cross-section view of the wider exposed area embodiment of FIG. 24, further illustrating the geometric relationships in the transverse plane. The embodiment illustrated in FIG. 25 may be an example implementation of the emitter module 116 described with reference to FIG. 1 and/or the PSSE module 316 described with reference to FIG. 3.

As shown in FIG. 25, the ring electrode member 1906 is disposed circumferentially around the catheter inner body 1901. The wire electrode member 1908 is positioned within the ring electrode channel 1904. A channel width 2504 is defined by the opposing channel walls of the ring electrode channel 1904 bounding the wire electrode member 1908. The ring electrode member 2506 defines the annular geometry of the emitter module in this embodiment, including an outer surface and an inner surface. As shown, the wire electrode member 1908 and the ring electrode channel 1904 are positioned entirely within the volumetric shell bounded by the outer surface and the inner surface of the ring electrode member 2506, consistent with the compact radial packaging geometry described with reference at least to FIGS. 6B and 19C-19D.

A wider ring electrode spark initiation location 2516 is defined on the ring electrode member 1906 within the discharge aperture. A wider wire electrode spark initiation location 2518 is defined on the wire electrode member 1908 within the discharge aperture. The wider ring electrode spark initiation location 2516 and the wider wire electrode spark initiation location 2518 are each larger in exposed surface area than the corresponding spark initiation locations 1916 and 1918 of the embodiment of FIG. 21, such as due to the greater depth of insulation removal in this embodiment.

The channel width 2504 constrains lateral plasma expansion during electrical discharge independently of the first edge-to-edge distance 2412 between the ring electrode nearest spark initiation site 2402 and the wire electrode nearest spark initiation site 2408. Such independence may, for example, allow the breakdown voltage to be reduced, such as by increasing the depth of insulation removal without changes to the channel width 2504, and may, for example, allow the discharge current magnitude to be adjusted by changing the channel width 2504 without affecting the first edge-to-edge distance 2412.

FIG. 26 depicts a close-up cross-section view of the wider exposed area embodiment of FIGS. 24-25, further illustrating example geometric relationships at the spark initiation region. The embodiment illustrated in FIG. 26 may be an example implementation of the emitter module 116 described with reference to FIG. 1 and/or the PSSE module 316 described with reference to FIG. 3.

Together, FIGS. 24-26 illustrate that the geometric relationships between the ring electrode nearest spark initiation site 2602, the ring electrode median spark initiation site 2604, the wire electrode median spark initiation site 2606, the wire electrode nearest spark initiation site 2608, the initial spark depth of insulation removal distance 2610, the initial spark generation first edge-to-edge distance 2612, and the initial spark generation median electrode distance 2614 cooperate to define both the breakdown voltage threshold and the discharge current magnitude independently, consistent with the voltage-current decoupling architecture described herein. By varying the depth of insulation removal relative to the embodiment of FIG. 21, the wider exposed area embodiment may, for example, provide a different (e.g., lower) breakdown voltage threshold while maintaining independent control of discharge current magnitude through the channel width 2504 described with reference to FIG. 25.

FIGS. 27A and 27B depict a perspective view and a cross-section view, respectively, of an example PSSE embodiment with a shaped conductor channel illustrating conductor retentive shaping. The embodiment illustrated in FIGS. 27A and 27B may be an example implementation of the emitter module 116 described with reference to FIG. 1 and/or the PSSE module 316 described with reference to FIG. 3, configured with a shaped internal surface geometry of the conductor channel to mechanically retain the discharge conductor.

As shown in FIGS. 27A and 27B, the ring electrode member 1906 includes a wire electrode channel 2702 formed in the ring electrode member 1906. The wire electrode channel 2702 may be an example implementation of the ring electrode channel 1904 described with reference to FIGS. 19B-19D and the conductor channel 421 described with reference to FIG. 4. A wire electrode 2704 is disposed within the wire electrode channel 2702. The wire electrode 2704 may be an example implementation of the wire electrode member 1908 described with reference to FIGS. 19A-19D.

As shown in the cross-section view of FIG. 27B, the wire electrode channel 2702 includes a shaped internal surface such that a width of the wire electrode channel 2702 varies at different radial locations relative to the longitudinal centerline 1903. In the illustrated embodiment, the wire electrode channel 2702 is narrower at the outer surface of the ring electrode member 1906 than at a mid-depth location between the outer surface and the inner surface of the ring electrode member 1906. Such shaped internal surface geometry may, for example, mechanically retain the wire electrode 2704 within the wire electrode channel 2702, such as even without adhesive bonding and/or additional external support members. For example, some embodiments may be implementations of conductor retention geometry such as the conductor retaining surfaces 515 described with reference to FIG. 5.

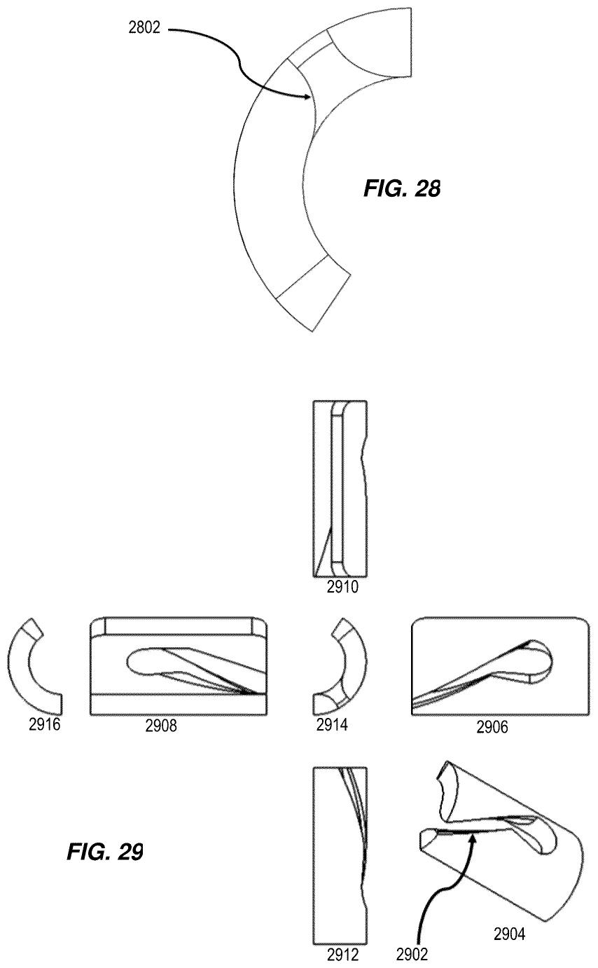

The shaped internal surface of the wire electrode channel 2702 may, for example, allow the wire electrode 2704 to be inserted radially into the wire electrode channel 2702 during assembly and retained by the narrower outer opening of the channel. Such retention may, for example, maintain alignment of the wire electrode 2704 relative to the ring electrode member 1906 during subsequent encapsulation and/or operation. In some embodiments, the shaped internal surface may include a curved and/or tapered profile, such as further described with reference to FIG. 28. The wire electrode 2704 and the wire electrode channel 2702 are positioned entirely within the volumetric shell bounded by the outer surface and the inner surface of the ring electrode member 1906, consistent with the compact radial packaging geometry described with reference to FIGS. 6B and 19C-19D.

FIG. 27C depicts a cross-section view of the embodiment of FIGS. 27A and 27B with an example wire electrode 2704 placed within the wire electrode channel 2702. As shown, the wire electrode 2704 is seated within the wire electrode channel 2702 such that the wire electrode 2704 is retained by the shaped internal surface of the wire electrode channel 2702. The wire electrode 2704 (e.g., such as wire electrode member 1908, conductor 412, conductor 112) is positioned such that its outermost surface is flush or substantially flush with, or recessed relative to, the outer surface of the ring electrode member 1906. Such flush or near-flush positioning may, for example, advantageously reduce or avoid localized radial protrusions that could otherwise increase a maximum outer diameter of the emitter module, such as consistent with the compact radial packaging geometry described with reference to FIGS. 1, 5, and 6B.

As shown in FIG. 27C, the narrower outer opening of the wire electrode channel 2702 retains the wire electrode 2704 within the channel, such as even without the channel fully enclosing the wire electrode 2704. Such partial capture may, for example, allow portions of the wire electrode 2704 to remain exposed for spark generation within a discharge aperture, such as described with reference to FIGS. 19C and 21. The shaped internal surface geometry illustrated in FIG. 27C may, for example, facilitate assembly of the emitter module, such as by allowing the wire electrode 2704 to be snapped and/or pressed radially into the wire electrode channel 2702 during manufacturing.

FIG. 28 depicts a close-up cross-section view of an example PSSE embodiment with a shaped conductor channel illustrating a curvilinear channel cross-section geometry. The embodiment illustrated in FIG. 28 may be an example implementation of the emitter module 116 described with reference to FIG. 1 and/or the shaped channel embodiment described with reference to FIGS. 27A-27C.

As shown in FIG. 28, a wire electrode channel 2802 is formed in the ring electrode member 1906. The wire electrode channel 2802 may be an example implementation of the wire electrode channel 2702 described with reference to FIGS. 27A-27C. The wire electrode channel 2802 includes a curvilinear internal surface defining a cross-sectional profile that varies in width at different radial locations relative to the longitudinal centerline 1903. In the illustrated embodiment, the curvilinear internal surface of the wire electrode channel 2802 defines a profile that is wider at a mid-depth location than at the outer surface of the ring electrode member 1906, such that the wire electrode channel 2802 retains a wire electrode disposed therein, such as even without the channel fully enclosing the wire electrode.

The curvilinear internal surface of the wire electrode channel 2802 may, for example, provide a smooth retention profile that distributes contact forces between the wire electrode and the ring electrode member 1906 over a larger surface area relative to a sharp-edged retention geometry. Such distribution may, for example, reduce stress concentrations at the wire electrode and/or the ring electrode member 1906 during assembly and/or operation. The curvilinear profile may also, for example, facilitate insertion of the wire electrode into the wire electrode channel 2802 during manufacturing by providing a gradually narrowing entry geometry rather than an abrupt step change in channel width, as further described with reference to FIG. 31.

In some embodiments, the curvilinear internal surface of the wire electrode channel 2802 may be selected to match or approximate the outer profile of the wire electrode, such as to maximize contact area between the wire electrode and the channel walls. Such matching may, for example, further distribute retention forces and may, for example, contribute to positional stability of the wire electrode within the wire electrode channel 2802 during repeated spark generation events. The wire electrode channel 2802 and any wire electrode disposed therein are positioned entirely within the volumetric shell bounded by the outer surface and the inner surface of the ring electrode member 1906, consistent with the compact radial packaging geometry described with reference to FIGS. 6B and 19C-19D.

FIG. 29 depicts various views of an example emitter body with a non-linear channel 2902, illustrating the three-dimensional geometry of the non-linear conductor channel from multiple orientations. The emitter body illustrated in FIG. 29 may be an example implementation of the emitter body 403 described with reference to FIG. 4 and/or the ring electrode member 1906 described with reference to FIGS. 19A-19D, configured with a non-linear conductor channel consistent with the non-linear conductor channel geometry described with reference to FIG. 11.

As shown in FIG. 29, a perspective view 2904 illustrates the overall three-dimensional geometry of the emitter body with the non-linear channel. The non-linear channel follows a path that deviates from a straight longitudinal or chordal alignment, such as a curvilinear path (e.g., curved then linear at a diagonal in this example) in the circumferential direction relative to the emitter body, consistent with the non-linear conductor channel described with reference to FIG. 11. In some embodiments, curvilinear may refer to a linear path non-parallel to the longitudinal axis.

Atop view 2906 and a bottom view 2908 illustrate the non-linear channel as seen from opposing axial directions along the longitudinal centerline. As shown in the top view 2906 and the bottom view 2908, the non-linear channel follows a path that extends circumferentially around the emitter body, such that the channel entry and exit positions are circumferentially offset relative to one another. Such circumferential offset may, for example, distribute spark initiation locations and electrode erosion along a non-linear path, as described with reference to FIG. 11.

A front view 2910 and a back view 2912 illustrate the non-linear channel as seen from opposing lateral directions perpendicular to the longitudinal centerline. As shown in the front view 2910 and the back view 2912, the non-linear channel follows a path that includes both longitudinal and circumferential components, such that the channel path is visible from lateral viewing angles as a curved and/or angled feature on the outer surface of the emitter body.

An end view 2914 and an opposing end view 2916 illustrate the non-linear channel as seen from opposing ends of the emitter body along the longitudinal axis. As shown in the end view 2914 and the opposing end view 2916, the non-linear channel opens at one or both ends of the emitter body at circumferentially offset positions, consistent with the helical and/or curvilinear path geometry described with reference to FIG. 11.

Together, the multiple views of FIG. 29 illustrate that the non-linear channel follows a three-dimensional path along the emitter body that includes both longitudinal and circumferential components. A discharge conductor disposed within the non-linear channel, such as the wire electrode 2704 described with reference to FIGS. 27A-27C, may deviate from a straight radial or chordal alignment. Such non-linear conductor channel geometry may, for example, distribute spark initiation locations and electrode erosion along a non-linear path. Such embodiments may, for example, advantageously extend emitter operational life and/or distribute pressure wave generation around a larger portion of the emitter circumference, such as described with reference to FIG. 11.

In some embodiments, FIG. 29 may be implemented in the embodiment shown in FIG. 11. For example, the conductor 1135 along the curvilinear path may be disposed in the non-linear channel 2902 of the emitter body shown in FIG. 29.

FIGS. 30A and 30B depict an overall perspective view and a close-up perspective view, respectively, of an example emitter body with curvilinear channel boundaries. The emitter body illustrated in FIGS. 30A and 30B may be an example implementation of the emitter body 403 described with reference to FIG. 4 and/or the ring electrode member 1906 described with reference to FIGS. 19A-19D, configured with a conductor channel having curvilinear boundaries that vary in width along a spark progression path.

As shown in FIG. 30A, the ring electrode member 1906 includes a ring electrode channel window 3002 and a ring electrode channel 3004 formed in the ring electrode member 1906. The ring electrode channel window 3002 defines a wider region of the conductor channel at an initial spark generation region. The ring electrode channel 3004 defines a narrower region of the conductor channel extending longitudinally away from the ring electrode channel window 3002 along the spark progression path. The ring electrode channel window 3002 may be an example implementation of the discharge aperture 405 described with reference to FIG. 4 and/or the discharge aperture 705 described with reference to FIG. 7. The ring electrode channel 3004 may be an example implementation of the conductor channel 421 described with reference to FIG. 4 and/or the wire electrode channel 2802 described with reference to FIG. 28.

As shown in the close-up perspective view of FIG. 30B, the boundary between the ring electrode channel window 3002 and the ring electrode channel 3004 is defined by curvilinear surfaces of the ring electrode member 1906. The channel width transitions from the wider ring electrode channel window 3002 to the progressively narrower ring electrode channel 3004 along the spark progression path. In the illustrated embodiment, the ring electrode channel 3004 becomes progressively narrower in the circumferential direction as the channel extends longitudinally away from the ring electrode channel window 3002. Such progressive narrowing may, for example, cause the channel width—and thus the lateral confinement of plasma during electrical discharge—to decrease as spark initiation locations migrate along the spark progression path during progressive electrode erosion. By encoding channel width variation into the geometry of the ring electrode channel 3004, the emitter body may, for example, be configured such that discharge current magnitude varies predictably along the spark progression path, such as even without changes to applied voltage and/or external control signals, such as consistent with the voltage-current decoupling architecture described herein.

In some embodiments, the channel width may be progressively wider along the spark progression path rather than narrower, which may, for example, cause discharge current magnitude to increase as erosion progresses. Such geometric encoding of current characteristics along the spark progression path may, for example, allow the emitter module to be configured to produce varying pressure wave output in a predetermined manner across successive discharge cycles, as described with reference to FIG. 16. In some embodiments, the channel width variation along the spark progression path may be selected in combination with a predetermined voltage schedule, such as described with reference to FIGS. 16 and 18, such that both voltage and channel width cooperate to maintain consistent pressure wave output across a large number of discharge cycles while operating within a limited generator voltage range.

The curvilinear boundary between the ring electrode channel window 3002 and the ring electrode channel 3004 may, for example, guide erosion of a wire electrode disposed within the ring electrode channel 3004 along the predetermined spark progression path. As electrode material erodes during repeated discharge events, the effective spark initiation location may migrate from the ring electrode channel window 3002 into the ring electrode channel 3004, following the curvilinear boundary as a guided erosion path, consistent with the spark progression geometry described with reference to FIGS. 3, 6A, and 23A-23B. The curvilinear boundary geometry may, for example, provide a smooth transition between the wider ring electrode channel window 3002 and the narrower ring electrode channel 3004, which may, for example, reduce abrupt changes in plasma confinement as the spark initiation location migrates along the spark progression path.

As shown in FIGS. 30A and 30B, the ring electrode channel window 3002 and the ring electrode channel 3004 are positioned entirely within the volumetric shell bounded by the outer surface and the inner surface of the ring electrode member 1906, consistent with the compact radial packaging geometry described with reference to FIGS. 6B and 19C-19D. Such positioning may, for example, allow the ring electrode member 1906 to define a maximum radial profile for both the spark-generating surfaces and the conductor channel, potentially facilitating accommodation of a guidewire lumen sized for relatively large guidewires, including guidewires having diameters of up to approximately or equal to 0.035 inch (0.889 mm) or larger, as described with reference to FIGS. 1 and 15.

In some embodiments, the relationship between channel width and discharge current magnitude may be characterized mathematically. Without being bound to a particular theory, the discharge current I following electrical breakdown may be approximated as proportional to the channel width W_{channel}: I∝W_{channel}, such that a narrower channel width W_{channel} at a given position along the spark progression path may, for example, produce a lower discharge current magnitude at that position, and a wider channel width may, for example, produce a higher discharge current magnitude. By encoding a progressive variation in W_{channel} along the ring electrode channel 3004, as illustrated in FIGS. 30A-30B, the emitter body may, for example, be configured such that discharge current magnitude varies predictably as spark initiation locations migrate along the spark progression path during progressive electrode erosion.

The breakdown voltage V_{breakdown} initiating electrical discharge may be approximated as proportional to the initial spark generation first edge-to-edge distance d₁. This may, for example, be consistent with a Paschen-type relationship for liquid-phase discharge:

$${V}_{breakdown} ∝ {d}_{1} = \frac{{ID}_{ring} - {d}_{wire}}{2} - {t}_{insulationremoval} ,$$

where ID_{ring} is the inner diameter of the ring electrode member 1906, d_{wire} is the diameter of the wire electrode member 1908, and tᵢₙₛᵤₗₐₜᵢₒₙᵣₑₘₒᵥₐₗ is the depth of insulation removal at the given longitudinal position along the spark progression path, as described with reference to FIGS. 23A-23B. As the spark initiation location migrates from the ring electrode channel window 3002 into the progressively narrower ring electrode channel 3004, the value of tᵢₙₛᵤₗₐₜᵢₒₙᵣₑₘₒᵥₐₗ may change if the depth of insulation removal varies along the path, thereby varying d₁ and the corresponding V_{breakdown}.

The acoustic pressure wave magnitude P generated by a spark event may be approximated as proportional to the energy E deposited during the discharge event:

$$P ∝ E = \frac{1}{2} {C V}^{2} ,$$

where C is the effective capacitance of the discharge circuit and V is the applied voltage at the time of breakdown. By independently controlling V_{breakdown} through d₁ and discharge current magnitude through W_{channel}, the geometry of the ring electrode channel window 3002 and ring electrode channel 3004 illustrated in FIGS. 30A-30B may, for example, allow the pressure wave magnitude P to be maintained within a target range across successive discharge events as erosion progresses along the spark progression path, such as even without changes to the applied voltage schedule or external control signals, such as consistent with the voltage-current decoupling architecture of the emission cycle behavior described with reference to FIG. 16.

In some embodiments, the channel width variation along the spark progression path may be selected such that the product I*V_{breakdown}—and thus the instantaneous power deposited at the spark gap—remains constant or substantially constant as erosion progresses. For example, if V_{breakdown} increases as d₁ increases along the spark progression path, a corresponding increase in W_{channel} may, for example, increase discharge current magnitude proportionally, such that the deposited energy per discharge event remains constant or substantially constant. If W_{channel} decreases as erosion progresses, the reduced discharge current may, for example, be compensated by a corresponding increase in applied voltage according to a predetermined voltage schedule, such as described with reference to FIGS. 16 and 18. Such geometric and electrical cooperation may, for example, allow the emitter module to maintain consistent pressure wave output across a large number of discharge cycles while operating within a limited generator voltage range.

FIG. 31 depicts an example method 3100 of manufacturing an emitter module of an intravascular lithotripsy device, such as the emitter modules 116 described with reference to FIG. 1 and/or the PSSE modules 316 described with reference to FIG. 3. In the depicted example, the method 3100 is organized as three nested loops: an outer loop iterating over a number I of catheter shaft segments and/or emitter module positions, a middle loop iterating over a number J of emitter bodies within each segment, and an inner loop iterating over a number K of conductors within each emitter body.

At step 3102, counter variables i, j, and k are initialized to 1. The counter variables i, j, and k correspond to the current catheter shaft segment, emitter body, and conductor being processed, respectively, within the nested loop structure of the method 3100.

At step 3104, a catheter shaft segment is prepared for the i^{th} emitter module. Preparing the catheter shaft segment may, for example, include forming one or more shaft channels, such as the shaft channels 513 described with reference to FIG. 5, and/or other structural features configured to receive and support emitter bodies and conductors along the shaft. The catheter shaft may be an example implementation of the inner shaft 114 described with reference to FIG. 1 and/or the inner shaft 414 described with reference to FIG. 4.

At step 3106, the j^{th} emitter body is positioned on the catheter shaft. The emitter body may be an example implementation of the emitter body 403 described with reference to FIG. 4 and/or the ring electrode member 1906 described with reference to FIGS. 19A-19D. Positioning the emitter body may, for example, include placing the emitter body at a predetermined longitudinal location along the catheter shaft corresponding to a desired emitter module position relative to a treatment region, such as described with reference to FIGS. 13 and 15.

At step 3108, the k^{th} conductor is disposed within a conductor channel of the j^{th} emitter body. The conductor may be an example implementation of the wire electrode 2704 described with reference to FIGS. 27A-27C and/or the wire electrode member 1908 described with reference to FIGS. 19A-19D. The conductor channel may be an example implementation of the conductor channel 421 described with reference to FIG. 4, the wire electrode channel 2702 described with reference to FIGS. 27A-27C, and/or the ring electrode channel 3004 described with reference to FIGS. 30A-30B. In some embodiments, the conductor may be inserted radially into the conductor channel and retained by a shaped internal surface of the conductor channel, such as described with reference to FIGS. 27A-27C and FIG. 28. In some embodiments, the conductor may be disposed within a conductor channel that follows a non-linear path, such as described with reference to FIG. 11.

At decision step 3110, it is determined whether the conductor is correctly positioned within the conductor channel. If the conductor is not correctly positioned, the method proceeds to step 3112, at which the conductor is repositioned. Following step 3112, the method returns to step 3108. If the conductor is correctly positioned, the method proceeds to the next decision.

Following confirmation of correct conductor positioning at step 3110, it is determined at step 3114 whether k is less than or equal to K—that is, whether additional conductors remain to be disposed within the j^{th} emitter body. If so, the counter k is incremented to k+1 at step 3116 and the method returns to step 3108 to dispose the next conductor. If all K conductors have been disposed and correctly positioned, the method proceeds to step 3118.

At step 3118, the j^{th} emitter body is bonded to the catheter shaft. Bonding may, for example, include applying an adhesive including a photopolymerized adhesive with shadow curing properties, which may, for example, facilitate curing of adhesive in regions not directly exposed to a curing light source, such as regions beneath the emitter body and/or within conductor channels.

At decision step 3120, it is determined whether a subtractive forming method or a masked forming method is to be used to create the exposed discharge surfaces. If a subtractive method is selected, the method proceeds to step 3124, at which an encapsulating dielectric material is applied over the emitter body and conductors. The encapsulating dielectric material may be an example implementation of the capsule 301 described with reference to FIG. 3. The encapsulating dielectric material electrically isolates the emitter body from the conductors such that electrical discharge between the emitter body and the conductors is physically inhibited except at defined exposed discharge surfaces, consistent with the insulation-defined discharge region described herein. Following step 3124, the method proceeds to step 3126, at which a portion of the encapsulating dielectric material is removed to create the exposed discharge surfaces. Removal may, for example, include laser ablation, mechanical cutting, and/or chemical etching, such as described with reference to the cut or skive 719 of FIG. 7.

If a masked forming method is selected at decision step 3120, the method applies a mask at step 3122 and proceeds to step 3124, at which the encapsulating dielectric material is applied over the emitter body and conductors with a mask in place over the regions intended to remain exposed. Following step 3124, the method proceeds to step 3128, at which the mask is removed to reveal the exposed discharge surfaces. Such masked application may, for example, prevent deposition of the encapsulating dielectric material at the discharge aperture during application. For example, the cut or skive 719 may be formed by masking.

In both the subtractive and masked forming paths, the forming steps 3124, 3126 and/or 3128 may define one or more geometric relationships that govern spark initiation behavior and pressure wave output of the finished emitter module. For example, the forming steps may define an initial spark generation first edge-to-edge distance 2312 between the first exposed discharge surface and the second exposed discharge surface that is less than an initial spark generation second edge-to-edge distance 2314 at a laterally offset location within the discharge aperture, consistent with the geometric hierarchy described with reference to FIGS. 23A-23B. The forming steps may further define a ratio of a surface area of the first exposed discharge surface to a surface area of the second exposed discharge surface that is less than or equal to 10:1, and in some embodiments equal or substantially equal to 1:1, consistent with the surface area ratio geometry of FIG. 22. The forming steps may further define a predetermined spark progression path along which spark initiation locations migrate during progressive electrode erosion, consistent with the spark progression geometry described with reference to FIGS. 3, 6A, and 30A-30B.

At decision step 3130, it is determined whether j is less than or equal to J—that is, whether additional emitter bodies remain to be processed for the i^{th} emitter module. If so, the counter j is incremented to j+1 at step 3132 and the method returns to step 3106 to position the next emitter body. If all J emitter bodies have been processed, the method proceeds to decision step 3134.

At decision step 3134, it is determined whether i is less than or equal to I—that is, whether additional emitter modules remain to be prepared. If so, the counter i is incremented to i+1 at step 3136 and the method returns to step 3104 to prepare the catheter shaft for the next emitter module. If all I emitter modules have been processed, the method ends.

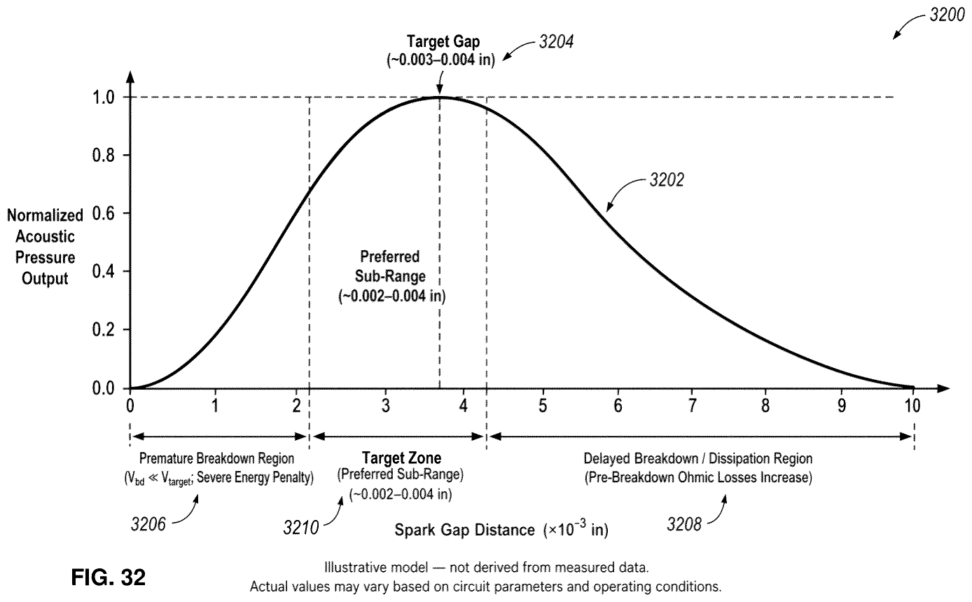

FIG. 32 depicts a plot 3200 of acoustic pressure output (Y-axis, normalized) versus spark gap distance (X-axis, in thousandths of an inch (in 25.4 micrometers), ranging from 0 to 10 thousandths of an inch (0 to 254 micrometers)) for an example IVL-class emitter circuit operating in physiological saline. As shown in FIG. 32, a curve 3202 illustrates the relationship between spark gap distance and acoustic pressure output, which may be non-monotonic (e.g., bell-shaped as shown). In this example, the curve 3202 shows a rising limb from 0 to approximately 2 to 3 thousandths of an inch (approximately 50.8 micrometers to 76.2 micrometers), a peak at approximately 3 to 4 thousandths of an inch (approximately 76.2 micrometers to 101.6 micrometers), and a falling limb from approximately 4 thousandths of an inch to 10 thousandths of an inch (approximately 101.6 micrometers to 254 micrometers). A target gap marker 3204 identifies the spark gap distance at which the breakdown voltage V_bd approximates the target circuit voltage V_target, corresponding to the peak of the curve 3202. A target zone 3210 identifies a range of spark gap distances surrounding the target gap marker 3204 within which acoustic pressure output may be at or near a peak value. A premature breakdown region 3206 identifies spark gap distances below the target zone 3210, in which V_bd may fall below V_target and available energy may be reduced. A delayed breakdown/dissipation region 3208 identifies spark gap distances above the target zone 3210, in which pre-breakdown ohmic dissipation may accumulate over an extended dwell time. The peak of the curve 3202 may correspond to the spark gap distance at which the breakdown voltage V_bd approximates the target circuit voltage V_target, consistent with the Lambda approximately equal to 1 matching condition described herein. Spark gap distances both smaller and larger than the peak distance may produce reduced acoustic pressure output, such as described elsewhere herein.

In some embodiments, the spark gap distance between the first exposed discharge surface and the second exposed discharge surface may be selected to influence acoustic pressure wave output. As described herein, the spark gap distance may influence the voltage-rise behavior at the emitter, which may influence the magnitude and timing of the generated pressure wave. In some embodiments, shorter spark gap distances may advantageously initiate electrical discharge earlier at a lower peak emitter voltage. In some embodiments, longer spark gap distances may advantageously allow higher stored potential to accumulate before breakdown. In some embodiments, there may be an empirically observed non-monotonic relationship between spark gap distance and acoustic pressure output, such as illustrated by the curve 3202 of the plot 3200 described with reference to FIG. 32. Without being bound to a particular theory, some embodiments may implement a mid-range balance, for example, at approximately 3 to 4 thousandths of an inch (approximately 76.2 micrometers to 101.6 micrometers), such as within the target zone 3210 identified with reference to FIG. 32, such as for IVL-class circuits operating in physiological saline at target voltages of approximately 2,000 V to 4,200 V.

In some embodiments, the spark gap distance may be between 0 inches (contact) and 0.010 inches (254 micrometers). In some embodiments, the spark gap distance may be between 0.001 inches (25.4 micrometers) and 0.006 inches (152.4 micrometers). In some embodiments, the spark gap distance may be between 0.002 inches (50.8 micrometers) and 0.005 inches (127 micrometers). In some embodiments, the spark gap distance may be between 0.003 inches (76.2 micrometers) and 0.004 inches (101.6 micrometers). In some embodiments, the spark gap distance may be less than 0.001 inches (25.4 micrometers), including sub-thousandth of an inch (sub-25.4 micrometer) gap distances at which discharge may still occur. For example, 0 inches to sub-thousandth of an inch (0 to sub-25.4 micrometers) gap distances may still achieve a gap in practice through conductivity barriers such as resistance at a junction of different materials. By way of example and not limitation, sparks across sub-thousandth of an inch (sub-25.4 micrometer) gaps may be achieved via oxide film breakdown and asperity micro-gaps.

In some embodiments, at nominally zero spark gap distance, such as when the emitter body and the discharge conductor are in crimped or welded contact, electrical discharge may still occur. In some embodiments, at nominally zero gap distance, the effective discharge path may be governed by oxide film thickness and asperity micro-gaps at the contact interface. In some embodiments, the oxide films may include chromium oxide (Cr2O3) on stainless steel surfaces and/or copper oxide (CuO) on copper surfaces. In some embodiments, the oxide films may have nanometer-scale thicknesses. In some embodiments, the actual contact area between nominally contacting metallic surfaces may be less than approximately 1% of the apparent contact area. In some embodiments, current densities at true contact points may be on the order of 10 to the seventh power amperes per square centimeter, which may be sufficient to vaporize metal asperities at the contact interface. In some embodiments, acoustic output from zero-gap discharge events may be spatially unpredictable and electrically uncontrolled.

In some embodiments, at spark gap distances below approximately 0.001 inches (25.4 micrometers), the electric field intensity across the spark gap may exceed the intrinsic electrical breakdown threshold of the conductive liquid medium. In some embodiments, the electrical breakdown threshold of physiological saline may be approximately 65 to 70 megavolts per meter (MV/m). In some embodiments, at spark gap distances below approximately 0.001 inches (25.4 micrometer) and at target voltages of approximately 3,500 V, the electric field intensity may be sufficiently high that direct liquid-phase streamer breakdown may occur. In some embodiments, direct liquid-phase streamer breakdown may bypass the vapor bubble formation phase that may otherwise precede plasma channel formation at larger spark gap distances. In some embodiments, bypassing the vapor bubble formation phase may result in near-instantaneous breakdown with a breakdown voltage V_bd substantially below V_target.

In some embodiments, the electric field intensity across the spark gap at a given target voltage may vary as a function of spark gap distance. In some embodiments, at a target voltage of approximately 3,500 V, the electric field intensity may be approximately 137.8 kV/mm at a spark gap distance of 0.001 inches (25.4 micrometers). In some embodiments, the electric field intensity may be approximately 68.9 kV/mm at a spark gap distance of 0.002 inches (50.8 micrometers). In some embodiments, the electric field intensity may be approximately 45.9 kV/mm at a spark gap distance of 0.003 inches (76.2 micrometers). In some embodiments, the electric field intensity may be approximately 34.5 kV/mm at a spark gap distance of 0.004 inches (101.6 micrometers). In some embodiments, the electric field intensity values described herein may be computed from a static field model (E=V/d) and may represent approximate values. In some embodiments, the actual breakdown behavior in a conductive liquid medium may differ from the static field model due to factors including electrode geometry, liquid conductivity, temperature, and pre-breakdown current flow.

Without being bound to a particular theory, peak acoustic pressure output may be achieved at an interior optimal spark gap distance at which a breakdown voltage V_bd across the spark gap approximates a target circuit voltage V_target, such that energy stored in the discharge circuit achieves a maxima at the moment of discharge. This matching condition may be characterized as V_bd approximately equal to V_target. This may, for example, be expressed as a dimensionless parameter Lambda. A target Lambda may, for example, be approximately equal to 1, where Lambda equals V_bd divided by V_target. When the spark gap distance is smaller than the ‘optimal’ distance, V_bd may fall below V_target. For example, the energy available for acoustic pressure wave generation may be reduced by a factor with a relationship of (V_bdN_target) squared. For example, a gap that breaks down at 2,000 V when the circuit is charged to 3,500 V may deliver merely approximately 33% of the intended stored energy, as (2,000/3,500) squared is approximately 0.33.

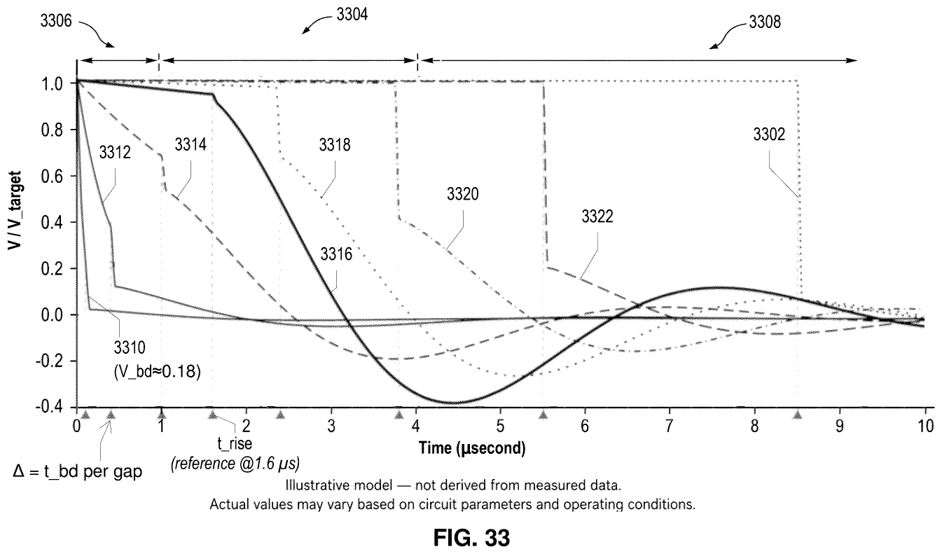

When the spark gap distance is larger than the ‘optimal’ distance, breakdown may be delayed beyond the peak of the circuit current waveform. For example, pre-breakdown ohmic dissipation may accumulate over the extended dwell time. Accumulated dissipation may, for example, reduce post-breakdown oscillation amplitude and/or acoustic output. Both mechanisms may, for example, reduce acoustic pressure output relative to the interior optimum, corresponding respectively to the premature breakdown region 3306 and the delayed breakdown region 3308 identified with reference to FIG. 33, and the premature breakdown region 3206 and the delayed breakdown/dissipation region 3208 identified with reference to FIG. 32. This reduction may, for example, produce the observed bell-shaped relationship between spark gap distance and pressure output illustrated by the curve 3202 of the plot 3200.

Without being bound to a particular theory, acoustic pressure output generated by a spark event may be related to the integral of instantaneous power delivered to the plasma channel during the impulsive discharge phase. In some embodiments, the instantaneous power P(t) delivered to the plasma channel at time t may be expressed as P(t)=V(t)*I(t), where V(t) is the voltage across the spark gap at time t and I(t) is the current flowing through the plasma channel at time t. In some embodiments, the total energy delivered to the plasma channel during the impulsive phase may be proportional to the

$${\int }_{t 0}^{t 1} P ( t )$$

over the duration of the discharge pulse (t0 to t1 in this example). In some embodiments, increasing the integral of V(t) times I(t) over the discharge pulse may, for example, increase the acoustic pressure output of the emitter module.

In some embodiments, increasing the integral of instantaneous power may involve satisfying multiple (e.g., two, more than two) conditions (e.g., simultaneously). A first condition may be that the breakdown voltage V_bd approximates the target circuit voltage V_target, such that the stored energy in the discharge circuit is at or near a maxima (e.g., local, global) at the moment the plasma channel forms. A second condition may be that the dimensionless parameter Lambda approximates 1, such that the plasma channel finishes forming at or nearly at the same time that the circuit current I(t) reaches its peak. In some embodiments, neither condition alone may be sufficient to increase acoustic pressure output. In some embodiments, a high breakdown voltage at a poorly timed channel formation may result in energy loss to pre-breakdown ohmic dissipation, such as in the delayed regime gap distances described herein. In some embodiments, a fast channel formation at a low breakdown voltage may result in energy loss. The energy loss may, for example, be to the premature breakdown penalty described herein, such as in the premature regime gap distances described herein. In some embodiments, the satisfaction of multiple conditions may, for example, advantageously produce a coincidence of high gap voltage and peak circuit current, which may advantageously increase the integral of instantaneous power during the impulsive phase.

In some embodiments, the rate of voltage collapse following breakdown (dV/dt) may not be a reliable indicator of acoustic pressure output. In some embodiments, a smaller spark gap distance may exhibit a steeper post-breakdown voltage collapse (higher magnitude dV/dt) than a larger spark gap distance. In some embodiments, a steeper post-breakdown voltage collapse may indicate higher instantaneous current flowing from the capacitor into the plasma channel. However, in some embodiments, the instantaneous power P(t)=V(t) times I(t) may be low even when dV/dt is steep, such as when the voltage V(t) at the moment of breakdown is substantially below V_target. For example, in the illustrative model of FIG. 33, the 0.001 inch (25.4 micrometer) gap distance curve 3312 may exhibit the steepest post-breakdown voltage collapse of the depicted curves, but may produce among the lowest acoustic output because the breakdown voltage ratio V_bdN_target is approximately 0.38, such that the voltage is low at the moment the steep collapse occurs, consistent with the premature breakdown region annotation 3306. In some embodiments, the 0.003 inch (76.2 micrometer) gap distance curve 3316 may exhibit a less steep post-breakdown voltage collapse than the 0.001 inch (25.4 micrometer) gap distance curve 3312, but may produce the highest acoustic output because the breakdown voltage ratio V_bd/V_target is approximately 0.94 and the breakdown timing coincides with the peak of the circuit current waveform, consistent with the target zone 3304.

In some embodiments, the spark gap distance may be selected such that the breakdown voltage V_bd is within 50% of the target circuit voltage V_target. In some embodiments, the spark gap distance may be selected such that V_bd is within 25% of V_target. In some embodiments, the spark gap distance may be selected such that V_bd is within 10% of V_target. A spark gap distance satisfying the condition V_bd approximately equal to V_target may, for example, increase the fraction of stored circuit energy available for conversion to acoustic pressure wave energy at the moment of discharge.

In some embodiments, the spark gap distance may be selected based on one or more circuit parameters including applied voltage, capacitance of the discharge circuit, inductance of the conductor set, and resistance of the conductor set. In some embodiments, the spark gap distance may be selected from a range of 0.001 inches (25.4 micrometers) to 0.005 inches (127 micrometers), such as, by way of example and not limitation, for IVL-class circuits operating at target voltages of approximately 2,000 V to 4,200 V in physiological saline. In some embodiments, the spark gap distance may be approximately 0.003 inches (76.2 micrometers) as an example initial design point for such circuits.

In some embodiments, the spark gap distance may be physically inspectable from the finished emitter module, such as by cross-sectional measurement of the distance between the first exposed discharge surface and the second exposed discharge surface.

FIG. 33 depicts a family of voltage-time traces (VN_target on the Y-axis versus time in microseconds on the X-axis) for spark gap distances ranging from 0.0005 inches to 0.010 inches (12.7 micrometers to 254 micrometers), for an example IVL-class emitter circuit. As shown in FIG. 33, each curve may begin at VN_target equal to 1.0 at time zero, representing the fully charged circuit. A circuit rise time reference line 3302 is shown at approximately 1.6 microseconds, corresponding to the circuit rise time t_rise for the illustrated example circuit model. A target zone 3304 identifies a range of spark gap distances within which breakdown timing may coincide with or approximate the circuit rise time t_rise. A premature breakdown region annotation 3306 identifies spark gap distances below the target zone 3304, in which breakdown may occur before the circuit current waveform reaches its peak. A delayed breakdown region annotation 3308 identifies spark gap distances above the target zone 3304, in which breakdown may occur after the circuit current waveform has passed its peak. In some embodiments, smaller spark gap distances may exhibit earlier breakdown timing, lower breakdown voltage ratios V_bdN_target, and/or reduced post-breakdown oscillation amplitude. In some embodiments, larger spark gap distances may exhibit later breakdown timing, higher pre-breakdown voltage retention, and/or reduced post-breakdown oscillation amplitude (e.g., due to accumulated pre-breakdown ohmic dissipation). In some embodiments, the curve corresponding to approximately 0.003 inches (76.2 micrometer) (e.g., the 0.003 inch (76.2 micrometer) gap distance curve 3316) may exhibit breakdown timing coinciding with the circuit rise time reference line 3302 at approximately 1.6 microseconds, within the target zone 3304, the highest post-breakdown oscillation amplitude, and the largest negative voltage excursion. This coincidence may, for example, be consistent with energy delivery maxima at the Lambda approximately equal to 1 matching condition. The figure is illustrative of the physical relationships described herein and is based on a representative circuit model (C approximately 100 nF, L approximately 10 microhenries); actual values may vary based on circuit parameters.

In some embodiments, the voltage-time traces depicted in FIG. 33 may be characterized by gap-specific breakdown timing, post-breakdown oscillation amplitude, and/or regime classification. In some embodiments, the 0.0005 inch (12.7 micrometer) gap distance, corresponding to the 0.0005 inch (12.7 micrometer) gap distance curve 3310, may be classified as an extreme premature breakdown regime. In some embodiments such as shown, the 0.0005 inch (12.7 micrometer) gap distance curve 3310 may exhibit breakdown at approximately 0.12 microseconds with a breakdown voltage ratio V_bd/V_target of approximately 0.18, within the premature breakdown region annotation 3306. As shown, the post-breakdown oscillation amplitude for the 0.0005 inch (12.7 micrometer) gap distance curve 3310 may be approximately 0.04, which may represent a near-negligible acoustic output condition.

In some embodiments, the 0.001 inch (25.4 micrometer) gap distance, corresponding to the 0.001 inch (25.4 micrometer) gap distance curve 3312, may be classified as a deep premature breakdown regime. In some embodiments, such as shown, the 0.001 inch (25.4 micrometer) gap distance curve 3312 may exhibit breakdown at approximately 0.4 microseconds with a breakdown voltage ratio V_bd/V_target of approximately 0.38. As shown, the post-breakdown oscillation amplitude for the 0.001 inch (25.4 micrometer) gap distance curve 3312 may be substantially reduced relative to the 0.003 inch (76.2 micrometer) gap distance curve 3316.

In some embodiments, the 0.002 inch (50.8 micrometer) gap distance, corresponding to the 0.002 inch (50.8 micrometer) gap distance curve 3314, may be classified as a near premature breakdown regime. In the depicted example, the 0.002 inch (50.8 micrometer) gap distance curve 3314 exhibits breakdown at approximately 1.0 microsecond.

In some embodiments, the 0.003 inch (76.2 micrometer) gap distance, corresponding to the 0.003 inch (76.2 micrometer) gap distance curve 3316, may be classified as an ‘optimal’ regime corresponding to the Lambda approximating an equal to 1 matching condition. In this example shown, the 0.003 inch (76.2 micrometer) gap distance curve 3316 exhibits breakdown at approximately 1.6 microseconds, coinciding with the circuit rise time t_rise indicated by the circuit rise time reference line 3302, within the target zone 3304. In some embodiments, such as shown, the 0.003 inch (76.2 micrometer) gap distance curve 3316 may exhibit the highest post-breakdown oscillation amplitude of the depicted gap distances, with a first peak of approximately 0.92 and a negative voltage excursion of approximately negative 0.35 times V_target.

In some embodiments, the 0.004 inch (101.6 micrometer) gap distance, corresponding to the 0.004 inch (101.6 micrometer) gap distance curve 3318, may be classified as an entering delayed regime. In the depicted example, the 0.004 inch (101.6 micrometer) gap distance curve 3318 exhibits breakdown at approximately 2.5 microseconds.

In some embodiments, the 0.006 inch (152.4 micrometer) gap distance, corresponding to the 0.006 inch (152.4 micrometer) gap distance curve 3320, may be classified as a delayed/degraded regime. In this particular example, the 0.006 inch (152.4 micrometer) gap distance curve 3320 exhibits breakdown at approximately 3.8 microseconds with a post-breakdown oscillation amplitude of approximately 0.42.

In some embodiments, the 0.008 inch (203.2 micrometer) gap distance, corresponding to the 0.008 inch (203.2 micrometer) gap distance curve 3322, may be classified as a marginal regime. As shown in this example, the 0.008 inch (203.2 micrometer) gap distance curve 3322 exhibits breakdown at approximately 5.5 microseconds with a post-breakdown oscillation amplitude of approximately 0.22.

In some embodiments, the 0.010 inch (254 micrometer) gap distance may be classified as a deep delayed/misfire regime. As shown for this illustrative example, the 0.010 inch (254 micrometer) gap distance exhibits breakdown at approximately 8.5 microseconds with a post-breakdown oscillation amplitude of approximately 0.08. In some embodiments, a post-breakdown oscillation amplitude of approximately 0.08 or less may, for example, correspond to an insufficient acoustic output condition for calcium fracture in a clinical application.

In some embodiments, regime classifications such as described herein may represent a continuum of discharge behavior as a function of spark gap distance. In some embodiments, the premature breakdown regimes (e.g., extreme premature, deep premature, near premature) may be characterized by breakdown occurring before the circuit current waveform reaches its peak. The energy available at the moment of discharge may, for example, be reduced relative to the target stored energy.

In some embodiments, the delayed regimes (e.g., entering delayed, delayed/degraded, marginal, deep delayed/misfire) may be characterized by breakdown occurring after the circuit current waveform has passed its peak. The pre-breakdown ohmic dissipation may, for example, accumulate over an extended dwell time.

In some embodiments, the ‘optimal’ regime may be characterized by breakdown timing that coincides with the peak of the circuit current waveform. For example, both the voltage at breakdown and the circuit current at breakdown may be at or near their respective maxima.

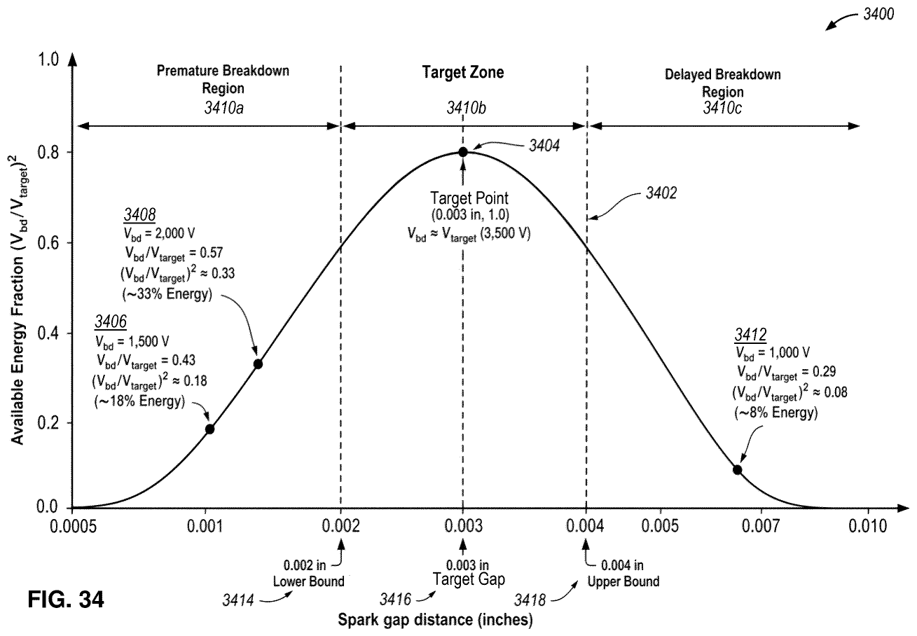

FIG. 34 depicts an energy penalty diagram 3400 showing an energy penalty curve 3402 of (V_bd/V_target) squared as a function of spark gap distance, illustrating the quadratic reduction in available stored energy for sub-optimal gap distances at which V_bd is less than V_target. A target point 3404 identifies the spark gap distance at which V_bd approximates V_target (e.g., approximately 3,500 V in the illustrated example) in this example. As shown, this point may correspond to an available energy fraction of approximately 1.0. Region labels 3410 identify a premature breakdown region 3410a at spark gap distances below a lower bound marker 3414, a target sub-range 3410b between the lower bound marker 3414 and an upper bound marker 3418 with a target gap marker 3416 therebetween, and a delayed breakdown region 3410c at spark gap distances above the upper bound marker 3418. In some embodiments, as shown, representative values along the energy penalty curve 3402 may include: at V_bdN_target equal to 0.57 (corresponding to V_bd equal to 2,000 V at V_target equal to 3,500 V), as identified by a second data point 3408, the available energy fraction may be approximately 33%; at V_bdN_target equal to 0.43, as identified by a first data point 3406, the available energy fraction may be approximately 18%; at V_bdN_target equal to 0.29, the available energy fraction may be approximately 8%, as shown at a third data point 3412.

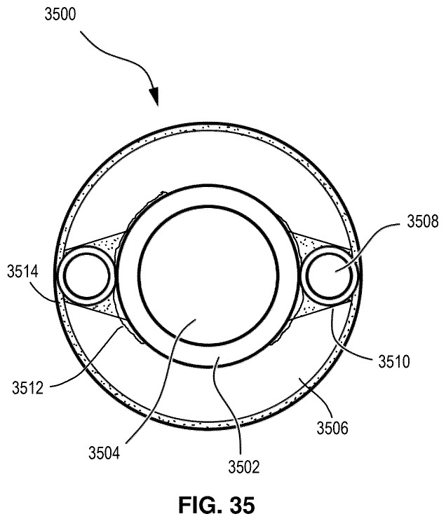

FIG. 35 depicts a cross-sectional view of an example emitter module assembly 3500, illustrating example geometric and material relationships between an inner catheter body 3502, a lumen 3504 defined by the inner catheter body 3502, an emitter body 3506, a conductor 3508, an insulator 3510, an adhesive 3512, and an encapsulating material 3514. The emitter module assembly 3500 may be an example implementation of the emitter module 116 described with reference to FIG. 1, the PSSE module 316 described with reference to FIG. 3, and/or the emitter body 403 described with reference to FIG. 4. The inner catheter body 3502 may be an example implementation of the inner shaft 114 described with reference to FIG. 1 and/or the inner shaft 414 described with reference to FIG. 4. The lumen 3504 may be sized to accommodate relatively large guidewires, including guidewires having diameters of between about 0.025 inch (0.635 mm) and about 0.095 inch (2.413 mm), approximately or equal to 0.035 inch (0.889 mm), or larger, as described with reference to FIGS. 1 and 15. The emitter body 3506 may be an example implementation of the emitter body 403 described with reference to FIG. 4 and/or the ring electrode member 1906 described with reference to FIGS. 19A-19D. The conductor 3508 may be an example implementation of the conductor 412 described with reference to FIG. 4 and/or the wire electrode member 1908 described with reference to FIGS. 19A-19D. The insulator 3510 may be an example implementation of the conductor insulation described with reference to the conductors 512 of FIG. 5. The adhesive 3512 may, for example, bond the emitter body 3506, the conductor 3508, and/or the insulation 3510 to each other and/or to the inner catheter body 3502. In some embodiments, the adhesive 3512 may include an ultraviolet adhesive with shadow curing properties, such as described with reference to embodiment 118. The encapsulating material 3514 may be an example implementation of the capsule 301 described with reference to FIG. 3 and/or the encapsulating dielectric material described with reference to FIGS. 19C and 27B.

In some embodiments, the encapsulating material 3514 may include parylene. In some embodiments the encapsulating material 3514 may, for example, include an epoxy. The epoxy may, for example, contain photo-setting and/or thermo-setting polymer. The epoxy may, for example, be one or more parts. The epoxy may, for example, be selected to bind to substrates of which the emitter component(s) and/or other catheter component(s) are made. As an illustrative example, the epoxy may include a diphenol resin. The epoxy may include a polyamine. The epoxy may, for example, include a carboxyl ester (e.g., epoxypropyl neodecanoate).

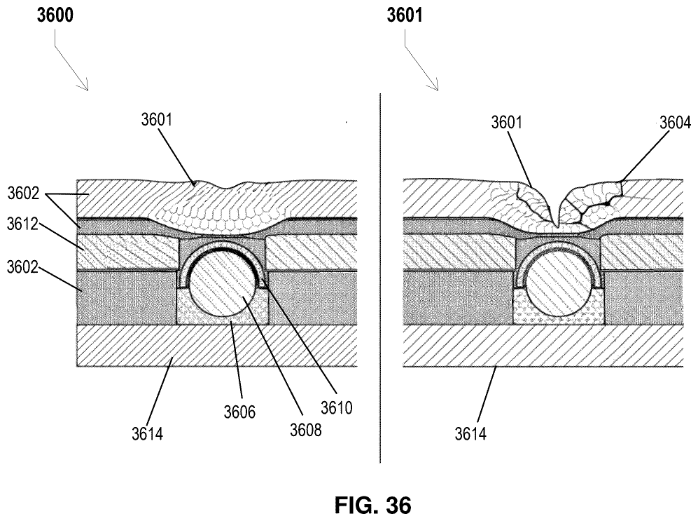

In some embodiments, the encapsulating material 3514 may include a first adhesive layer and a second potting layer disposed over the first adhesive layer, such as further described with reference to FIG. 36. In some embodiments, the first adhesive layer (e.g., adhesive 3512) may include a UV-curable tack adhesive. In some embodiments, the first adhesive layer may be selected for substrate compatibility with the emitter body material and the conductor insulation material. In some embodiments, the first adhesive layer may be applied prior to application of the second potting layer. In some embodiments, the first adhesive layer may, for example, promote adhesion between the emitter body 3506 and the second potting layer. In some embodiments, the first adhesive layer may alternatively or additionally be heat-cured. In some embodiments, the second potting layer may include a ductile epoxy, such as further described with reference to FIG. 36.

FIG. 36 depicts comparative cross-sections illustrating deformation behavior of two encapsulating material configurations during a spark generation event. The cross-section shows layers flat for ease of reference, irrespective of actual curvature. The emitter module may, for example, advantageously be used in an IVL system (e.g., peripheral, coronary). As shown in FIG. 36, a UV-cure tack adhesive first layer is disposed between the emitter body 3612 and the ductile epoxy potting second layer 3602. The ductile epoxy potting second layer 3602 is disposed over the UV-cure tack adhesive first layer and encapsulates the emitter body 3612 and a conductor 3608 (with conductor insulator 3610). The emitter body 3612 may be an example implementation of the ring electrode member 1906 described with reference to FIGS. 19A-19D. The conductor 3608 may be an example implementation of the wire electrode member 1908 described with reference to FIGS. 19A-19D.

As shown in a left panel of FIG. 36, a peripheral IVL configuration 3600 includes a ductile encapsulant (e.g., the ductile epoxy potting second layer 3602) disposed about a catheter inner body 3614. A deformation region 3601 of the ductile encapsulant illustrates deformation without brittle fracture during a spark generation event. As shown in a right panel of FIG. 36, a coronary IVL configuration includes a harder encapsulating material disposed about the catheter inner body 3614. Brittle fracture lines 3604 illustrate brittle fracture behavior of the harder encapsulating material during a spark generation event. The ductile deformation behavior illustrated by the deformation region 3601 of the peripheral IVL configuration 3600 may, for example, reduce particulate generation within the balloon interior volume during repeated discharge events relative to the brittle fracture behavior illustrated by the brittle fracture lines 3604 of the coronary IVL configuration. The comparative illustration illustrates an example functional distinction between the two material configurations and is not intended to limit the scope of the disclosure.

In some embodiments, the second potting layer (e.g., the ductile epoxy potting second layer 3602 described with reference to FIG. 36) may include a ductile epoxy. In some embodiments, the ductile epoxy may be selected based on one or more of the following material properties: post-cure elongation at break, substrate compatibility, processing temperature, and post-cure elasticity. In some embodiments, the post-cure elongation at break of the ductile epoxy may be at least 1%. In some embodiments, the post-cure elongation at break may be at least 2.5%. In some embodiments, the post-cure elongation at break may be less than 3%. Some embodiments may have a 3% or higher post-cure elongation at break. In some embodiments, a higher post-cure elongation at break may, for example, advantageously enable the encapsulating dielectric material to deform during a spark generation event without brittle fracture, such as illustrated in the left panel of FIG. 36. In some embodiments, such deformation behavior may, for example, reduce particulate generation within the balloon interior volume during repeated discharge events.

In some embodiments, the ductile epoxy may be selected to have a lower processing temperature than other encapsulating materials. In some embodiments, the processing temperature of the ductile epoxy may be less than 100 degrees Celsius. In some embodiments, the processing temperature may be less than 80 degrees Celsius. A lower processing temperature may, for example, reduce thermal exposure of adjacent structures including conductor insulation, balloon material, and inner shaft material during manufacturing.

In some embodiments, the encapsulating dielectric material may be selected based on compatibility with a substrate material of the emitter body and a substrate material of the discharge conductor. In some embodiments, the encapsulating dielectric material may be selected to adhere to stainless steel emitter body surfaces and copper conductor surfaces without delamination during repeated discharge events.

In some embodiments, encapsulating material may have elongation (e.g., at break) greater than 3%. For example, in some embodiments, elongation may be greater than 80% (e.g., post curing). For example, some embodiments may use flexible epoxies with elongation of 80%-150% or greater. Some embodiments may, for example, use epoxies with thermal post-curing properties and/or application processes. Post-curing may, for example, increase fracture toughness and/or ductility. Some embodiments may use an adhesive as an encapsulating material. An adhesive encapsulating material may have, for example, greater than 150% elongation. For example, some embodiments may have 300% or greater elongation (e.g., post-curing/setting). Increased elongation may, for example, advantageously reduce risk of fracturing and/or shedding of particles during operation in the vasculature.

In some examples, representative circuit parameters used to illustrate the curves discussed (e.g., with respect to FIGS. 32-34) may be consistent with voltage progression schedules empirically developed by the applicant for IVL emitter architectures, in which applied voltage was incrementally increased over successive discharge events, such as to compensate for electrode erosion and/or maintain consistent acoustic pressure output (e.g., as described with reference to FIGS. 16 and 18).

Although various embodiments are shown and described, other embodiments are contemplated.

In some embodiments, the apparatus and methods disclosed herein may be applied to treatment of calcified lesions in coronary vessels, including left anterior descending artery, left circumflex artery, and/or right coronary artery. In some embodiments, the apparatus may be configured for treatment of calcified lesions in renal arteries. In some embodiments, the apparatus may be configured for treatment of calcified lesions in mesenteric arteries. In some embodiments, the apparatus may be configured for treatment of calcified lesions in subclavian arteries. In some embodiments, the apparatus may be configured for treatment of calcified lesions in carotid arteries. In some embodiments, the apparatus may be configured for treatment of calcified lesions in popliteal arteries. In some embodiments, the apparatus may be configured for treatment of calcified lesions in tibial arteries. In some embodiments, the apparatus may be configured for treatment of calcified lesions in aortic and/or iliac vessels. In some embodiments, the apparatus may be configured for treatment of calcified lesions in venous vessels, including venous calcifications associated with chronic venous disease and/or venous stenting procedures.

In some embodiments, the apparatus and methods disclosed herein may be applied to treatment of calcified lesions in superficial femoral artery (SFA) and/or common femoral artery (CFA) vessels. In some embodiments, the apparatus may be configured for treatment of calcified lesions in iliac arteries, including common iliac and/or external iliac arteries. In some embodiments, the apparatus may be configured for treatment of calcified lesions in below-the-knee vessels, including anterior tibial, posterior tibial, and/or peroneal arteries. In some embodiments, the apparatus may be configured for treatment of calcified lesions in the profunda femoris artery. In some embodiments, the apparatus may be configured for treatment of calcified lesions in the popliteal artery, including above-knee and below-knee popliteal segments.

In some embodiments, the apparatus may be used for preparation of calcified landing zones prior to endovascular aortic repair (EVAR) and/or thoracic endovascular aortic repair (TEVAR). In some embodiments, the apparatus may be used for preparation of calcified vessel segments prior to delivery of self-expanding and/or balloon-expandable stents. In some embodiments, the apparatus may be used for preparation of calcified vessel segments prior to delivery of stent grafts. In some embodiments, the apparatus may be used for preparation of calcified vessel segments prior to delivery of atherectomy devices. In some embodiments, the apparatus may be used for preparation of calcified vessel segments prior to drug-coated balloon delivery, such as to increase drug uptake in calcified tissue. In some embodiments, the apparatus may be used for preparation of calcified annular tissue prior to transcatheter aortic valve replacement (TAVR) and/or transcatheter mitral valve replacement (TMVR). In some embodiments, the apparatus may be used for preparation of calcified tissue prior to percutaneous transluminal angioplasty (PTA). In some embodiments, the apparatus may be used in combination with rotational atherectomy, orbital atherectomy, and/or laser atherectomy as part of a multi-modality vessel preparation strategy.

In some embodiments, the apparatus may be configured for neurovascular applications, such as for treatment of calcified intracranial and/or extracranial vessel segments. In some embodiments, the apparatus may be configured for treatment of calcified carotid bifurcation lesions prior to carotid artery stenting. In some embodiments, the apparatus may be configured for treatment of calcified intracranial atherosclerotic disease prior to intracranial stenting. In some embodiments, the compact radial packaging geometry and large guidewire lumen compatibility disclosed herein may, for example, be particularly advantageous for neurovascular applications in which vessel tortuosity and small vessel diameters impose significant constraints on catheter profile and trackability.

In some embodiments, the apparatus may be combined with one or more additional energy delivery modalities within a single catheter platform. In some embodiments, the apparatus may be combined with radiofrequency (RF) ablation capability, such as for simultaneous and/or sequential treatment of calcified and/or fibrotic tissue. In some embodiments, the apparatus may be combined with cryotherapy capability, such as for simultaneous and/or sequential treatment of calcified and/or inflammatory tissue. In some embodiments, the apparatus may be combined with laser energy delivery capability, such as for simultaneous and/or sequential treatment of calcified and/or thrombotic tissue. In some embodiments, the apparatus may be combined with ultrasound energy delivery capability, such as for simultaneous and/or sequential treatment of calcified tissue and/or for imaging guidance. In some embodiments, the apparatus may be combined with drug delivery capability, such as for simultaneous and/or sequential delivery of anti-restenotic, anti-inflammatory, and/or anti-thrombotic agents to a treatment site. In some embodiments, the PSSE emitter architecture may be configured to operate as a component of a multi-energy catheter platform in which the compact radial packaging geometry facilitates integration of multiple energy delivery systems within a single catheter profile.

In some embodiments, the apparatus may further include one or more sensors configured to measure one or more parameters associated with electrical discharge events and/or tissue response. In some embodiments, the sensors may include pressure sensors configured to measure pressure wave magnitude and/or propagation characteristics within the balloon. In some embodiments, the sensors may include impedance sensors configured to measure electrical impedance of tissue adjacent to the emitter modules, such as to assess calcification severity and/or treatment progress. In some embodiments, the sensors may include temperature sensors configured to measure temperature within the balloon and/or adjacent tissue during lithotripsy therapy. In some embodiments, sensor data may be used to adjust one or more parameters of the power profile, such as applied voltage, pulse count, and/or pulse repetition rate, in real time and/or according to a predetermined feedback algorithm. In some embodiments, sensor data may be used to verify correct positioning of the emitter modules relative to a target lesion prior to initiating therapy. In some embodiments, the apparatus may include one or more imaging elements, such as intravascular ultrasound (IVUS) transducers and/or optical coherence tomography (OCT) elements, configured to provide real-time imaging of the target lesion and/or treatment zone during lithotripsy therapy.

In some embodiments, the apparatus may be configured for compatibility with peripheral access systems, including less than 6 French, 6 French, 7 French, 8 French, and/or larger than 8 French introducer sheaths. In some embodiments, the apparatus may be configured for compatibility with long introducer sheaths used in peripheral and/or complex endovascular procedures. In some embodiments, the apparatus may be configured for compatibility with steerable guide catheters and/or guide sheaths used in structural heart and/or neurovascular procedures. In some embodiments, the apparatus may be configured for compatibility with rapid exchange monorail guidewire systems. In some embodiments, the apparatus may be configured for compatibility with over-the-wire guidewire systems. In some embodiments, the apparatus may be configured for use without a guidewire in applications where a guidewire lumen is not indicated, such as in direct puncture and/or open surgical applications. In some embodiments, the guidewire lumen may be repurposed as an infusion lumen for delivery of contrast media, saline, and/or therapeutic agents when a guidewire is not in use.

In some embodiments, the guidewire lumen may be configured as an over-the-wire guidewire lumen. In some embodiments, the guidewire lumen may be configured as a rapid exchange guidewire lumen. In some embodiments, the guidewire lumen may be sized to receive a guidewire having a diameter of between about 0.025 inch (0.635 mm) and about 0.095 inch (2.413 mm), including equal to or about 0.035 inches (0.889 mm). In some embodiments, the guidewire lumen may be sized to receive a guidewire having a diameter of at least 0.018 inches (0.457 mm). In some embodiments, the guidewire lumen may be sized to receive a guidewire having a diameter of at least 0.014 inches (0.356 mm). In some embodiments, the catheter may be configured with a crossing profile less than or equal to 0.085 inches (2.159 mm), such as to fit within a 7 French or smaller introducer sheath. In some embodiments, the crossing profile may be less than or equal to 0.065 inches (1.651 mm), such as to fit within a 6 French or smaller introducer sheath. In some embodiments, the crossing profile may be less than or equal to 0.061 inches (1.549 mm), such as to fit within a 5 French or smaller introducer sheath. In some embodiments, the catheter may be as small as a 4-5 French catheter while still accommodating guidewire lumen dimensions configured to receive a 0.035-inch (0.889 mm) guidewire.

In some embodiments, emitter modules may include two emitter modules. In some embodiments, the emitter modules may include four emitter modules. In some embodiments, the emitter modules may include six emitter modules. In some embodiments, the emitter modules may include eight or more emitter modules. In some embodiments, the emitter modules may be disposed along a longitudinally extending shaft of the catheter body. In some embodiments, adjacent emitter modules of the array of emitter modules may be electrically connected by bridge conductors. Each bridge conductor may be configured to conduct current from a first emitter module to a second emitter module upon occurrence of an electrical arc across a spark gap of the first emitter module. In some embodiments, the at least one floating bridge wire may be shorter in length by a factor of at least 100 than one or more conductors electrically connected to an electrical source prior to the electrical arc.

In some embodiments, a width of the discharge aperture may be between 1.0 and 3.0 times a diameter of the discharge conductor. In some embodiments, a depth of the discharge aperture may be between 0.3 and 0.7 times a wall thickness of the emitter body. In some embodiments, the discharge aperture may include longitudinally extending symmetric surfaces on either side of a plane passing through the longitudinal centerline and through a center of the discharge aperture. In some embodiments, each of at least two of the exposed discharge surfaces may be defined by a region of the discharge aperture from which an encapsulating dielectric material is absent. In some embodiments, the region of the discharge aperture from which the encapsulating dielectric material is absent may be formed by removal of a portion of the encapsulating dielectric material after application of the encapsulating dielectric material over the emitter body and the discharge conductor.

In some embodiments, a shaped internal surface of the conductor channel may mechanically retain the discharge conductor without adhesive bonding. In some embodiments, the shaped internal surface may retain the discharge conductor within the conductor channel, such as even without an external support member. In some embodiments, a conductor channel may extend both radially inward from an outermost radial surface of the emitter body and longitudinally inward from at least one end of the emitter body such that the conductor channel defines both a path of at least one of the conductors and a predetermined spark progression path. In some embodiments, a circumferential width of the conductor channel may be narrower than a diameter of the at least one conductor in a conductor retention region of the conductor channel, distal to an initial spark gap location, such that successive spark gaps as the at least one conductor erodes along the predetermined spark progression path may have a first edge-to-edge distance greater than when the discharge conductor erodes into the conductor retention region.

In some embodiments, an outermost diameter of the discharge conductor may be equal to a thickness of the emitter body measured between an outer surface and an inner surface of the emitter body. In some embodiments, the discharge conductor may be positioned sub-flush relative to an outer surface of the emitter body such that no portion of the discharge conductor protrudes beyond the outer surface of the emitter body. In some embodiments, the discharge conductor, the first exposed discharge surface, and the second exposed discharge surface may all be positioned within a volumetric shell bounded by the outer surface of the emitter body and the inner surface of the emitter body. In some embodiments, the diameter of the wire electrode member may be at least 80% of the thickness of the ring electrode member. In some embodiments, the diameter may be at least 85% of the thickness. In some embodiments, the diameter may be at least 90% of the thickness. In some embodiments, the diameter may be at least 95% of the thickness. A relationship closer to 1:1 may, for example, enable a thinner radial profile.

In some embodiments, the ratio of the surface area of the first exposed discharge surface to the surface area of the second exposed discharge surface may be less than or equal to 10:1 after a final discharge event of multiple discharge events. In some embodiments, the ratio of the surface areas may vary over multiple discharge events as electrode material erodes during repeated spark formation cycles. In some embodiments, at least one of the exposed discharge surfaces may include multiple exposed discharge surfaces positioned on opposing sides of a discharge conductor within a discharge aperture. In some embodiments, the total exposed emitter body surface area may be made up of one or more exposed emitter body surface areas, totaling a first area. The total exposed wire conductor surface area may be made up of one or more exposed wire conductor surface areas, totaling a second area. In some example embodiments, the ratio of the first area to the second area may be 1:1 or substantially 1:1.

In some embodiments, at least one of the exposed discharge surfaces may be defined by a cavity in a dielectric material otherwise encapsulating the emitter module and electrically isolating from each other conductive surfaces of the emitter module. In some embodiments, the exposed discharge surfaces may be contained within a spark initiation volumetric shell concentric with the longitudinal centerline. The spark initiation volumetric shell may have an inner radius defined by a lowermost first exposed discharge surface. The spark initiation volumetric shell may have an outer radius defined by an outermost first exposed discharge surface. In some embodiments, the emitter module may include a body with an interrupted annular shaped cross section extending at least partially circumferentially about the longitudinal centerline. In some embodiments, the first exposed discharge surface and the second exposed discharge surface may each be positioned at a common spark initiation radius relative to the longitudinal centerline, such that an arcuate surface at the common spark initiation radius passes through both the first exposed discharge surface and the second exposed discharge surface. In some embodiments, the inner radius of the spark initiation volumetric shell may be greater than a distance from the longitudinal centerline to an inner surface of the emitter body. The emitter body may, for example, have rounded and/or blunt ends. The rounded/blunted ends (e.g., such as toroidal/semi-toroidal shape) may advantageously avoid inadvertent balloon damage, among other advantages.

In some embodiments, a first edge-to-edge distance between a pair of the exposed discharge surfaces may be less than a median electrode distance between the pair. In some embodiments, a first edge-to-edge distance between a pair of the exposed discharge surfaces may decrease along the longitudinal centerline progressing away from a terminal end of the discharge conductor. In some embodiments, the first edge-to-edge distance may be defined by a diameter of a discharge conductor, an inner diameter of an emitter body, and a radial depth of insulation termination. In some embodiments, a ratio of the median electrode distance to the first edge-to-edge distance may be greater than 1:1. In some embodiments, a first edge-to-edge distance between a pair of the exposed discharge surfaces may be constant or substantially constant along an erosion path of a discharge conductor during repeated spark formation cycles. In some embodiments, a first edge-to-edge distance between a pair of the exposed discharge surfaces at a first spark initiation site may be less than a second edge-to-edge distance between the exposed discharge surfaces at a laterally offset spark initiation site, such that electrical discharge initiates preferentially at the first spark initiation site prior to initiating at the laterally offset spark initiation site.

In some embodiments, the channel width may be less than one-half of a circumferential extent of the first electrode member. In some embodiments, reduction of the channel width may increase discharge current confinement without reducing the first edge-to-edge distance. In some embodiments, reduction of the first edge-to-edge distance may reduce the breakdown voltage without increasing the channel width. In some embodiments, the channel width may be independent of a radial depth of insulation removal that defines a first edge-to-edge distance between a pair of exposed discharge surfaces. In some embodiments, the first edge-to-edge distance may be defined by: (i) a diameter of the second electrode member, (ii) an inner diameter of the first electrode member, (iii) a radial depth of insulation termination, and (iv) a width of a channel formed in the first electrode member. In some embodiments, the channel width may constrain a lateral dimension of plasma expansion during discharge independently of a spacing between the first electrode member and the second electrode member. In some embodiments, the first electrode member may include a ring electrode member. The second electrode member may include a wire electrode member disposed within a conductor channel of the ring electrode member.

In some embodiments, the non-linear path may include a continuous curvature in the circumferential direction. In some embodiments, the non-linear path may include one or more inflection regions along the conductor channel. In some embodiments, the conductor channel may follow a non-linear path in at least two dimensions. In some embodiments, the conductor channel may vary in cross-sectional width along the non-linear path. In some embodiments, the non-linear path may define a varying edge-to-edge distance between the discharge conductor and the opposing surfaces of the emitter body at multiple locations along the conductor channel. In some embodiments, spark initiation may migrate along the non-linear path during progressive electrode erosion, distributing erosion along the conductor channel. In some embodiments, curvilinear may refer to a linear path non-parallel to the longitudinal axis.

In some embodiments, the conductor channel may define a spark progression path along which spark initiation locations migrate during progressive electrode erosion. In some embodiments, an encapsulating dielectric material may bound the first exposed discharge surface and the second exposed discharge surface, wherein electrical discharge along insulated portions of the emitter body is inhibited such that the discharge conductor guides erosion along a predetermined spark progression path. In some embodiments, a first edge-to-edge distance between the second exposed discharge surface and the first exposed discharge surface may vary over multiple discharge events as electrode material erodes along the spark progression path. In some embodiments, a breakdown voltage initiating electrical discharge may change monotonically as spark initiation locations migrate along the spark progression path. In some embodiments, the spark progression path may follow a non-linear path of the conductor channel with spatial variation in edge-to-edge distances along the spark progression path. In some embodiments, the apparatus may further include a voltage controller configured such that an applied voltage is adjusted over multiple spark events according to a predetermined progression schedule corresponding to an expected spark gap size at a present position along the spark progression path.

In some embodiments, the apparatus may further include a degassing membrane disposed about the catheter body. In some embodiments, the degassing membrane may include a semi-permeable polymer membrane. In some embodiments, conductors connecting the emitter module to an electrical source may be disposed along an outer surface of a shaft of the catheter body. In some embodiments, the emitter body may be bonded to a shaft of the catheter body by an adhesive including an ultraviolet adhesive with shadow curing properties. In some embodiments, the emitter body may include multiple conductor channels. Each conductor channel may house a respective discharge conductor. In some embodiments, the emitter body may include multiple discharge apertures and at least a corresponding number of discharge conductors.

In some embodiments, one or more conductors of the catheter may be communicably coupled to a power source, such as a voltage source and/or pulse generator. The power source may, for example, provide power, such as in the form of pulses, to generate sparks at one or more emitter modules. In some embodiments, the power source may be communicably coupled to a control module. The control module may, for example, be operated automatically and/or manually to operate the power source. In some embodiments, the control module may be integrated in the power source. In some embodiments, the control module may be configured to apply a predetermined voltage schedule corresponding to expected geometric changes along a spark progression path of one or more emitter modules, such as described with reference to FIG. 16.

In some embodiments, a working length of the IVL assembly may be 1000 mm or greater. In some embodiments, the working length may be 1100 mm or greater. In some embodiments, the working length may be 1200 mm or greater. In some embodiments, the working length may be 1300 mm or greater. In some embodiments, the working length may be 1400 mm or greater. Such working lengths may, for example, be particularly suitable for peripheral vascular applications involving longer calcified segments and/or larger vessels. Working length in this context may refer to a usable length of a catheter shaft measured from the proximal hub to the distal tip. For example, working length may exclude catheter portion(s) not intended to be introduced into the patient. Working length may, for example, not include the length of any external connector, handle, and/or proximal interaction module.

In some embodiments, each emitter module may be defined by a separate transfer function that defines a voltage response and/or spark formation response to a power input. In some embodiments, emitter module transfer functions may be customized within a single IVL assembly. In some embodiments, a single applied input, such as a high-voltage pulse from a control module, may have a specific rise time when initially introduced into the system. As this voltage encounters different spark locations, the mechanical design of each circuit element in series may modify this rise time to a distinct value characteristic of that location and/or group of locations. Such alteration may, for example, be achieved by adjustment and/or selection of localized circuit components such as specific conductor configurations, emitter designs, and/or the use of different dielectric materials. In some embodiments, bridge conductors may experience more mechanical damage than other conductors. Without being bound to a particular theory, bridge conductors may have a shorter rise time, as the voltage may be suddenly applied as a spark is formed. Some embodiments may, for example, be configured to approximate the voltage pulse by decreasing rise time of the source voltage and/or shortening wires. Some embodiments may, for example, increase wire gauge of bridge conductors, which may, for example, increase resistance to mechanical damage.

In some embodiments, the emitter body may include one or more cutouts. Cutouts may, for example, allow wire routing to and/or from distal emitters to be flush with proximal emitters. Such embodiments may, for example, reduce a crossing profile of the IVL assembly. In some embodiments, a wire configuration including bridge wires and/or conductor channels such that conductors are flush with the outer periphery of the emitter body may enable increased emitter body wall thickness. Increased wall thickness may, for example, increase the emitter body's resistance to erosion during repeated spark generation events, potentially increasing durability and/or longevity of the emitter body over multiple emission events.

In some embodiments, a single return conductor may be used. In some embodiments, fewer return conductors may be used than supply conductors. In some embodiments, an equal or greater number of return conductors may be used than supply conductors. In some embodiments, the return conductor may also be referred to as a ground conductor.

In some embodiments, the encapsulating dielectric material may include a photopolymerized adhesive with shadow curing properties. In some embodiments, the encapsulating dielectric material may be applied after the emitter body and the discharge conductor are assembled to a shaft of the catheter body. In some embodiments, the encapsulating dielectric material may be formed by coating, potting, over-molding, and/or combinations thereof. In some embodiments, the encapsulating dielectric material may provide mechanical support and/or positional stability for conductive elements relative to the inner shaft.

In some embodiments, conductive material deposited between the emitter body surface and the conductor surface during repeated discharge events may be guided along a predetermined spark progression path by the bounding geometry of the encapsulating dielectric material, such that refuse accumulation, such as carbon accumulation, does not cause a fixed short-circuit between the emitter body and the conductor at a single location.

In some embodiments, the termination of the ring electrode insulative member and/or the wire electrode insulative member defining the spark initiation locations may be generated by a subtractive operation, such as cutting a notch. In some embodiments, the subtractive operation may include laser ablation, mechanical cutting, and/or chemical etching.

In some IVL emitter configurations, a stationary central conductor, such as a thin wire, may be positioned within a surrounding ring electrode, with spark initiation occurring at a fixed location between the two electrodes. Because the spark initiation location in such configurations does not change, repeated discharge events may concentrate erosion at a single site on the conductor and/or the ring electrode. Such concentrated erosion may progressively alter the effective spark gap distance at the fixed location, potentially causing the breakdown voltage to change unpredictably over the operational life of the emitter. Additionally, concentrated erosion at a single site may cause conductive material, such as a slag bridge, to accumulate between the conductor and the ring electrode at the fixed location, potentially creating a conductive path that short-circuits the spark gap and/or renders the emitter inoperative. In contrast, the PSSE architecture disclosed herein may bound the exposed discharge surfaces with an encapsulating dielectric material, such as the capsule 301 described with reference to FIG. 3 and/or the ring electrode insulative member 1910 and the wire electrode insulative member 1912 described with reference to FIGS. 19C and 19D, such that spark initiation locations migrate along a predetermined spark progression path as electrode material erodes during successive discharge events. For example, the spark initiation location may migrate from the ring electrode channel window 3002 into the ring electrode channel 3004 described with reference to FIGS. 30A and 30B. Because the bounding geometry of the encapsulating dielectric material guides the spark initiation location along the predetermined spark progression path, conductive material deposited between the emitter body surface and the conductor surface during repeated discharge events, such as a slag bridge, may be guided along the predetermined spark progression path rather than accumulating at a single fixed location, as described with reference to FIG. 3. Such guided migration may, for example, distribute erosion over a larger electrode surface area and/or reduce the likelihood that refuse accumulation causes a fixed short-circuit between the emitter body and the conductor at a single location.

The encapsulating dielectric material may, for example, physically bound the exposed discharge surfaces such that spark initiation locations are constrained to migrate along a predetermined spark progression path defined by the geometry of the conductor channel. This bounding may, for example, prevent spark initiation locations from migrating stochastically across the electrode surfaces (e.g., such that erosion progresses deterministically along the predetermined spark progression path rather than at random sites).

The geometric basis for gap preservation along the spark progression path may be understood with reference to the mathematical relationship described with reference to FIGS. 23A and 23B. As described therein, the initial spark generation first edge-to-edge distance 2312 at a given spark initiation location may be approximated as

$${d}_{1} = \frac{{ID}_{ring} - {d}_{wire}}{2} - {t}_{insulationremoval} ,$$

where ID_{ring} is the inner diameter of the ring electrode member 1930 described with reference to FIG. 19C, d_{wire} is the diameter of the wire electrode member 1926 described with reference to FIG. 19C, and tᵢₙₛᵤₗₐₜᵢₒₙᵣₑₘₒᵥₐₗ is the initial spark depth of insulation removal distance 2310 described with reference to FIGS. 23A and 23B. As the wire electrode member 1908 erodes along the spark progression path, the first edge-to-edge distance d₁ at each successive spark initiation location is determined by this same geometric relationship, evaluated at the longitudinal position of the current spark initiation location, rather than by the cumulative erosion history at a single fixed point. Because the conductor channel geometry, such as the ring electrode channel 3004 described with reference to FIGS. 30A and 30B, defines the spatial envelope within which the wire electrode member 1908 erodes, and because the encapsulating dielectric material bounds the exposed discharge surfaces along the spark progression path, the geometric parameters that determine d₁, including the inner diameter of the ring electrode member 1906, the diameter of the wire electrode member 1908 at that location, and the depth of insulation removal, are structurally defined at each position along the path. The first edge-to-edge distance d₁ at each successive spark initiation location is thus a function of the local channel geometry at that position rather than a function of cumulative material loss at a single location, which may, for example, facilitate a more controlled and/or predictable breakdown voltage across successive discharge events relative to fixed-electrode configurations in which the effective spark gap distance is progressively altered by concentrated erosion.

The channel width, such as the channel width 2504 described with reference to FIG. 25 and/or the channel width variation between the ring electrode channel window 3002 and the ring electrode channel 3004 described with reference to FIGS. 30A and 30B, may determine the magnitude of discharge current following breakdown independently of the first edge-to-edge distance d₁. As described, such as with reference to FIGS. 30A and 30B, the discharge current I following electrical breakdown may be approximated as proportional to the channel width W_{channel}, such that I oC W_{channel}. Because the channel width W_{channel} and the first edge-to-edge distance d₁ are each determined by independent geometric parameters, the breakdown voltage and the discharge current magnitude may each be independently configured by respective geometric features of the emitter module, such as, for example, described with reference to FIGS. 23A, 23B, 30A, and 30B. The curvilinear boundary between the ring electrode channel window 3002 and the ring electrode channel 3004 may, for example, encode a progressive variation in W_{channel} along the spark progression path, such that the discharge current magnitude varies predictably as spark initiation locations migrate along the path during progressive electrode erosion. Such geometric encoding of both breakdown voltage and discharge current magnitude may, for example, allow the emitter module to be operated within a limited generator voltage range across a large number of discharge cycles, such as by applying a predetermined voltage schedule corresponding to expected geometric changes along the spark progression path, such as described with reference to the emission cycle plot 1600 of FIG. 16 and/or the power profile selection at step 1808 of the method 1800 described with reference to FIG. 18.

In some embodiments, the operational life of the emitter module may be characterized by multiple behavioral states corresponding to different positions of the spark initiation location along the spark progression path. As an illustrative example, three states may be included. In a first state, spark initiation occurs at or near the initial spark generation region, such as the ring electrode channel window 3002 described with reference to FIGS. 30A and 30B, where the channel width and the first edge-to-edge distance are at their initial design values. In a second state, spark initiation has migrated along the spark progression path into the ring electrode channel 3004, where the channel width and/or the first edge-to-edge distance may differ from their initial values due to the geometry of the conductor channel at the current spark initiation location. In a third state, the discharge conductor has eroded to the distal end of the conductor channel, and the emitter module may be at or near the end of its operational life. In some embodiments, the voltage progression schedule applied by the control module may be configured to correspond to the expected geometric changes at each of these three states, such that consistent acoustic pressure output is maintained across the full operational life of the emitter module.

In some embodiments, gas accumulation within the balloon interior volume from prior discharge events may attenuate acoustic pressure wave propagation efficiency, such as by acting as a compressible medium that absorbs a portion of the pressure wave energy generated by each discharge event. Such attenuation may, for example, be a factor in selecting a voltage progression schedule that increases applied voltage over successive discharge events to maintain consistent acoustic pressure output at the vessel wall. In some embodiments, degassing of the balloon, such as by deflating and re-inflating the balloon as described with reference to step 1816 of the method 1800, may reduce accumulated gas volume and restore acoustic pressure propagation efficiency.

In some embodiments, a rest period between successive discharge pulses may be selected to restrain cumulative temperature rise within the balloon fluid. For example, a rest period may be selected to maintain the temperature rise below a predetermined threshold (e.g., less than approximately 2 degrees Celsius above baseline). In some embodiments, the balloon 106 of the catheter 104 may serve a dual purpose during a treatment procedure, functioning both as a fluid-filled environment for lithotripsy pressure wave generation and as a dilation balloon for mechanical expansion of a treated vessel segment. In such embodiments, the balloon 106 may first be inflated to a first pressure sufficient to contact a vessel wall and establish a fluid medium surrounding the emitter modules 116, such as described with reference to step 1804 of the method 1800 of FIG. 18. Electrical discharges may then be generated by the emitter modules 116 in accordance with the method 1800, such as by operating the power source 1560 and/or the control module 1562 to apply voltage pulses that produce pressure waves propagating through the fluid-filled balloon 106 and into adjacent calcified tissue. After a target number N of pulses has been applied, such as determined at decision step 1814 of the method 1800, the balloon 106 may subsequently be inflated to a second pressure higher than the first pressure through the balloon interface port 1552 of the proximal interaction module 1502, such as to mechanically dilate the treated vessel segment. The second pressure may, for example, be selected to achieve a target vessel lumen diameter at the treatment site. In some embodiments, the sequential application of lithotripsy pressure waves followed by balloon dilation using the same balloon 106 may reduce or avoid device exchange between lithotripsy treatment and balloon angioplasty, which may, for example, reduce procedural time, reduce fluoroscopy exposure, and/or reduce risk of losing guidewire position during a device exchange.

In some embodiments, the pressure waves generated by the emitter modules 116 during the lithotripsy phase may fracture and/or otherwise modify calcium deposits within the vessel wall, which may reduce the mechanical resistance of the calcified lesion to subsequent balloon dilation. Such reduction in mechanical resistance may, for example, allow the balloon 106 to achieve a target lumen diameter at a lower inflation pressure than would be required by balloon dilation alone in the absence of prior lithotripsy treatment. In some embodiments, the reduced inflation pressure associated with post-lithotripsy dilation may, for example, reduce risk of vessel dissection, perforation, and/or barotrauma relative to high-pressure balloon dilation of an untreated calcified lesion. In some embodiments, the dual-purpose use of the balloon 106 may be performed as part of a percutaneous transluminal angioplasty (PTA) procedure, in which the lithotripsy treatment and the balloon dilation are performed sequentially using the catheter 104 without withdrawing the catheter 104 from the treatment site between the lithotripsy phase and the dilation phase.

In some embodiments, the balloon 106 may be deflated and re-inflated one or more times between the lithotripsy phase and the dilation phase, such as to degas the balloon 106 by expelling gas accumulated during the lithotripsy phase, as described with reference to step 1816 of the method 1800 of FIG. 18. Degassing prior to the dilation phase may, for example, allow the balloon 106 to be inflated with a liquid medium or substantially liquid medium during the dilation phase, which may, for example, provide more uniform radial force distribution during mechanical dilation relative to a balloon containing residual gas pockets. In some embodiments, the dilation phase may be repeated at one or more additional longitudinal positions along the vessel by repositioning the catheter 104 along a guidewire extending through the guidewire lumen, such as without withdrawing the catheter 104 from the vessel.

In some embodiments, the emitter module may be configured such that a spark gap defined between the first exposed discharge surface and the second exposed discharge surface is oriented at a predetermined angular position relative to a circumferential reference of the catheter. The angular position of the spark gap may, for example, be selected to direct a higher-magnitude pressure wave toward a target tissue region, such as a calcified vessel wall segment.

Without being bound to a particular theory, electrical discharge across the spark gap may generate a pressure wave having a non-uniform circumferential distribution, with a higher pressure magnitude at an angular position corresponding to the spark gap location relative to the longitudinal centerline, and a lower pressure magnitude at an angular position diametrically opposed to the spark gap location. This directional pressure asymmetry may, for example, result from the localized nature of spark initiation at the defined exposed discharge surfaces and the geometry of plasma expansion within the fluid-filled balloon.

In some embodiments, the catheter may include one or more orientation indicators, such as radiopaque markers, echogenic markers, and/or surface features, configured to indicate the angular position of the spark gap relative to the catheter body under fluoroscopic and/or ultrasound imaging. Such orientation indicators may, for example, allow a physician to rotationally align the catheter such that the spark gap faces a target calcified tissue region prior to initiating lithotripsy therapy.

In some embodiments, the angular position of the spark gap may be characterized by measuring pressure wave magnitude at multiple angular positions relative to the spark gap, including at 0°, 90°, 180°, and 270° relative to the spark gap centerline. Such characterization may, for example, confirm that pressure wave magnitude is highest at the 0° position corresponding to the spark gap location and lowest at the 180° position diametrically opposed to the spark gap location. The ratio of pressure wave magnitude at 0° to pressure wave magnitude at 180° may, for example, be greater than 1.1:1, greater than 1.2:1, or greater than 1.5:1 in some embodiments.

In some embodiments, the emitter module may include multiple spark gaps arranged at predetermined angular positions about the circumference of the emitter body, such as to provide a target circumferential pressure distribution. For example, two spark gaps positioned at 0° and 180° may, for example, provide a bi-directional pressure distribution. Four spark gaps positioned at 0°, 90°, 180°, and 270° may, for example, provide a more uniform circumferential pressure distribution. The angular spacing and number of spark gaps may, for example, be selected based on a target pressure uniformity index, defined as a ratio of minimum to maximum pressure wave magnitude measured circumferentially about the emitter module.

In some embodiments, the angular orientation of the spark gap may be fixed relative to a rotational reference feature of the catheter, such as a handle feature, a guidewire port orientation, and/or a radiopaque marker, such that, for example, the physician can determine the spark gap orientation from outside the patient without, for example, direct visualization of the emitter module.

In some embodiments (e.g., including embodiments in which multiple PSSE modules are electrically coupled in series) spark-gap distances (e.g., of respective PSSE modules) may be selected (e.g., independently). The various spark-gap distances of the modules may be selected, by way of example and not limitation, based on module position, local conductor geometry, local impedance, dielectric geometry, and/or desired acoustic output. In some embodiments, at least two PSSE modules (e.g., in a series-coupled group) may have different spark-gap distances selected to tune breakdown behavior, acoustic output, and/or other parameter(s) across the group.

The control, sensing, feedback, and imaging functions described herein, including operation of the control module 1562 and the power source 1560 described with reference to FIG. 15, application of predetermined voltage schedules corresponding to expected geometric changes along a spark progression path, processing of sensor data for real-time adjustment of power profile parameters, and acquisition and processing of imaging data (e.g., from intravascular ultrasound (IVUS) transducers and/or optical coherence tomography (OCT) elements), may be implemented using various computing hardware, processor architectures, software platforms, interface technologies, power supply configurations, communication protocols, and/or device architectures. The communicable coupling between the power source 1560 and the control module 1562, the automated and/or manual operation of the power source 1560, the delivery of high-voltage pulses and/or control signals through the electrical cable 1554 and/or the electrical connector 1556 of the proximal interaction module 1502 described with reference to FIG. 15, may similarly involve various processor types, memory configurations, input/output interfaces, and/or wired and/or wireless communication technologies.

In some embodiments, the emitter body may be formed by additive manufacturing, such as metal additive manufacturing, by way of example and not limitation. In some embodiments, the metal additive manufacturing process may include, for example, a Resin Infused Powder Lithography (RIPL) process. Such a process may, for example, enable production of features having dimensions on the order of 0.001 inches (25.4 micrometers) or less. In some embodiments, the emitter body may be formed from a biocompatible metal alloy. The metal alloy may, for example, include stainless steel. The metal alloy may, for example, include a titanium alloy. The metal alloy may, for example, include a cobalt-chromium alloy.

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

Any ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, or “substantially” as used herein include the recited numbers (e.g., about 10% includes 10%), and also represent an amount close to the stated amount that still performs a desired function or achieves a desired result. For example, the terms “approximately”, “about”, and “substantially” may refer to an amount that is within 10% of, within 5% of, within 1% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

Following are some further example embodiments. These are presented only by way of example and are not intended to limit the scope of the disclosure in any way. Further, any example embodiment can be combined with one or more of the example embodiments.

Embodiment 1. An intravascular lithotripsy system, including: a catheter extending along a longitudinal axis, defining a guidewire lumen configured to receive a 0.025-inch (0.635 mm) to 0.095-inch (2.413 mm) guidewire; an inner shaft disposed within the catheter; and at least four emitter modules disposed along the inner shaft, each emitter module including: an emitter body having an emitter body surface extending along the longitudinal axis; at least one conductor having a conductor surface spaced from the emitter body surface by a spark gap; and a dielectric material insulating the at least one conductor from the emitter body surface except at a discharge location.

Embodiment 2. The intravascular lithotripsy system of embodiment 1, wherein the emitter body surface is disposed at a first radius from the longitudinal axis and the conductor surface is disposed at a second radius from the longitudinal axis and wherein the first radius is equal or substantially equal to the second radius such that a spark gap distance between the emitter body surface and the conductor surface remains constant or substantially constant as the conductor surface erodes during repeated spark generation events.

Embodiment 3. The intravascular lithotripsy system of embodiment 1 or 2, wherein the discharge location includes a window formed in the emitter body that exposes the emitter body surface and the conductor surface across the spark gap.

Embodiment 4. The intravascular lithotripsy system of any one of embodiments 1-3, wherein the at least one conductor is routed through a channel formed in the emitter body and is partially captured by the emitter body such that the conductor and the emitter body are inscribed within a common radial envelope.

Embodiment 5. The intravascular lithotripsy system of any one of embodiments 1-4, wherein each emitter module includes two spark gaps positioned circumferentially about the emitter body.

Embodiment 6. The intravascular lithotripsy system of any one of embodiments 1-5, wherein six emitter modules are arranged as three emitter pairs disposed within a balloon having a length of 60 mm or approximately 60 mm.

Embodiment 7. The intravascular lithotripsy system of embodiment 6, wherein each emitter pair is electrically isolated from other emitter pairs and is associated with a dedicated supply conductor and a dedicated return conductor.

Embodiment 8. The intravascular lithotripsy system of embodiment 6 or 7, wherein the guidewire lumen, the inner shaft, and the six emitter modules are dimensioned such that the catheter is introducible through a 7 French introducer sheath.

Embodiment 9. The intravascular lithotripsy system of any one of embodiments 1-8, wherein the guide-wire is a 0.025-inch (0.635 mm) to 0.038-inch (0.965 mm) guidewire.

Embodiment 10. The intravascular lithotripsy system of any one of embodiments 1-9, wherein the guidewire is an 0.035-inch (0.889 mm) guidewire.

Embodiment 11. An intravascular lithotripsy balloon catheter including: a catheter shaft extending between a proximal end and a distal end and defining a guidewire lumen sized to receive a 0.025-inch (0.635 mm) to 0.095-inch (2.413 mm) guidewire; an inflatable balloon mounted to the catheter shaft and defining a balloon interior volume; and at least five lithotripsy emitter bodies disposed within the balloon interior volume, wherein each emitter body includes: an electrically conductive body mounted about an inner shaft of the catheter; at least one conductor channel configured to route a conductor longitudinally through the emitter body; and an exposed metal region exposing an emitter body surface and a conductor surface through a dielectric encapsulation.

Embodiment 12. The intravascular lithotripsy balloon catheter of embodiment 11, wherein the conductor surface and the emitter body surface have equal or substantially equal surface areas within the exposed metal region, such that repeated electrical discharges produce progressive erosion of the exposed conductor surface along the exposed metal region while maintaining a constant or substantially constant spark gap.

Embodiment 13. The intravascular lithotripsy balloon catheter of embodiment 11 or 12, wherein the at least five lithotripsy emitter bodies includes six emitter bodies arranged longitudinally within the balloon interior volume.

Embodiment 14. The intravascular lithotripsy balloon catheter of embodiment 13, wherein the six emitter bodies are arranged as three emitter pairs, each emitter pair including a proximal emitter body and a distal emitter body.

Embodiment 15. The intravascular lithotripsy balloon catheter of embodiment 14, wherein emitter bodies within each emitter pair are electrically coupled in series such that electrical discharges occur across spark gaps of both emitter bodies during application of a single voltage pulse.

Embodiment 16. The intravascular lithotripsy balloon catheter of embodiment 14, further including at least one bridge conductor extending between emitter bodies of an emitter pair to electrically couple the emitter bodies.

Embodiment 17. The intravascular lithotripsy balloon catheter of embodiment 16, wherein the bridge conductor includes a floating conductor that is not directly connected to a voltage source when no electrical discharge is occurring.

Embodiment 18. The intravascular lithotripsy balloon catheter of embodiment 16 or 17, wherein the bridge conductor is routed through conductor channels of adjacent emitter bodies.

Embodiment 19. The intravascular lithotripsy balloon catheter of any one of embodiments 14-18, further including multiple supply conductors and return conductors extending proximally from the balloon, each emitter pair being associated with a respective supply conductor and a respective return conductor.

Embodiment 20. The intravascular lithotripsy balloon catheter of embodiment 19, wherein each emitter pair is electrically isolated from other emitter pairs along the catheter.

Embodiment 21. The intravascular lithotripsy balloon catheter of any one of embodiments 11-20, wherein each emitter body is configured to route multiple conductors through the at least one conductor channel.

Embodiment 22. The intravascular lithotripsy balloon catheter of any one of embodiment 11-21, wherein the conductor channel of each emitter body is configured to route (i) at least one bridge conductor and (ii) at least one supply or return conductor therethrough while maintaining a common radial envelope between the conductor and the emitter body.

Embodiment 23. The intravascular lithotripsy balloon catheter of any one of embodiments 11-22, wherein the guidewire is a 0.025-inch (0.635 mm) to 0.038-inch (0.965 mm) guidewire.

Embodiment 24. The intravascular lithotripsy balloon catheter of any one of embodiments 11-23, wherein the guidewire is a 0.035-inch (0.889 mm) guidewire.

Embodiment 25. An apparatus configured to deliver lithotripsy intravascularly, the apparatus including: a catheter extending along a longitudinal axis, defining a guidewire lumen configured to receive a 0.035-inch (0.889 mm) guidewire; an inner shaft disposed within the catheter; and at least four emitter modules disposed along the inner shaft, each emitter module including: an emitter body having an emitter body surface extending along the longitudinal axis; at least one discharge conductor having a conductor surface spaced from the emitter body surface by a spark gap; and a dielectric material insulating the at least one discharge conductor from the emitter body surface except at a discharge location including a first exposed discharge surface of the emitter body surface and a second exposed discharge surface of the conductor surface; wherein the emitter body surface is disposed at a first radius from the longitudinal axis and the conductor surface is disposed at a second radius from the longitudinal axis, and wherein the first radius is equal or substantially equal to the second radius such that a spark gap distance between the emitter body surface and the conductor surface remains constant or substantially constant as the conductor surface erodes during repeated spark generation events.

Embodiment 26. The apparatus of embodiment 25, wherein the discharge location includes a window formed in the emitter body that exposes the emitter body surface and the conductor surface across the spark gap.

Embodiment 27. The apparatus of embodiment 25 or 26, wherein the at least one discharge conductor is routed through a channel formed in the emitter body and is partially captured by the emitter body such that the at least one discharge conductor and the emitter body are inscribed within a common radial envelope.

Embodiment 28. The apparatus of any one of embodiments 25-27, wherein each emitter module includes multiple spark gaps.

Embodiment 29. The apparatus of embodiment 28, wherein the multiple spark gaps include two spark gaps positioned circumferentially about the emitter body.

Embodiment 30. The apparatus of any one of embodiments 25-29, wherein six emitter modules are arranged as three emitter pairs disposed within a balloon having a length of approximately or equal to 60 mm.

Embodiment 31. The apparatus of embodiment 30, wherein each emitter pair is electrically isolated from other emitter pairs and is associated with a dedicated supply conductor and a dedicated return conductor.

Embodiment 32. The apparatus of embodiment 30 or 31, wherein the guidewire lumen, the inner shaft, and the six emitter modules are dimensioned such that the catheter is introducible through a 7 French introducer sheath.

Embodiment 33. An apparatus configured to deliver lithotripsy intravascularly, the apparatus including: a catheter shaft extending between a proximal end and a distal end and defining a guidewire lumen sized to receive a 0.035-inch (0.889 mm) guidewire; an inflatable balloon mounted to the catheter shaft and defining a balloon interior volume; and multiple lithotripsy emitter bodies disposed within the balloon interior volume, wherein each emitter body includes: an electrically conductive body mounted about an inner shaft of the catheter; at least one conductor channel configured to route at least a discharge conductor longitudinally through the emitter body; and a charge concentration region exposing, through a dielectric encapsulation, a first exposed discharge surface including an emitter body surface and a second exposed discharge surface including a conductor surface; wherein the first exposed discharge surface and the second exposed discharge surface have equal or substantially equal surface areas within the charge concentration region, such that repeated electrical discharges produce progressive erosion of the conductor surface along the charge concentration region while maintaining a constant or substantially constant spark gap.

Embodiment 34. The apparatus of embodiment 33, wherein the multiple lithotripsy emitter bodies includes six emitter bodies arranged longitudinally within the balloon interior volume.

Embodiment 35. The apparatus of embodiment 34, wherein the six emitter bodies are arranged as three emitter pairs, each emitter pair including a proximal emitter body and a distal emitter body.

Embodiment 36. The apparatus of embodiment 35, wherein emitter bodies within each emitter pair are electrically coupled in series such that electrical discharges occur across spark gaps of both emitter bodies during application of a single voltage pulse.

Embodiment 37. The apparatus of embodiment 35, further including at least one bridge conductor extending between emitter bodies of an emitter pair to electrically couple the emitter bodies.

Embodiment 38. The apparatus of embodiment 37, wherein the at least one bridge conductor includes a floating conductor that is not directly connected to a voltage source when no electrical discharge is occurring.

Embodiment 39. The apparatus of embodiment 37 or 38, wherein at least one of the at least one bridge conductor is routed through conductor channels of adjacent emitter bodies.

Embodiment 40. The apparatus of any one of embodiments 35-39, further including multiple supply conductors and at least one return conductor extending proximally from the inflatable balloon, each emitter pair being associated with a respective supply conductor and a respective return conductor.

Embodiment 41. The apparatus of embodiment 40, wherein each emitter pair is electrically isolated from other emitter pairs along the catheter.

Embodiment 42. The apparatus of any one of embodiments 34-41, wherein each emitter body is configured to route multiple conductors through the at least one conductor channel.

Embodiment 43. The apparatus of any one of embodiments 34-42, wherein the at least one conductor channel of each emitter body is configured to route at least one bridge conductor, and at least one supply or return conductor therethrough while maintaining a common radial envelope between the conductor and the emitter body.

Embodiment 44. The apparatus of any one of embodiments 25-43, wherein the dielectric material both encapsulates the emitter body and at least one conductor except at the exposed discharge surfaces, and also electrically isolates the emitter body from the at least one conductor.

Embodiment 45. An apparatus configured to deliver lithotripsy intravascularly, the apparatus including: a catheter body extending along a longitudinal centerline; an emitter module disposed on the catheter body, the emitter module including: an emitter body including a discharge aperture, the discharge aperture including a first exposed discharge surface; and a discharge conductor including a second exposed discharge surface within the discharge aperture; wherein an arcuate surface concentric with the longitudinal centerline passes through both the second exposed discharge surface and the first exposed discharge surface.

Embodiment 46. An apparatus configured to deliver lithotripsy intravascularly, the apparatus including: a catheter body extending along a longitudinal centerline; an emitter module disposed on the catheter body, the emitter module including: an emitter body extending along a longitudinal axis, the emitter body including a conductor channel extending longitudinally and a discharge aperture opening to the conductor channel, at least a portion of the discharge aperture including a first exposed discharge surface; a discharge conductor extending through the conductor channel, the discharge conductor including a second exposed discharge surface within the discharge aperture; and an encapsulating dielectric material separating the emitter body from the discharge conductor such that electrical discharge between the emitter body and the discharge conductor is physically inhibited except between the first exposed discharge surface and the second exposed discharge surface.

Embodiment 47. An apparatus configured to deliver lithotripsy intravascularly, the apparatus including: a catheter body extending along a longitudinal centerline; an emitter module disposed on the catheter body, the emitter module including: an emitter body including a discharge aperture, the discharge aperture including a first exposed discharge surface; and a discharge conductor including a second exposed discharge surface within the discharge aperture, wherein a ratio of a surface area of the first exposed discharge surface to a surface area of the second exposed discharge surface is less than or equal to 10:1.

Embodiment 48. An apparatus configured to deliver lithotripsy intravascularly, the apparatus including: a catheter body defining a longitudinal centerline; and an emitter module including a first electrode member and a second electrode member disposed on the catheter body, wherein: the first electrode member and the second electrode member include opposing exposed discharge surfaces positioned to define an electrical discharge region; insulating material surrounds the first electrode member and the second electrode member and bounds the exposed discharge surfaces such that electrical discharge is inhibited outside the electrical discharge region; and the exposed discharge surfaces are positioned such that they define: a first edge-to-edge distance between the exposed discharge surfaces that is less than a second edge-to-edge distance between the exposed discharge surfaces at a laterally offset location, the first edge-to-edge distance determining a breakdown voltage initiating electrical discharge, a channel width defined by opposing channel walls of the first electrode member bounding a discharge aperture, the channel width being narrower than a circumferential extent of the first electrode member outside the discharge aperture, such that plasma expansion during electrical discharge is laterally constrained by the channel walls; and the breakdown voltage initiating electrical discharge is determined by the first edge-to-edge distance independently of the channel width, and a magnitude of discharge current following breakdown is determined by the channel width independently of the first edge-to-edge distance.

Embodiment 49. The apparatus of embodiment 48, wherein the first electrode member includes an emitter body, and the second electrode member includes a conductor.

Embodiment 50. An apparatus configured to deliver lithotripsy intravascularly, the apparatus including: an emitter module including: an emitter body including a body with an annular (e.g., interrupted annulus) cross sectional shape extending along a longitudinal axis and configured to be disposed on a catheter body, the emitter body including an outer surface and an inner surface; a conductor channel extending longitudinally through the emitter body; a discharge aperture opening to the conductor channel, the discharge aperture including a first discharge surface, the first discharge surface including longitudinally extending symmetric surfaces on either side of a plane passing through the longitudinal axis of the emitter body and through a center of the discharge aperture; and a discharge conductor extending through the conductor channel, the discharge conductor including a second discharge surface within the discharge aperture, the discharge conductor having an outermost diameter at least eighty percent of a thickness of the emitter body, wherein: the discharge conductor, the first discharge surface, and the second discharge surface are all positioned within a volumetric shell bounded by the outer surface of the emitter body and the inner surface of the emitter body, in a retention region, a width of the conductor channel at the outer surface is less than a width of the conductor channel halfway between the outer surface and the inner surface, and at least a portion of the first discharge surface and the second discharge surface are exposed.

Embodiment 51. An apparatus configured to deliver lithotripsy intravascularly, the apparatus including: a catheter body extending along a longitudinal centerline; an emitter module disposed on the catheter body, the emitter module including: an emitter body including a conductor channel; and a discharge conductor disposed within the conductor channel; wherein the conductor channel follows a non-linear path in a circumferential direction relative to the emitter body such that the discharge conductor deviates from a straight radial or chordal alignment.

Embodiment 52. The apparatus of embodiment 51, wherein edge-to-edge distances between the discharge conductor and opposing surfaces of the emitter body vary along the conductor channel.

Embodiment 53. The apparatus of any one of embodiments 51-52, wherein the emitter body defines a first exposed discharge surface within the conductor channel, and the discharge conductor defines a second exposed discharge surface within the conductor channel, such that the first exposed discharge surface and the second exposed discharge surface are positioned adjacent to one another within the conductor channel.

Embodiment 54. The apparatus of any one of embodiments 25-53, wherein a ratio of surface areas of the exposed surfaces is less than or equal to 5:1.

Embodiment 55. The apparatus of embodiment 54, wherein the ratio is less than or equal to 3:1.

Embodiment 56. The apparatus of embodiment 55, wherein the ratio is less than or equal to 2:1.

Embodiment 57. The apparatus of embodiment 56, wherein a surface area of the first exposed discharge surface is equal or substantially equal to a surface area of the second exposed discharge surface.

Embodiment 58. The apparatus of any one of embodiments 54-57, wherein the ratio of the surface area of the first exposed discharge surface to the surface area of the second exposed discharge surface is less than or equal to 3:1 prior to a first discharge event.

Embodiment 59. The apparatus of any one of embodiments 54-57, wherein the ratio of the surface area of the first exposed discharge surface to the surface area of the second exposed discharge surface is less than or equal to 10:1 after a final discharge event of multiple discharge events.

Embodiment 60. The apparatus of any one of embodiments 25-59, wherein at least one of the exposed discharge surfaces includes multiple exposed discharge surfaces positioned on opposing sides of a discharge conductor within a discharge aperture.

Embodiment 61. The apparatus of any one of embodiments 54-60, wherein the ratio of the surface areas varies over multiple discharge events as electrode material erodes during repeated spark formation cycles.

Embodiment 62. The apparatus of any one of embodiments 25-61, wherein at least one of the exposed discharge surfaces is defined by a cavity in a dielectric material otherwise encapsulating the emitter module and electrically isolating from each other conductive surfaces of the emitter module.

Embodiment 63. The apparatus of any one of embodiments 25-62, wherein the exposed discharge surfaces are contained within a spark initiation volumetric shell concentric with the longitudinal centerline, the spark initiation volumetric shell having an inner radius defined by a lowermost first exposed discharge surface and an outer radius defined by an outermost first exposed discharge surface.

Embodiment 64. The apparatus of any one of embodiments 25-63, wherein the emitter module includes a body with an interrupted annular-shaped cross section extending at least partially circumferentially about the longitudinal centerline.

Embodiment 65. The apparatus of embodiment 64, wherein the first exposed discharge surface and the second exposed discharge surface are each positioned at a common spark initiation radius relative to the longitudinal centerline, such that an at least partially arcuate surface at the common spark initiation radius passes through both the first exposed discharge surface and the second exposed discharge surface.

Embodiment 66. The apparatus of embodiment 65, wherein the inner radius of the spark initiation volumetric shell is greater than a distance from the longitudinal centerline to an inner surface of the emitter body.

Embodiment 67. The apparatus of any one of embodiments 25-66, wherein a first edge-to-edge distance between a pair of the exposed discharge surfaces is less than a median electrode distance between the pair.

Embodiment 68. The apparatus of any one of embodiments 25-67, wherein a first edge-to-edge distance between a pair of the exposed discharge surfaces decreases along the longitudinal centerline progressing away from a terminal end of the discharge conductor.

Embodiment 69. The apparatus of any one of embodiments 67-68, wherein the first edge-to-edge distance is defined by a diameter of a discharge conductor, an inner diameter of an emitter body, and a radial depth of insulation termination.

Embodiment 70. The apparatus of any one of embodiments 67-69, wherein a ratio of the median electrode distance to the first edge-to-edge distance is greater than 1:1.

Embodiment 71. The apparatus of any one of embodiments 25-70, wherein a first edge-to-edge distance between a pair of the exposed discharge surfaces is constant or substantially constant along an erosion path of a discharge conductor during repeated spark formation cycles.

Embodiment 72. The apparatus of any one of embodiments 25-71, wherein a first edge-to-edge distance between a pair of the exposed discharge surfaces at a first spark initiation site is less than a second edge-to-edge distance between the exposed discharge surfaces at a laterally offset spark initiation site, such that electrical discharge initiates preferentially at the first spark initiation site prior to initiating at the laterally offset spark initiation site.

Embodiment 73. The apparatus of any one of embodiments 48-49, wherein the channel width is less than one-half of a circumferential extent of the first electrode member.

Embodiment 74. The apparatus of any one of embodiments 48-49 and 73, wherein reduction of the channel width increases discharge current confinement without reducing the first edge-to-edge distance.

Embodiment 75. The apparatus of any one of embodiments 48-49 and 73-74, wherein reduction of the first edge-to-edge distance reduces the breakdown voltage without increasing the channel width.

Embodiment 76. The apparatus of any one of embodiments 48-49 and 73-75, wherein the channel width is independent of a radial depth of insulation removal that defines a first edge-to-edge distance between a pair of exposed discharge surfaces.

Embodiment 77. The apparatus of embodiment 76, wherein the first edge-to-edge distance is defined by: (i) a diameter of the second electrode member, (ii) an inner diameter of the first electrode member, (iii) a radial depth of insulation termination, and (iv) a width of a channel formed in the first electrode member.

Embodiment 78. The apparatus of any one of embodiments 48-49, and 73-77, wherein the channel width constrains a lateral dimension of plasma expansion during discharge independently of a spacing between the first electrode member and the second electrode member.

Embodiment 79. The apparatus of any one of embodiments 48-49, and 73-78, wherein the first electrode member includes a ring electrode member and the second electrode member includes a wire electrode member disposed within a conductor channel of the ring electrode member.

Embodiment 80. The apparatus of any one of embodiments 25-47, and 49-79, wherein the discharge conductor, the first exposed discharge surface, and the second exposed discharge surface are all positioned within a volumetric shell bounded by an outer surface of the emitter body and an inner surface of the emitter body.

Embodiment 81. The apparatus of any one of embodiments 46 and 49-80, wherein the conductor channel includes a shaped internal surface such that a width of the conductor channel varies at different radial locations relative to the longitudinal centerline.

Embodiment 82. The apparatus of embodiment 81, wherein the shaped internal surface includes a curved internal surface.

Embodiment 83. The apparatus of any one of embodiments 81-82, wherein the shaped internal surface includes a tapered internal surface.

Embodiment 84. The apparatus of any one of embodiments 25-47, and 49-83, wherein an outermost diameter of the discharge conductor is equal to a thickness of the emitter body measured between an outer surface and an inner surface of the emitter body.

Embodiment 85. The apparatus of any one of embodiments 25-47, and 49-84, wherein the discharge aperture includes longitudinally extending symmetric surfaces on either side of a plane passing through the longitudinal centerline and through a center of the discharge aperture.

Embodiment 86. The apparatus of any one of embodiments 81-85, wherein the shaped internal surface retains the discharge conductor within the conductor channel without requiring an external support member.

Embodiment 87. The apparatus of any one of embodiments 51-86, wherein the non-linear path includes a continuous curvature in the circumferential direction.

Embodiment 88. The apparatus of any one of embodiments 51-87, wherein the non-linear path includes one or more inflection regions along the conductor channel.

Embodiment 89. The apparatus of any one of embodiments 33-44, 46, and 49-88, wherein the conductor channel follows a non-linear path in at least two dimensions.

Embodiment 90. The apparatus of any one of embodiments 33-44, 46, and 49-89, wherein the conductor channel varies in cross-sectional width along the non-linear path.

Embodiment 91. The apparatus of any one of embodiments 51-90, wherein the non-linear path defines a varying edge-to-edge distance between the discharge conductor and the opposing surfaces of the emitter body at multiple locations along the conductor channel.

Embodiment 92. The apparatus of any one of embodiments 88-91, wherein spark initiation migrates along the non-linear path during progressive electrode erosion, distributing erosion along the conductor channel.

Embodiment 93. The apparatus of any one of embodiments 25-47 and 49-92, wherein the emitter body includes multiple conductor channels, each conductor channel housing a respective discharge conductor.

Embodiment 94. The apparatus of any one of embodiments 25-93, wherein the emitter module is one of multiple emitter modules positioned along the catheter body.

Embodiment 95. The apparatus of embodiment 94, wherein the multiple emitter modules include four emitter modules.

Embodiment 96. The apparatus of any one of embodiments 94-95, wherein the multiple emitter modules include six emitter modules.

Embodiment 97. The apparatus of any one of embodiments 94-96, wherein the multiple emitter modules are disposed along a longitudinally extending shaft of the catheter body.

Embodiment 98. The apparatus of any one of embodiments 25-47, and 49-97, wherein the emitter body includes multiple discharge apertures and at least a corresponding number of discharge conductors.

Embodiment 99. The apparatus of any one of embodiments 94-98, further including at least one floating bridge wire configured to electrically connect, during occurrence of an electrical arc across a spark gap of the emitter module, the emitter module to a distal emitter module of the multiple emitter modules.

Embodiment 100. The apparatus of embodiment 99, wherein the at least one floating bridge wire is shorter in length by a factor of at least 100 than one or more conductors electrically connected to an electrical source prior to the electrical arc.

Embodiment 101. The apparatus of any one of embodiments 25-100, further including a pulse generator connected to the emitter module, the pulse generator configured to vary an applied voltage.

Embodiment 102. The apparatus of embodiment 101, further including a control module configured to adjust a voltage magnitude as a function of a number of emission cycles.

Embodiment 103. The apparatus of any one of embodiments 101-102, wherein the pulse generator is configured to incrementally increase applied voltage per N discharge events according to a predetermined voltage progression schedule corresponding to expected geometric changes along a spark progression path of the emitter module. In a further embodiment, the voltage progression schedule accounts for one or more of: progressive electrode erosion along the spark progression path, gas accumulation within a balloon interior volume from prior discharge events, and/or expected variation in discharge current magnitude along the spark progression path.

Embodiment 104. The apparatus of any one of embodiments 101-103, wherein the pulse generator is configured to operate within a voltage range of 2 kV to 5 kV throughout a lifetime of the emitter module.

Embodiment 105. The apparatus of any one of embodiments 94-104, wherein a transfer function of a first emitter module of the multiple emitter modules defines a steeper voltage rise over time than a transfer function of a second emitter module of the multiple emitter modules.

Embodiment 106. The apparatus of any one of embodiments 25-105, wherein the catheter body defines a guidewire lumen extending along the longitudinal centerline.

Embodiment 107. The apparatus of any one of embodiments 25-106, further including a balloon disposed about the catheter body and enclosing the emitter module.

Embodiment 108. The apparatus of any one of embodiments 25-107, wherein the apparatus is configured with a crossing distance less than or equal to 0.085 inches (2.159 mm).

Embodiment 109. The apparatus of any one of embodiments 25-108, wherein the apparatus is configured with a crossing distance less than or equal to 0.080 inches (2.032 mm).

Embodiment 110. The apparatus of any one of embodiments 25-109, wherein the apparatus is configured with a crossing distance less than or equal to 0.065 inches (1.651 mm).

Embodiment 111. The apparatus of any one of embodiments 25-110, wherein the apparatus is configured with a crossing distance less than or equal to 0.061 inches (1.549 mm).

Embodiment 112. The apparatus of any one of embodiments 25-111, wherein the apparatus is configured with a crossing distance less than or equal to 0.060 inches (1.524 mm).

Embodiment 113. The apparatus of any one of embodiments 25-112, wherein the apparatus is configured with a crossing distance less than or equal to 0.050 inches (1.27 mm).

Embodiment 114. The apparatus of any one of embodiments 25-47 and 49-113, wherein an encapsulating dielectric material includes a conformal coating applied over the emitter body and the discharge conductor.

Embodiment 115. The apparatus of embodiment 114, wherein the encapsulating dielectric material is disposed between the emitter body and the discharge conductor along a length of the conductor channel outside the discharge aperture.

Embodiment 116. The apparatus of embodiment 115, wherein the encapsulating dielectric material includes an epoxy with at least 2% post-cure elongation at break.

Embodiment 117. The apparatus of any one of embodiments 25-116, wherein the catheter body includes a shaft including polyimide.

Embodiment 118. The apparatus of any one of embodiments 25-117, wherein the emitter body is bonded to a shaft of the catheter body by an adhesive including an ultraviolet adhesive with shadow curing properties.

Embodiment 119. The apparatus of any one of embodiments 25-118, wherein conductors connecting the emitter module to an electrical source are disposed along an outer surface of a shaft of the catheter body.

Embodiment 120. The apparatus of any one of embodiments 25-47, and 48-119, wherein the discharge conductor is positioned sub-flush relative to an outer surface of the emitter body such that no portion of the discharge conductor protrudes beyond the outer surface of the emitter body.

Embodiment 121. The apparatus of any one of embodiments 25-120, wherein each of at least two of the exposed discharge surfaces are defined by a region of the discharge aperture from which an encapsulating dielectric material is absent.

Embodiment 122. The apparatus of embodiment 121, wherein the region of the discharge aperture from which the encapsulating dielectric material is absent is formed by removal of a portion of the encapsulating dielectric material after application of the encapsulating dielectric material over the emitter body and the discharge conductor.

Embodiment 123. The apparatus of any one of embodiments 46, and 48-122, wherein the conductor channel defines a spark progression path along which spark initiation locations migrate during progressive electrode erosion.

Embodiment 124. The apparatus of any one of embodiments 25-47, and 49-123, wherein an encapsulating dielectric material bounds the first exposed discharge surface and the second exposed discharge surface, wherein electrical discharge along insulated portions of the emitter body is inhibited such that the discharge conductor guides erosion along a predetermined spark progression path.

Embodiment 125. The apparatus of any one of embodiments 123-124, wherein a first edge-to-edge distance between the second exposed discharge surface and the first exposed discharge surface varies over multiple discharge events as electrode material erodes along the spark progression path.

Embodiment 126. The apparatus of any one of embodiments 123-125, wherein a breakdown voltage initiating electrical discharge changes monotonically as spark initiation locations migrate along the spark progression path.

Embodiment 127. The apparatus of any one of embodiments 123-126, wherein the spark progression path follows a non-linear path of the conductor channel with spatial variation in edge-to-edge distances along the spark progression path.

Embodiment 128. The apparatus of any one of embodiments 126-127, further including a voltage controller configured such that an applied voltage is adjusted over multiple spark events according to a predetermined progression schedule corresponding to an expected spark gap size at a present position along the spark progression path.

Embodiment 129. The apparatus of any one of embodiments 25-128, further including a degassing membrane disposed about the catheter body.

Embodiment 130. The apparatus of embodiment 129, wherein the degassing membrane includes a semi-permeable polymer membrane.

Embodiment 131. The apparatus of any one of embodiments 25-130, wherein multiple emitter modules are electrically connected in series such that a single applied voltage pulse causes sequential spark generation across multiple spark gaps of the multiple emitter modules.

Embodiment 132. The apparatus of embodiment 131, wherein adjacent emitter modules of the multiple emitter modules are electrically connected by bridge conductors, each bridge conductor configured to conduct current from a first emitter module to a second emitter module upon occurrence of an electrical arc across a spark gap of the first emitter module.

Embodiment 133. The apparatus of any one of embodiments 106-132, wherein the guidewire lumen is configured as an over-the-wire guidewire lumen.

Embodiment 134. The apparatus of any one of embodiments 106-133, wherein the guidewire lumen is configured as a rapid exchange guidewire lumen.

Embodiment 135. The apparatus of any one of embodiments 25-47, and 49-134, wherein a width of the discharge aperture is between 1.0 and 3.0 times a diameter of the discharge conductor.

Embodiment 136. The apparatus of any one of embodiments 25-47, and 49-135, wherein a depth of the discharge aperture is between 0.3 and 0.7 times a wall thickness of the emitter body.

Embodiment 137. The apparatus of any one of embodiments 46-136, wherein a shaped internal surface of the conductor channel mechanically retains the discharge conductor without adhesive bonding.

Embodiment 138. The apparatus of any one of embodiments 25-137, wherein: a conductor channel extends both radially inward from an outermost radial surface of the emitter body and longitudinally inward from at least one end of the emitter body such that the conductor channel defines both a path of at least one of the conductors and a predetermined spark progression path, and a circumferential width of the conductor channel is narrower than a diameter of the at least one of the conductors in a conductor retention region of the conductor channel, distal to an initial spark gap location, such that multiple successive spark gaps as the at least one of the conductors erodes along the predetermined spark progression path have a first edge-to-edge distance greater than when the discharge conductor erodes into the conductor retention region.

Embodiment 139. A method of initiating electrical discharge in a catheter-based energy delivery device, the method including: providing an emitter module according to any one of embodiments 25-138, at least a pair of the exposed discharge surfaces positioned as opposing discharge surfaces and bounded by insulating material, the opposing exposed discharge surfaces defining multiple spark initiation sites; positioning the opposing exposed discharge surfaces such that a first edge-to-edge distance between a first pair of opposing spark initiation sites is less than a second edge-to-edge distance between a second pair of opposing spark initiation sites at a laterally offset location relative to the first pair; and providing connections for applying an electrical potential to the emitter module across the opposing exposed discharge surfaces, such that electrical discharge initiates preferentially at the first pair of opposing spark initiation sites prior to initiating at the second pair of opposing spark initiation sites.

Embodiment 140. A method of delivering lithotripsy intravascularly, the method including: providing an apparatus according to any of embodiments 25-138, the apparatus including the catheter body and multiple of the emitter modules positioned along the catheter body, each emitter module including an encapsulating dielectric material insulating conductive surfaces corresponding to the first exposed discharge surface and the second exposed discharge surface from each other except at the first exposed discharge surface and the second exposed discharge surface, cooperating to form a spark gap; deploying the catheter body within a blood vessel such that the multiple emitter modules are positioned adjacent to a target treatment site; connecting the multiple emitter modules to a pulse generator; and operating the pulse generator such that the multiple emitter modules generate sparks resulting in delivery of pressure waves intravascularly at the target treatment site.

Embodiment 141. The method of embodiment 140, the method including: providing the apparatus of any one of embodiments 25-138 such that at least one of the multiple emitter modules includes an emitter body, discharge conductor, and dielectric material cooperate to define a predetermined spark progression path along which spark initiation locations migrate during progressive electrode erosion; applying a first voltage during a first set of discharge events, the first voltage corresponding to a first spark gap distance at a first spark initiation location along the predetermined spark progression path; and applying a second voltage during a second set of discharge events subsequent to the first set of discharge events, the second voltage being different than the first voltage and corresponding to a second spark gap distance at a second spark initiation location along the predetermined spark progression path, the second spark gap distance being different than the first spark gap distance, wherein a difference in applied voltage varies directly with the difference in spark gap distance.

Embodiment 142. The method of embodiment 141, wherein the first voltage and the second voltage are selected such that a magnitude of pressure waves generated during the first set of discharge events and the second set of discharge events is constant or substantially consistent.

Embodiment 143. The method of embodiment 140-142, wherein the method is used for treatment of peripheral artery disease.

Embodiment 144. The method of any one of embodiments 140-143, wherein the method is used to modify vessel calcification to enhance effectiveness of a subsequent stent deployment in a blood vessel.

Embodiment 145. The method of any one of embodiments 140-144, further including adjusting a voltage magnitude applied by the pulse generator as a function of a number of discharge events generated by the multiple emitter modules.

Embodiment 146. The method of any one of embodiments 140-145, further including setting a power profile prior to operating the pulse generator, the power profile defining at least one of a voltage magnitude, a pulse duration, or a number of pulses.

Embodiment 147. The method of any one of embodiments 140-146, wherein operating the pulse generator includes applying a predetermined number N of voltage pulses to the multiple emitter modules.

Embodiment 148. The method of embodiment 147, further including, after applying the predetermined number N of voltage pulses, adjusting a voltage magnitude and applying an additional set of voltage pulses at the adjusted voltage magnitude.

Embodiment 149. A method of manufacturing an emitter module of an intravascular lithotripsy device, the method including: providing an emitter body including a conductor channel; disposing a discharge conductor within the conductor channel; applying an encapsulating dielectric material over the emitter body and the discharge conductor; and forming a first exposed discharge surface and a second exposed discharge surface. In a further embodiment, the method may include bonding the emitter body to a shaft of a catheter body using an adhesive.

Embodiment 150. The method of embodiment 149, wherein the emitter module is according to any one of embodiments 25-137.

Embodiment 151. The method of any one of embodiments 149-150, wherein forming the first exposed discharge surface and the second exposed discharge surface includes removing a portion of the encapsulating dielectric material.

Embodiment 152. The method of embodiment 151, wherein the removing includes laser ablation, mechanical cutting, and/or chemical etching.

Embodiment 153. The method of any one of embodiments 149-152 wherein forming the first exposed discharge surface and the second exposed discharge surface includes preventing deposition of the encapsulating dielectric material at the discharge aperture during the applying.

Embodiment 154. The method of any one of embodiments 149-153, wherein the encapsulating dielectric material is applied after the emitter body and the discharge conductor are assembled to a shaft of the catheter body.

Embodiment 155. The method of any one of embodiments 149-154, wherein the forming defines a first edge-to-edge distance between the first exposed discharge surface and the second exposed discharge surface that is less than a second edge-to-edge distance between the first exposed discharge surface and the second exposed discharge surface at a laterally offset location within the discharge aperture.

Embodiment 156. The method of any one of embodiments 149-155, wherein the forming defines a ratio of a surface area of the first exposed discharge surface to a surface area of the second exposed discharge surface that is less than or equal to 10:1.

Embodiment 157. The method of any one of embodiments 149-156, wherein the forming defines a predetermined spark progression path along which spark initiation locations migrate during progressive electrode erosion.

Embodiment 158. The method of any one of embodiments 149-157, wherein the conductor channel includes a shaped internal surface such that a width of the conductor channel varies at different radial locations relative to a longitudinal centerline of the catheter body.

Embodiment 159. The method of any one of embodiments 149-158, further including bonding the emitter body to a shaft of the catheter body using an adhesive including a photopolymerized adhesive with shadow curing properties.

Embodiment 160. The method of any one of embodiments 149-159, wherein the encapsulating dielectric material includes photopolymerized adhesive.

Embodiment 161. A method of treating a calcified lesion in a peripheral vessel of a patient, the method including: providing an intravascular lithotripsy assembly including a catheter defining a guidewire lumen sized to receive a guidewire of between 0.025 inch (0.635 mm) and 0.095 inch (2.413 mm) in diameter and an inflatable balloon mounted to the catheter, the inflatable balloon containing emitter modules disposed along an inner shaft of the catheter; advancing the catheter over a guidewire of between 0.025 inch (0.635 mm) and 0.095 inch (2.413 mm) in diameter through a peripheral vessel of the patient until the inflatable balloon is positioned at the calcified lesion; inflating the inflatable balloon with an inflation medium such that an outer surface of the balloon engages a wall of the vessel; and operating a pulse generator coupled to the emitter modules to apply one or more high-voltage pulses, thereby causing electrical discharge across one or more spark gaps of the emitter modules and generating acoustic pressure waves that fracture the calcified lesion.

Embodiment 162. The method of embodiment 161 containing one or more components according to any one of embodiments 1-138 and/or manufactured according to 149-160.

Embodiment 163. The method of embodiment 161 or 162, operated according to any one of embodiments 139-148.

As used in the claims, comprising and consisting are used in the United States open-ended and closed-ended sense, respectively.

It will be understood that various modifications can be made within the scope of this disclosure. Embodiments depict illustrative combinations of disclosed features, components, and/or steps. Any combination of disclosed features is expressly contemplated unless specifically excluded or required by the context. For example, one or more advantageously results may be achieved if components are removed, added, multiplied, scaled, and/or rearranged, and/or if steps in a method are omitted, added, repeated, and/or performed in a different order. Therefore, other implementations are contemplated.

1. An intravascular lithotripsy system, comprising:
a catheter extending along a longitudinal axis, defining a guidewire lumen configured to receive a 0.025-inch (0.635 mm) to 0.095-inch (2.413 mm) guidewire;
an inner shaft disposed within the catheter; and
at least four emitter modules disposed along the inner shaft, each emitter module comprising:
an emitter body having an emitter body surface extending along the longitudinal axis;
at least one conductor having a conductor surface spaced from the emitter body surface by a spark gap; and
a dielectric material insulating the at least one conductor from the emitter body surface except at a discharge location.

2. The intravascular lithotripsy system of claim 1, wherein the emitter body surface is disposed at a first radius from the longitudinal axis and the conductor surface is disposed at a second radius from the longitudinal axis and wherein the first radius is equal or substantially equal to the second radius such that a spark gap distance between the emitter body surface and the conductor surface remains constant or substantially constant as the conductor surface erodes during repeated spark generation events.

3. The intravascular lithotripsy system of claim 1, wherein the discharge location comprises a window formed in the emitter body that exposes the emitter body surface and the conductor surface across the spark gap.

4. The intravascular lithotripsy system of claim 1, wherein the at least one conductor is routed through a channel formed in the emitter body and is partially captured by the emitter body such that the conductor and the emitter body are inscribed within a common radial envelope.

5. The intravascular lithotripsy system of claim 1, wherein each emitter module comprises two spark gaps positioned circumferentially about the emitter body.

6. The intravascular lithotripsy system of claim 1, wherein six emitter modules are arranged as three emitter pairs disposed within a balloon having a length of 60 mm or approximately 60 mm.

7. The intravascular lithotripsy system of claim 6, wherein each emitter pair is electrically isolated from other emitter pairs and is associated with a dedicated supply conductor and a dedicated return conductor.

8. The intravascular lithotripsy system of claim 6, wherein the guidewire lumen, the inner shaft, and the six emitter modules are dimensioned such that the catheter is introducible through a 7 French introducer sheath.

9. The intravascular lithotripsy system of claim 1, wherein the guidewire is a 0.025-inch (0.635 mm) to 0.038-inch (0.965 mm) guidewire.

10. The intravascular lithotripsy system of claim 1, wherein the guidewire is a 0.035-inch (0.889 mm) guidewire.

11. An intravascular lithotripsy balloon catheter comprising:
a catheter shaft extending between a proximal end and a distal end and defining a guidewire lumen sized to receive a 0.025-inch (0.635 mm) to 0.095-inch (2.413 mm) guidewire;
an inflatable balloon mounted to the catheter shaft and defining a balloon interior volume; and
at least five lithotripsy emitter bodies disposed within the balloon interior volume,
wherein each emitter body comprises:
an electrically conductive body mounted about an inner shaft of the catheter;
at least one conductor channel configured to route a conductor longitudinally through the emitter body; and
an exposed metal region exposing an emitter body surface and a conductor surface through a dielectric encapsulation.

12. The intravascular lithotripsy balloon catheter of claim 11, wherein the conductor surface and the emitter body surface have equal or substantially equal surface areas within the exposed metal region, such that repeated electrical discharges produce progressive erosion of the exposed conductor surface along the exposed metal region while maintaining a constant or substantially constant spark gap.

13. The intravascular lithotripsy balloon catheter of claim 11, wherein the at least five lithotripsy emitter bodies comprises six emitter bodies arranged longitudinally within the balloon interior volume.

14. The intravascular lithotripsy balloon catheter of claim 13, wherein the six emitter bodies are arranged as three emitter pairs, each emitter pair comprising a proximal emitter body and a distal emitter body.

15. The intravascular lithotripsy balloon catheter of claim 14, wherein emitter bodies within each emitter pair are electrically coupled in series such that electrical discharges occur across spark gaps of both emitter bodies during application of a single voltage pulse.

16. The intravascular lithotripsy balloon catheter of claim 14, further comprising at least one bridge conductor extending between emitter bodies of an emitter pair to electrically couple the emitter bodies.

17. The intravascular lithotripsy balloon catheter of claim 16, wherein the bridge conductor comprises a floating conductor that is not directly connected to a voltage source when no electrical discharge is occurring.

18. The intravascular lithotripsy balloon catheter of claim 16, wherein the bridge conductor is routed through conductor channels of adjacent emitter bodies.

19. The intravascular lithotripsy balloon catheter of claim 14, further comprising a plurality of supply conductors and return conductors extending proximally from the balloon, each emitter pair being associated with a respective supply conductor and a respective return conductor.

20. The intravascular lithotripsy balloon catheter of claim 19, wherein each emitter pair is electrically isolated from other emitter pairs along the catheter.

21. The intravascular lithotripsy balloon catheter of claim 11, wherein each emitter body is configured to route multiple conductors through the at least one conductor channel.

22. The intravascular lithotripsy balloon catheter of claim 11, wherein the conductor channel of each emitter body is configured to route (i) at least one bridge conductor and (ii) at least one supply or return conductor therethrough while maintaining a common radial envelope between the conductor and the emitter body.

23. The intravascular lithotripsy balloon catheter of claim 11, wherein the guidewire is a 0.025-inch to 0.038-inch (0.635 mm to 0.965 mm) guidewire.

24. The intravascular lithotripsy balloon catheter of claim 11, wherein the guidewire is a 0.035-inch (0.889 mm) guidewire.
